(19) United States
(12) Patent Application Publication
(10) Pub. No.: US 2022/0363680 A1
(43) Pub. Date: Nov. 17, 2022

(54) PROCESSES FOR THE SYNTHESIS OF VALBENAZINE

(71) Applicant: Neurocrine Biosciences, Inc., San Diego (US)

(72) Inventors: John TUCKER, San Diego, CA (US); David KUCERA, San Diego, CA (US); Donald HETTINGER, San Diego, CA (US); Brian M. COCHRAN, San Diego, CA (US); Shawn BRANUM, San Diego, CA (US); Jackie LE, San Diego, CA (US); Kevin MCGEE, San Diego, CA (US)

(21) Appl. No.: 17/641,705

(22) PCT Filed: Sep. 11, 2020
(86) PCT No.: PCT/US2020/050537

§ 371 Date: Mar. 9, 2022

Related U.S. Application Data
(60) Provisional application No. 62/900,153, filed on Sep. 13, 2019.

(51) Int. Cl. C07D 471/04; A61K 47/12; A61K 47/36

(52) U.S. Cl. CPC C07D 471/04; A61K 47/12; A61K 47/36

(57) ABSTRACT

The present application relates to processes for making (2R,3R,11bR)-3-isobutyl-9,10-dimethoxy-2,3,4,6,7,11b -hexahydro-1H-pyrido[2,1-a]isoquinolin-2-yl (S)-2-amino-3-methylbutanoate di(4-methylbenzenesulfonate), which is an inhibitor of vesicular monoamine transporter 2 (VMAT2) useful in the treatment of hyperkinetic movement disorders such as tardive dyskinesia (TD).

### BACKGROUND

### Technical Field

The present application relates to processes for making (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate), which is an inhibitor of vesicular monoamine transporter 2 (VMAT2) useful in the treatment of hyperkinetic movement disorders such as tardive dyskinesia (TD).

### Description of the Related Technology

Ingrezza®, the first FDA approved therapy for patients suffering from Tardive Dyskinesia, a hyperkinetic movement disorder, contains Valbenazine, present as Valbenazine ditosylate. A potent and selective VMAT2 inhibitor, Valbenazine[(S)-2-amino-3-methyl-butyric acid (2R,3R,1I1bR)-3-isobutyl-9,10-dimethoxy-1,3,4,6,7,11b-hexahydro-2H-pyrido[2,1-a]isoquinolin-2-yl ester] is the purified prodrug of the (+)-α-isomer of dihydrotetrabenazine. The structure of Valbenazine ditosylate is depicted herein as Formula I.

Valbenazine, as well as its preparation and use, has been described in U.S. Pat. Nos. 8,039,627; 8,357,697; and 10,160,757, each of which is incorporated herein by reference in its entirety. Certain salts and crystal forms for Valbenazine have been described in WO2017/075340, and certain formulations for Valbenazine have been described in WO2019/060322, each of which is incorporated herein by reference in its entirety.

Due to the high demand for and usefulness of Ingrezza, there is a need for development of new processes for its manufacture, particularly more environmentally friendly processes. This application is directed towards this need and others.

### SUMMARY

The present application provides, inter alia, processes for preparing a compound of Formula I.

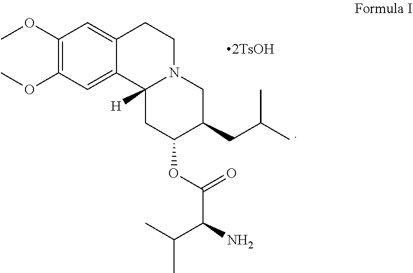 [SMILES: COc1cc2c(cc1OC)[C@H]1C[C@@H](OC(=O)[C@@H](N)C(C)C)[C@H](CC(C)C)CN1CC2]

In some embodiments, the present application provides processes of preparing a compound of Formula I:

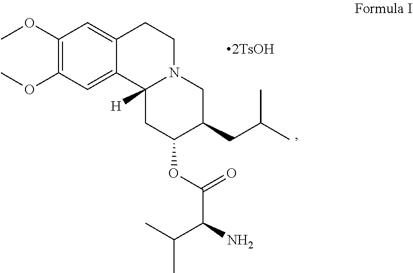 [SMILES: COc1cc2c(cc1OC)[C@H]1C[C@@H](OC(=O)[C@@H](N)C(C)C)[C@H](CC(C)C)CN1CC2]

comprising reacting a compound of Formula F8:

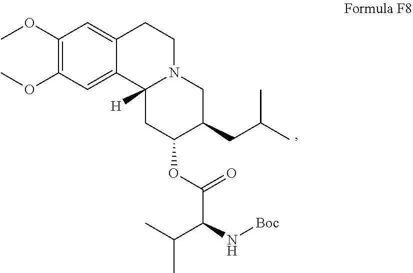 [SMILES: COc1cc2c(cc1OC)[C@H]1C[C@@H](OC(=O)[C@@H](NC(=O)OC(C)(C)C)C(C)C)[C@H](CC(C)C)CN1CC2]

with p-toluenesulfonic acid in a solvent comprising acetonitrile or isopropyl acetate, to afford the compound of Formula I.

In some embodiments, the present application provides processes of preparing a compound of Formula I:

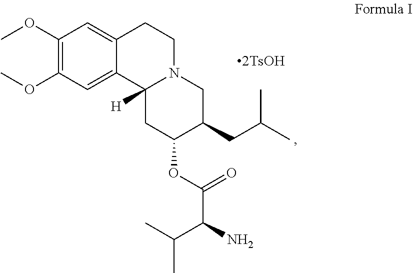 [SMILES: COc1cc2c(cc1OC)[C@H]1C[C@@H](OC(=O)[C@@H](N)C(C)C)[C@H](CC(C)C)CN1CC2]

comprising reacting a compound of Formula F8:

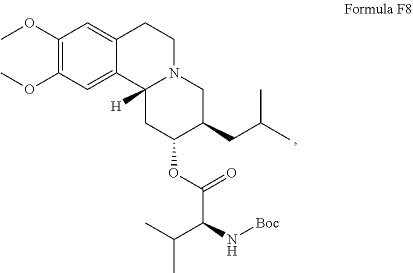 [SMILES: COc1cc2c(cc1OC)[C@H]1C[C@@H](OC(=O)[C@@H](NC(=O)OC(C)(C)C)C(C)C)[C@H](CC(C)C)CN1CC2]

with p-toluenesulfonic acid in a solvent comprising acetonitrile or isopropyl acetate, to afford a material comprising the compound of Formula I.

The processes may further comprise reacting a compound of Formula F6:

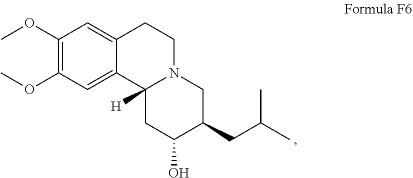 [SMILES: COc1cc2c(cc1OC)[C@H]1C[C@@H](O)[C@H](CC(C)C)CN1CC2]

with a carboxylic acid of Formula F7:

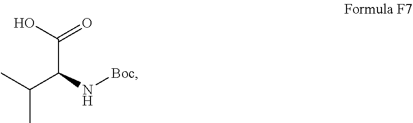

in a solvent to afford the compound of Formula F8.

The processes may further comprise reacting a compound of Formula F6-CSA:

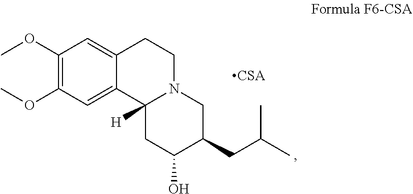 [SMILES: COc1cc2c(cc1OC)[C@H]1C[C@@H](O)[C@H](CC(C)C)CN1CC2]

with a base to afford a compound of Formula F6.

The processes may further comprise reacting a compound of Formula F5:

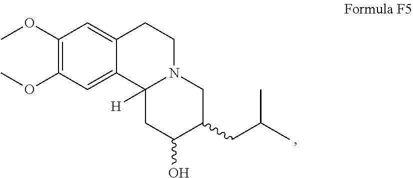 [SMILES: COc1cc2c(cc1OC)C1CC(O)C(CC(C)C)CN1CC2]

with (S)-(+)-camphorsulfonic acid (CSA) to afford a compound of Formula F6-CSA.

The processes may further comprise reacting a compound of Formula F4:

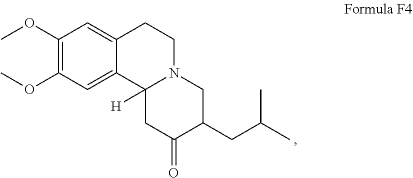 [SMILES: COc1cc2c(cc1OC)C1CC(=O)C(CC(C)C)CN1CC2]

with a reducing agent in a solvent comprising methyl tert-butyl ether (MTBE) and methanol to afford a compound of Formula F5.

The processes may further comprise reacting a compound of Formula F3:

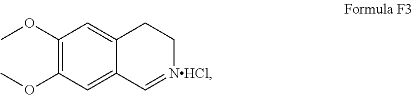

with a compound of Formula F2:

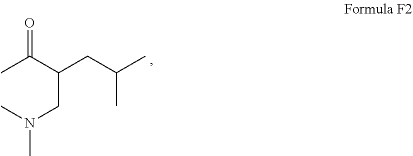

in a solvent comprising isopropanol (IPA) and water to afford a compound of Formula F4.

The processes may further comprise reacting a compound of Formula F1:

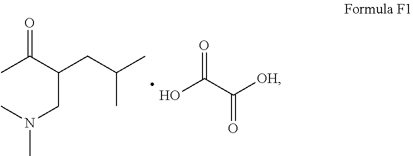

with a base, to afford a compound of Formula F2.

The processes may further comprise the step of crystallizing the compound of Formula I, comprising:

a) dissolving a material comprising the compound of Formula I in a solvent mixture comprising methanol and acetonitrile; and

b) crystallizing the compound of Formula I from the solvent mixture to afford a compound of

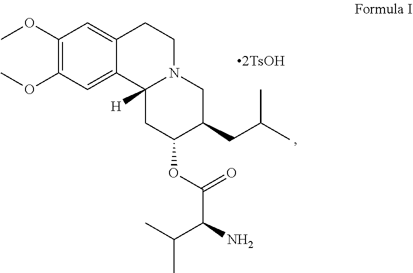 [SMILES: COc1cc2c(cc1OC)[C@H]1C[C@@H](OC(=O)[C@@H](N)C(C)C)[C@H](CC(C)C)CN1CC2]

The present application further provides processes of preparing a crystalline compound of Formula I, comprising:

a) dissolving a material comprising a compound of Formula I in a solvent mixture comprising methanol and acetonitrile; and

b) crystallizing the compound of Formula I from the solvent mixture to afford a crystalline compound of Formula I:

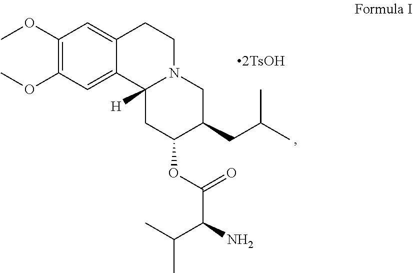 [SMILES: COc1cc2c(cc1OC)[C@H]1C[C@@H](OC(=O)[C@@H](N)C(C)C)[C@H](CC(C)C)CN1CC2]

The present application further provides processes of preparing a material comprising a compound of Formula I:

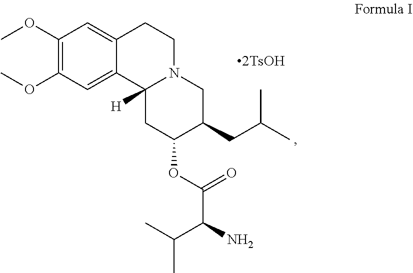 [SMILES: COc1cc2c(cc1OC)[C@H]1C[C@@H](OC(=O)[C@@H](N)C(C)C)[C@H](CC(C)C)CN1CC2]

comprising:

a) reacting a compound of Formula F6-CSA:

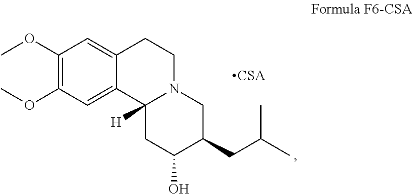 [SMILES: COc1cc2c(cc1OC)[C@H]1C[C@@H](O)[C@H](CC(C)C)CN1CC2]

with a base to afford a compound of Formula F6:

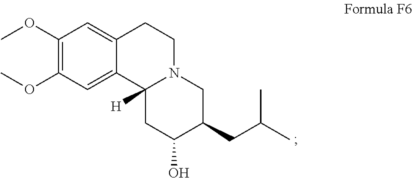 [SMILES: COc1cc2c(cc1OC)[C@H]1C[C@@H](O)[C@H](CC(C)C)CN1CC2]

b) reacting the compound of Formula F6 with a carboxylic acid of Formula F7:

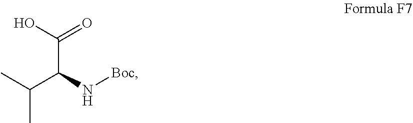

in a solvent to form a compound of Formula F8:

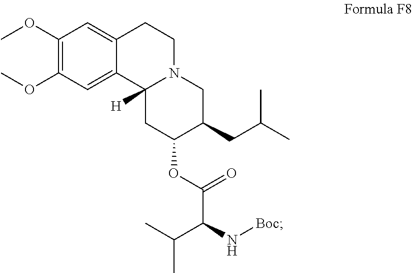 [SMILES: COc1cc2c(cc1OC)[C@H]1C[C@@H](OC(=O)[C@@H](NC(=O)OC(C)(C)C)C(C)C)[C@H](CC(C)C)CN1CC2]

c) reacting the compound of Formula F8 with p-toluenesulfonic acid in a solvent comprising acetonitrile or isopropyl acetate, to afford the material comprising the compound of Formula I.

The present application further provides processes of preparing a compound of Formula F6-CSA:

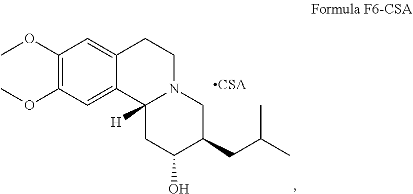 [SMILES: COc1cc2c(cc1OC)[C@H]1C[C@@H](O)[C@H](CC(C)C)CN1CC2]

comprising reacting a compound of Formula F5:

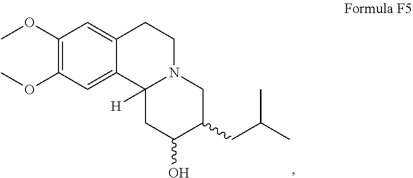 [SMILES: COc1cc2c(cc1OC)C1CC(O)C(CC(C)C)CN1CC2]

with (S)-(+)-camphorsulfonic acid (CSA), wherein the molar ratio of CSA to the compound of Formula F5 is between 0.7:1 and 0.9:1, to afford the compound of Formula F6-CSA.

The present application further provides processes of preparing a compound of Formula F5:

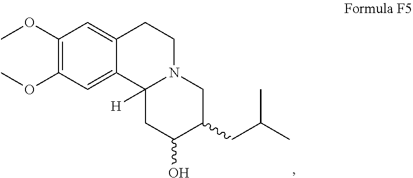 [SMILES: COc1cc2c(cc1OC)C1CC(O)C(CC(C)C)CN1CC2]

comprising reacting a compound of Formula F4:

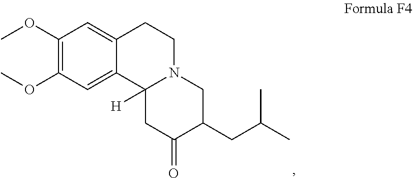 [SMILES: COc1cc2c(cc1OC)C1CC(=O)C(CC(C)C)CN1CC2]

with a reducing agent in a solvent comprising methyl tert-butyl ether (MTBE) and methanol, to afford the compound of Formula F5.

The present application further provides processes of preparing a compound of Formula F4:

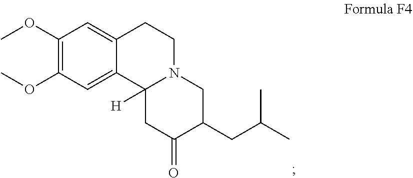 [SMILES: COc1cc2c(cc1OC)C1CC(=O)C(CC(C)C)CN1CC2]

comprising:

a) reacting a compound of Formula F1:

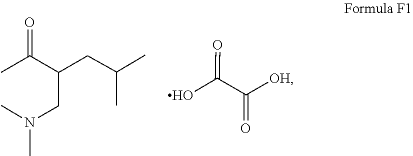

with a base to afford a compound of Formula F2:

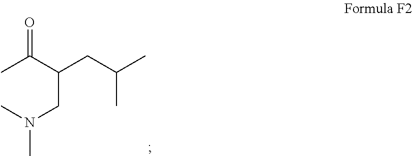

and

b) reacting the compound of Formula F2 with a compound of Formula F3:

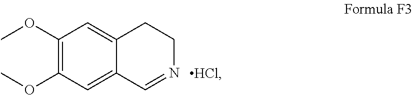

in the presence of sodium iodide in a solvent comprising isopropanol (IPA) and water, to afford the compound of Formula F4.

The present application further provides processes of preparing a crystalline compound of Formula I:

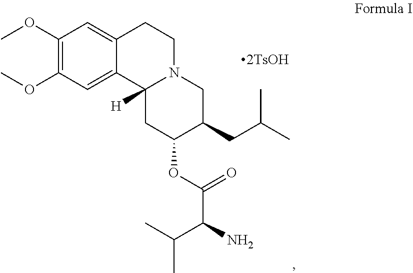 [SMILES: COc1cc2c(cc1OC)[C@H]1C[C@@H](OC(=O)[C@@H](N)C(C)C)[C@H](CC(C)C)CN1CC2]

comprising:

a) reacting a compound of Formula F1:

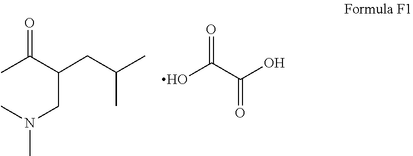

with a base, to afford a compound of Formula F2:

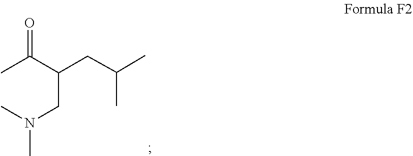

b) reacting a compound of Formula F3:

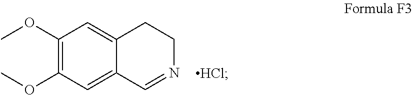

with the compound of Formula F2 in a solvent comprising isopropanol (IPA) and water to afford a compound of Formula F4:

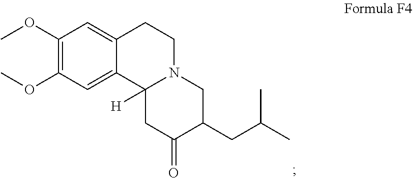 [SMILES: COc1cc2c(cc1OC)C1CC(=O)C(CC(C)C)CN1CC2]

c) reacting the compound of Formula F4 with a reducing agent in a solvent comprising methyl tert-butyl ether (MTBE) and methanol to afford a compound of Formula F5:

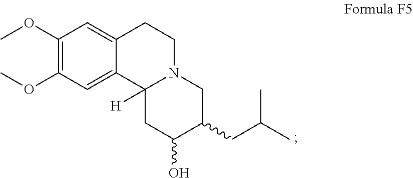 [SMILES: COc1cc2c(cc1OC)C1CC(O)C(CC(C)C)CN1CC2]

d) reacting the compound of Formula F5 with (S)-(+)-camphorsulfonic acid (CSA) to afford a compound of Formula F6-CSA:

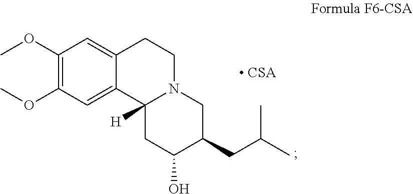 [SMILES: COc1cc2c(cc1OC)[C@H]1C[C@@H](O)[C@H](CC(C)C)CN1CC2]

e) reacting the compound of Formula F6-CSA with a base to afford a compound of Formula F6:

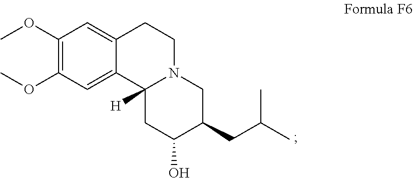 [SMILES: COc1cc2c(cc1OC)[C@H]1C[C@@H](O)[C@H](CC(C)C)CN1CC2]

f) reacting the compound of Formula F6 with a carboxylic acid of Formula F7:

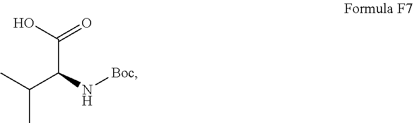

in a solvent to afford a product of Formula F8:

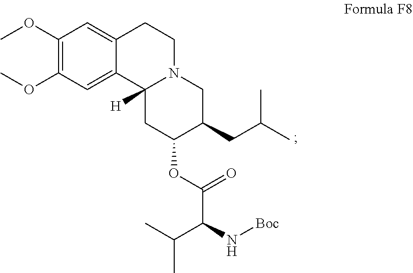 [SMILES: COc1cc2c(cc1OC)[C@H]1C[C@@H](OC(=O)[C@@H](NC(=O)OC(C)(C)C)C(C)C)[C@H](CC(C)C)CN1CC2]

g) reacting the product of Formula F8 with p-toluenesulfonic acid in acetonitrile or isopropyl acetate, to afford a material comprising the compound of Formula I; and

h) crystallizing the material comprising the compound of Formula I, comprising:
i) dissolving the material comprising the compound of Formula I in a solvent mixture comprising methanol and acetonitrile; and
ii) crystallizing the compound of Formula I from the solvent mixture to afford the compound of Formula I:

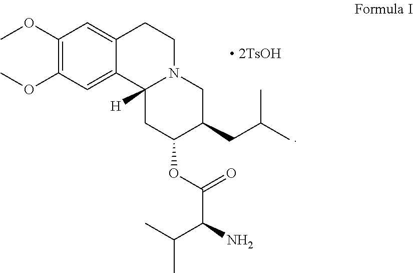 [SMILES: COc1cc2c(cc1OC)[C@H]1C[C@@H](OC(=O)[C@@H](N)C(C)C)[C@H](CC(C)C)CN1CC2]

In an alternate embodiment for the processes described above, step g) and step h) are as follows:

g) reacting the product of Formula F8 with p-toluenesulfonic acid in acetonitrile or isopropyl acetate, to afford a mixture comprising the compound of Formula I; and

h) crystallizing the compound of Formula I from the mixture to afford the compound of Formula I.

The present application further provides one or more of the processes as described herein, supra and infra, in Steps a) through h), either separately or together, that are useful in the preparation of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., the compound of Formula I).

The present application further provides the step of formulating the compound of Formula I to form a pharmaceutical composition. The present application further provides the step of formulating the compound of Formula I to form a pharmaceutical composition comprising: silicified microcrystalline cellulose; isomalt; hydroxypropyl methylcellulose; partially pregelatinized maize starch; and magnesium stearate. In some embodiments, the step of formulating comprises admixing the compound of Formula I with a pharmaceutically acceptable carrier and/or diluent to form a pharmaceutical composition comprising the compound of Formula I.

The present application further provides the step of formulating the crystalline form of the compound of Formula I to form a pharmaceutical composition. The present application further provides the step of formulating the crystalline form of the compound of Formula I to form a pharmaceutical composition comprising: silicified microcrystalline cellulose; isomalt; hydroxypropyl methylcellulose; partially pregelatinized maize starch; and magnesium stearate. In some embodiments, the step of formulating comprises admixing the crystalline form of the compound of Formula I with a pharmaceutically acceptable cater and/or diluent to form a pharmaceutical composition comprising the crystalline form of the compound of Formula I.

The present application further provides processes for preparing pharmaceutical compositions comprising: preparing a compound of Formula I according to the methods provided herein, supra and infra, and formulating the compound of Formula I with a pharmaceutically acceptable carrier and/or diluent.

The present application further provides processes for preparing the crystalline compound of Formula I. In some embodiments, the crystalline compound of Formula I is Form I as described in further details herein.

### BRIEF DESCRIPTION OF THE FIGURES

FIG. 1 shows an exemplary X-Ray Powder Diffraction (XRPD, Cu(Kα) radiation) pattern for a sample of crystalline Form I of (2R,3R,11 bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I) prepared according to Example 1.

FIG. 2 shows an exemplary Differential Scanning Calorimetry (DSC) for a sample of crystalline Form I of (2R,3R,11bR)-3-isobutyl-9,10-dimethoxy-2,3,4,6,7,111b-hexahydro-1H-pyrido[2,1-a]isoquinolin-2-yl (S)-2-amino-3-methylbutanoate di(4-methylbenzenesulfonate) (i.e., Compound of Formula I) prepared according to Example 1.

FIG. 3 shows the general preparation of 3-isobutyl-9,10-dimethoxy-3,4,6,7-tetrahydro-1H-pyrido[2,1-a]isoquinolin-2(11bH)-one (Compound of Formula F4) from 3-((dimethylamino)methyl)-5-methylhexan-2-one oxalate (Compound of Formula F1), referred to and described herein as Step A.

FIG. 4 shows the general preparation of 3-isobutyl-9,10-dimethoxy-2,3,4,6,7,11b-hexahydro-1H-pyrido[2,1-a]isoquinolin-2-ol (Compound of Formula F5) from 3-isobutyl-9,10-dimethoxy-3,4,6,7-tetrahydro-1H-pyrido[2,1-a]isoquinolin-2(11bH)-one (Compound of Formula F4), referred to and described herein as Step B; and the preparation of (2R,3R,11bR)-3-isobutyl-9,10-dimethoxy-2,3,4,6,7,11b-hexahydro-1H-pyrido[2,1-a]isoquinolin-2-ol (S)-(+)-camphorsulfonate (Compound of Formula F6-CSA) from 3-isobutyl-9,10-dimethoxy-2,3,4,6,7,11b-hexahydro-1H-pyrido[2,1-a]isoquinolin-2-ol (Compound of Formula F5), referred to and described herein as Step C.

FIG. 5 shows the general preparation of (2R,3R,11 bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (Formula I Intermediate) from (2R,3R,11 bR)-3-isobutyl-9,10-dimethoxy-2,3,4,6,7,11b-hexahydro-1H-pyrido[2,1-a]isoquinolin-2-ol (S)-(+)-camphorsulfonate (Compound of Formula F6-CSA), referred to and described herein as Step D.

FIG. 6 shows the general preparation of (2R,3R,11bR)-3-isobutyl-9,10-dimethoxy-2,3,4,6,7,11 b-hexahydro-1H-pyrido[2,1-a]isoquinolin-2-yl (S)-2-amino-3-methylbutanoate di(4-methylbenzenesulfonate) (Compound of Formula I) from (2R,3R,11 bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (Formula I Intermediate), referred to and described herein as Step E.

FIG. 7 shows the preparation of 3-isobutyl-9,10-dimethoxy-3,4,6,7-tetrahydro-1H-pyrido[2,1-a]isoquinolin-2(11 bH)-one (Compound of Formula F4) from 3-((dimethylamino)methyl)-5-methylhexan-2-one oxalate (Compound of Formula F1), referred to and described herein as Step A.

FIG. 8 shows the preparation of 3-isobutyl-9,10-dimethoxy-2,3,4,6,7,11b-hexahydro-1H-pyrido[2,1-a]isoquinolin-2-ol (Compound of Formula F5) from 3-isobutyl-9,10-dimethoxy-3,4,6,7-tetrahydro-1H-pyrido[2,1-a]isoquinolin-2(11bH)-one (Compound of Formula F4), referred to and described herein as Step B; and the preparation of (2R,3R,11bR)-3-isobutyl-9,10-dimethoxy-2,3,4,6,7,11b-hexahydro-1H-pyrido[2,1-a]isoquinolin-2-ol (S)-(+)-camphorsulfonate (Compound of Formula F6-CSA) from 3-isobutyl-9,10-dimethoxy-2,3,4,6,7,11b-hexahydro-1H-pyrido[2,1-a]isoquinolin-2-ol (Compound of Formula F5), referred to and described herein as Step C.

FIG. 9 shows the preparation of (2R,3R,11 bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (Formula I Intermediate) from (2R,3R,11 bR)-3-isobutyl-9,10-dimethoxy-2,3,4,6,7,11b-hexahydro-1H-pyrido[2,1-a]isoquinolin-2-ol (S)-(+)-camphorsulfonate (Compound of Formula F6-CSA), referred to and described herein as Step D.

FIG. 10 shows the preparation of (2R,3R,11bR)-3-isobutyl-9,10-dimethoxy-2,3,4,6,7,11 b-hexahydro-1H-pyrido[2,1-a]isoquinolin-2-yl (S)-2-amino-3-methylbutanoate di(4-methylbenzenesulfonate) (Compound of Formula I) from (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (Formula I Intermediate), referred to and described herein as Step E.

### DETAILED DESCRIPTION

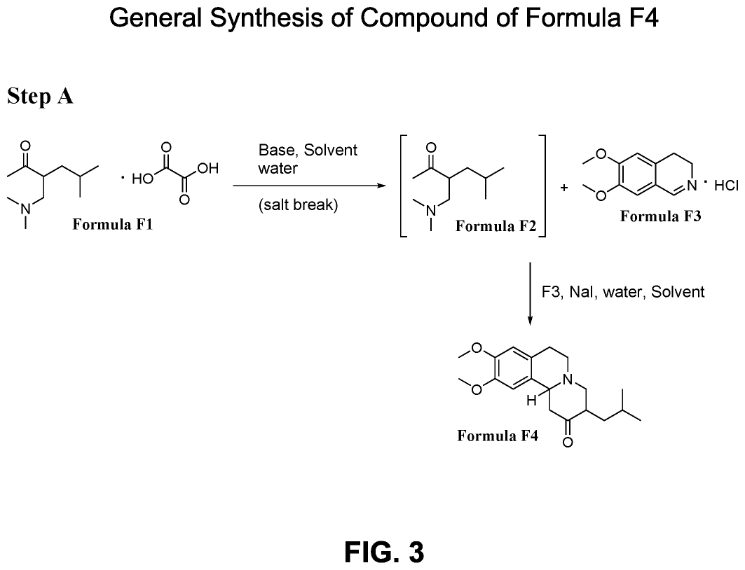

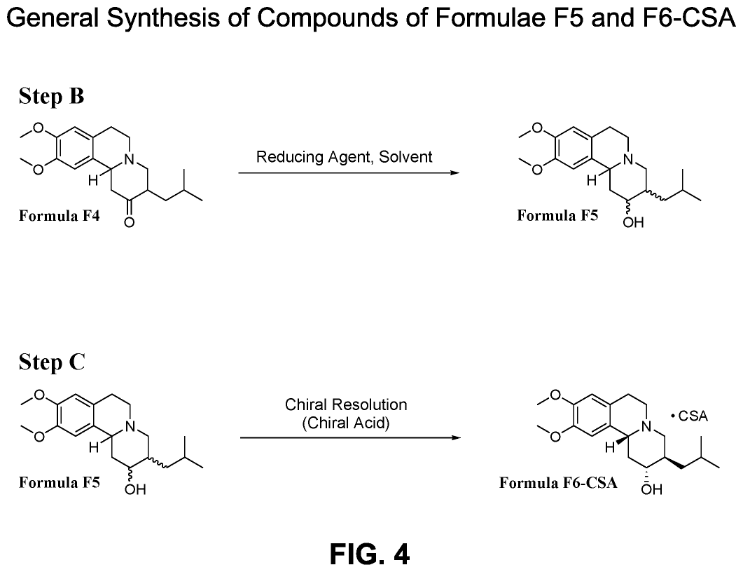

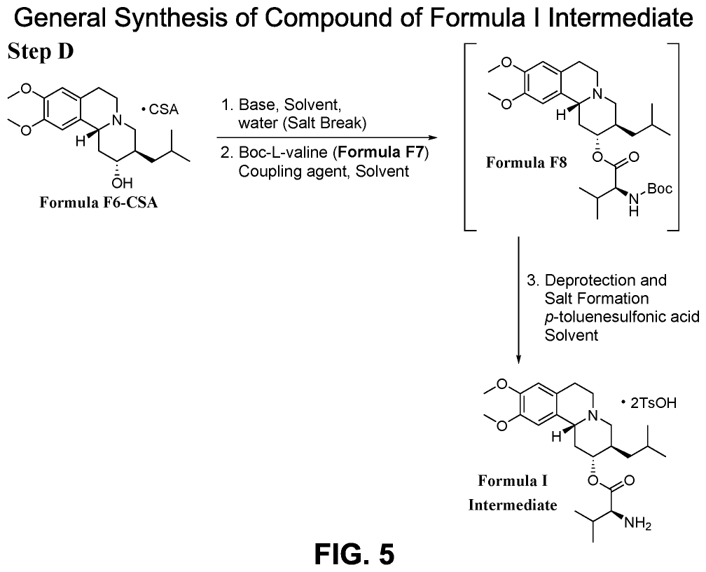

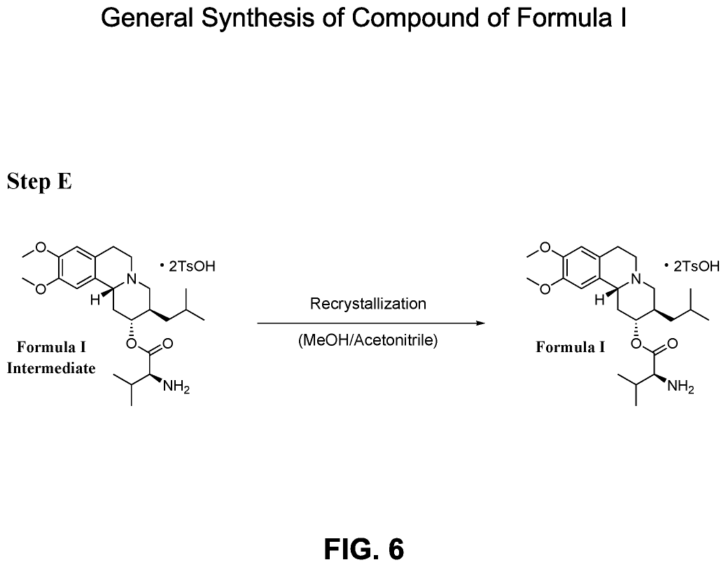

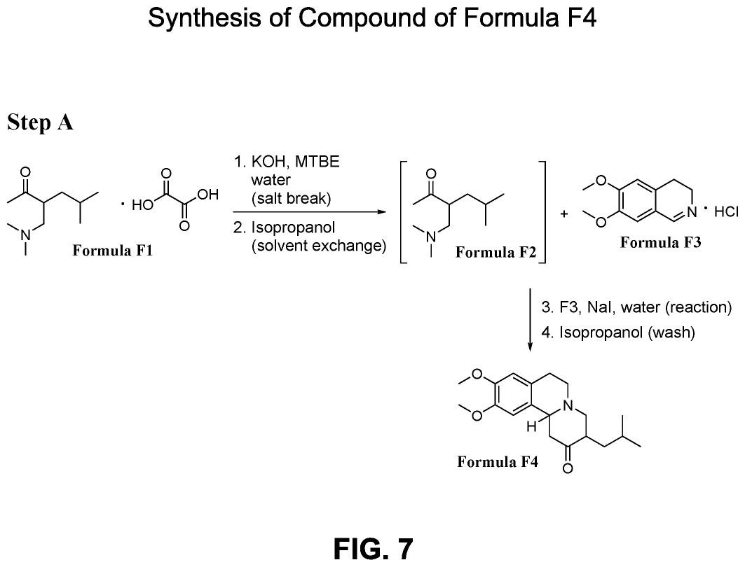

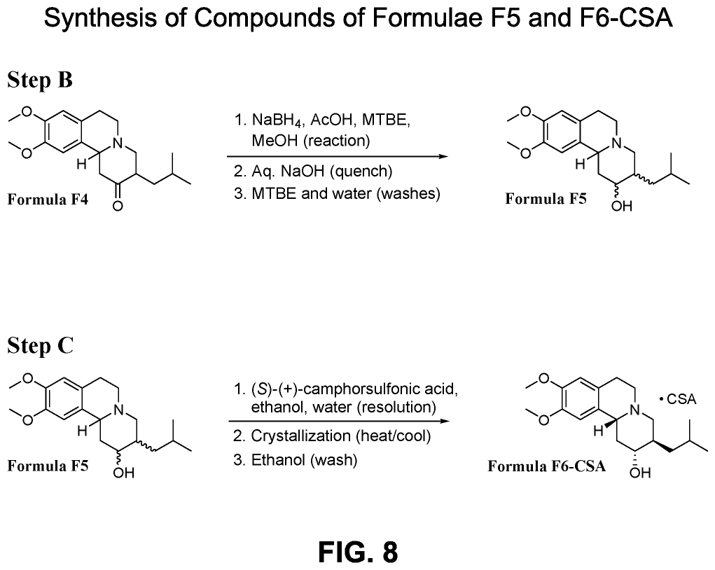

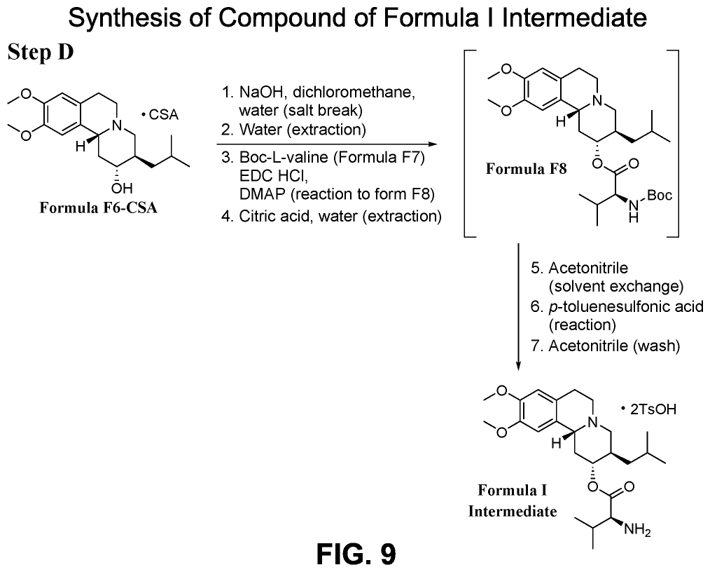

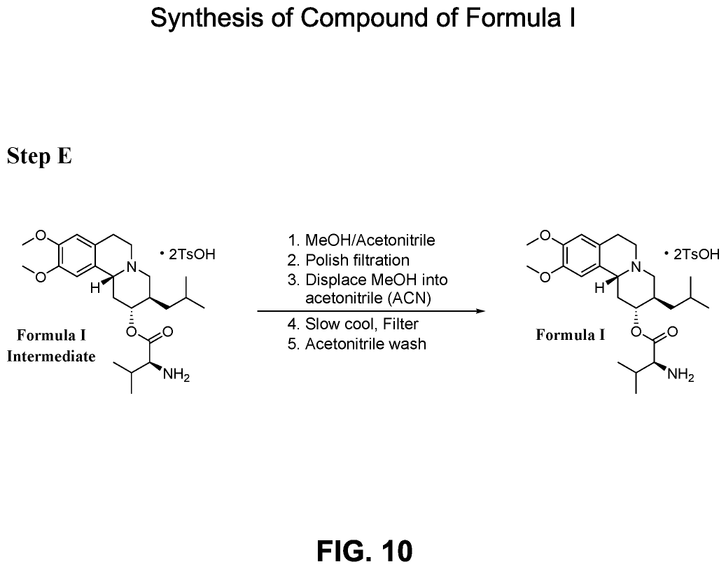

While certain of the process steps are illustrated in FIG. 3 and FIG. 7 (Step A), FIG. 4 and FIG. 8 (Steps B and C), FIG. 5 and FIG. 9 (Step D), and FIG. 6 and FIG. 10 (Step E), it is intended that the individual process steps may be claimed separately or in any combination (i.e., Steps A, B, C, D, and E may be claimed individually or in any combination thereof). It is not intended that the processes described herein be limited to an overall process having each and every step as shown in FIG. 3, FIG. 4, FIG. 5, FIG. 6, FIG. 7, FIG. 8, FIG. 9, and FIG. 10.

The synthesis of the compound of Formula I as described in U.S. Pat. No. 10,160,757 B2 (referred to as the “previous synthesis” herein, infra) is highly selective and robust, but opportunities existed for higher efficiency via minimization of process operations, truncating manufacturing time, and reducing the over-all process waste and environmental footprint. The processes disclosed herein were developed encompassing principles of Green Chemistry (see Warner, J. C.; Anastas, P. T.; Green Chemistry Theory and Practice. Oxford Univ. Press, 1998; Pharmaceutical Green Chemistry (Tucker, J. L., OPRD (2006), 10(2), 315-319; Tucker, J. L., OPRD (2010), 14(2), 328-331; Tucker, J. L.; Faul, M. M.; Nature (2016), 534(7605), 27-29). The processes described herein deliver greater efficiency and sustainability by reducing manufacturing operations, time of manufacture, and material use (waste, E-factor (Sheldon, R. A. Organic Synthesis Past, Present, and Future. Chem. Ind. (London) 1992)) while improving the overall process yield and reducing water use over the previous synthesis.

Corresponding Step A of the previous synthesis (described in U.S. Pat. No. 10,160,757) produces tetrabenazine (Formula F4) beginning with a salt break of amino ketone oxalate (Formula F1) using aqueous NaOH/n-heptane, followed by partition and a water wash to deliver the freebase in n-heptane. The freebase solution is then combined with the HCl salt of dihydroisoquinoline (Formula F3) in water. The biphasic mixture is stirred at 30-40° C. for at least 48 hours until less than 10% of dihydroisoquinoline (Formula F3) remained. The solid was filtered and dried under vacuum to provide the compound of Formula F4 in yields of 79% and 86% (U.S. Pat. No. 10,160,757, Examples 1, A and Al respectively).

Although Step A of the previous synthesis was performed using low solvent volume and exhibited excellent atom economy, the process suffers from extended stir time at 30-40° C. due to the bi-phasic conditions and subsequent restrictions of mass transfer. In the new iteration, as described herein, the solvent mixture was designed to enhance reaction homogeneity to accelerate the reaction kinetics and reduce overall waste. In contrast to the previous synthesis, Step A herein also uses sodium iodide in an initially homogeneous reaction with a smaller overall volume of isopropanol (IPA)/water rather than the previously used water/heptanes. The acceleration of the overall reaction kinetics achieves >95% reaction conversion (remaining Formula F3<5%) in 24 hours vs. the previous synthesis which required at least 48 hours until less than 10% of dihydroisoquinoline (Formula F3) remained. The atom efficiency remains high and as the reaction progresses with tetrabenazine (Formula F4) precipitating directly from the reaction mixture. Upon completion, the slurry is simply cooled, filtered and washed with IPA before being dried under vacuum.

Accordingly, one improvement for Step A, is the reduction in reaction/plant time compared to the previous synthesis. In addition, Step A now also provides tetrabenazine with >99% purity and 88% yield.

Step B, as described herein (FIG. 4 and FIG. 8), is a sodium borohydride (NaBH₄) mediated reduction of the compound of Formula F4 to provide a mixture of four Formula F5 isomers (i.e., carbonyl to 2°±alcohol). The four Formula F5 isomers are shown below:

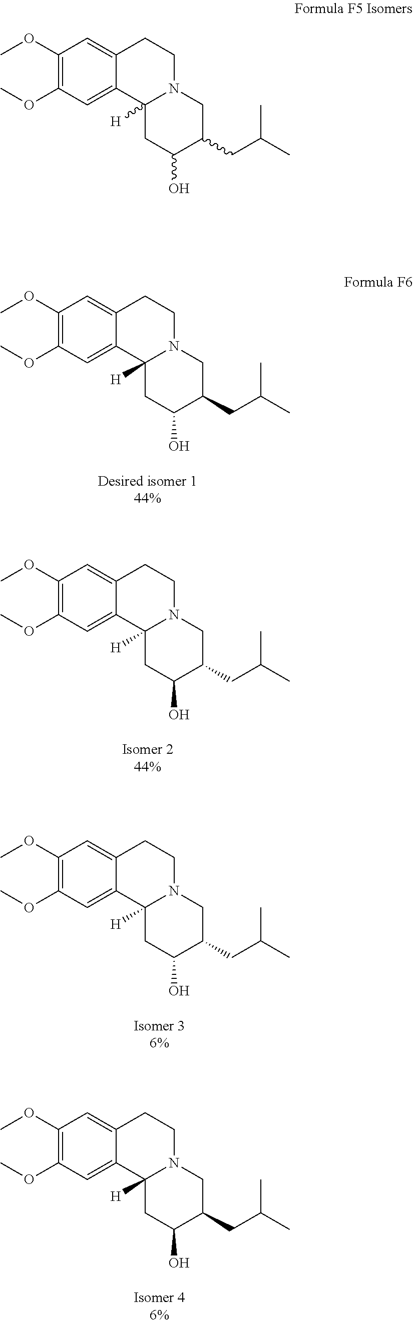

The previous synthesis (described in U.S. Pat. No. 10,160,757) used 1.2 eq. NaBH₄, 1.0 eq. lithium chloride (LiCI) and 1.1 eq. acetic acid (AcOH) in 19 volumes of ethanol (EtOH) and 2.1 volumes of methylene chloride (CH₂Cl₂) at −10±5° C. (i.e., −15° C. to −5° C.). During development, it was discovered that LiCl, AcOH, and temperature were shown to positively impact selectivity for the desired isomer 1 (i.e., Formula F6). Upon reaction completion, the mixture was warmed to 25° C. and quenched with saturated ammonium chloride. After stirring, the EtOH was distilled off under vacuum and fresh CH₂Cl₂ added to aid in partition during work-up. The pH was adjusted with aqueous NaOH before the lower organic layer was separated. The aqueous was extracted a second time with CH₂Cl₂ before the combined organic layers were washed with water. The CH₂Cl₂ solution was then displaced under vacuum (put and take) to 3 volumes (L/kg) total in isopropyl acetate (i-PrOAc). The mixture was heated to dissolution and upon cooling to 65° C., a suspension formed which was cooled further to 20° C. After stirring, the suspension was filtered, washed with i-PrOAc and dried under vacuum. The yields for the two examples were 86% and 85% respectively (U.S. Pat. No. 10,160,757, Example 1B).

Although the previous procedure was robust, the disadvantages encompassed extensive operations to manage water-miscible solvents and an excess of material use overall. Ethanol was used to solubilize LiCI and to transfer NaBH₄ as a slurry. This led to the requirement to distill off the EtOH and CH₂Cl₂ prior to extractive isolation of product after quench. In addition, the EtOH reacted with NaBH₄ during slurry preparation before charging, thus limiting the hold-time possible prior to use and exacerbating hydrogen gas evolution in a secondary reactor system. After work-up, it was also necessary to spend significant energy and time to displace CH₂Cl₂ into i-PrOAc for effective crystallization and isolation.

The reaction solvent volume, diversity of solvents used, and extensive number of operations engendered this step with the highest waste production of all the chemical steps to manufacture Valbenazine ditosylate (Formula I) and led to excessive personnel and plant time.

Step B, as described herein (see FIG. 4 and FIG. 8), is performed by first adding the compound of Formula F4 to methyl tert-butyl ether (MTBE) and methanol (MeOH) along with 0.9 eq. of AcOH. To this mixture, 1.7 eq. NaBH₄ is then added (as a slurry) in MTBE to provide a complete reaction in 4 hours at 20-30° C. This solvent volume and ratio combined with the ambient temperature provided ideal solubility and kinetics for a safe and reliable reduction to occur. Delivering the NaBH₄ in MTBE (rather than reactive EtOH) prevented undesired hydrogen off gassing and the ambient temperature eliminated the need for energy intensive cooling of the vessel. The mixture also eliminated the need for LiCl, which provided no additional desired selectivity relative to AcOH alone in this solvent system. At the end of the reaction, the slurry is quenched with 1M aqueous sodium hydroxide (NaOH) and heated to 45-50° C. for 3 hours to decompose residual boron-amine complexes. The slurry is then cooled to 15° C., stirred for 1-2 hours and isolated directly via filtration. The solid product is then washed with water followed by MTBE and dried in a vacuum oven to provide 80% yield of a compound of Formula F5. Overall, the process simplification enhances efficiency requiring only 4 days at scale vs. the original 8 days, and the waste has been significantly reduced.

Step C is a (+)-(1S)-camphor-10-sulfonic acid (CSA) salt resolution of a single diastereomer of dihydrotetrabenazine.

The previous synthesis (described in U.S. Pat. No. 10,160,757) involved forming a suspension of racemic F5 and (+)-(1S)-camphor-10-sulfonic acid (1.0 equivalent) in ethanol:water (19:1, v/v). The mixture was heated to 75° C. to obtain a solution before cooling to 53° C.±2° C. (i.e., 51° C.-55° C.) and held until crystallization occurs or adding a seed crystal if nucleation did not occur. The ripening slurry is stirred before cooling to 25° C.±5° C. over 14 hours at a rate of about 2° C. per hour. The slurry was filtered, washed with ethanol, and dried under vacuum. The two reported yields for the previous synthesis were both 38% (U.S. Pat. No. 10,160,757, Example 1C), and the material had >99% diastereoselectivity.

Step C, as disclosed herein (see FIG. 4 and FIG. 8), included experiments evaluating temperature, solvent ratio, and volume, as well as stoichiometry of CSA to arrive at a process that significantly reduces the overall waste of Step C. The compound of Formula F5 is combined with 0.825 equivalents CSA, EtOH and water. The mixture is heated to dissolution at 70° C., cooled to 50-55° C. at which temperature the initial crystallization occurs. The slurry is then cooled to 20° C. at 3° C. per hour before the product is filtered, washed with 2 volumes EtOH and dried under vacuum. The recovery is 37% yield with >99% diastereomeric purity. While the time, operations, and yield remain similar with the previous synthesis, the waste generated and capacity required (this previously was the high-point for process volume/lowest concentration step) have been optimized, enabling higher operational and process waste efficiency and providing 56% additional isolated compound of Formula F6-CSA vs. the previous synthesis from a same volume vessel.

Step D of the previous synthesis (described in U.S. Pat. No. 10,160,757) can be viewed as telescoping four distinct, chemical processes; 1) breaking of the F6-CSA salt, 2) coupling of freebase F6 to F7,3) Boc deprotection of intermediate of Formula F8, and 3) isolation of the di-HCl salt of Valbenazine. Step D of the previous synthesis is operationally intense. The first step requires combining 1M NaOH with F6-CSA in CH₂Cl₂ to break the camsylate salt. The mixture is stirred, settled, and separated. The lower organic layer is washed with water to provide the free base of F6, in 6 volumes total of CH₂Cl₂. Boc-L-valine (1.2 eq.) and dimethylaminopyridine (DMAP, 0.3 eq.) are added to the freebase solution before cooling to approximately 0° C. To the mixture was added N-(3-dimethylaminopropyl)-N′-ethylcarbodiimide hydrochloride keeping the temperature 0° C.±5° C. The mixture is stirred for at least 3 hours, and after 5 hours the reaction is typically complete. The reaction is quenched with aqueous citric acid and washed with water to afford the coupled product as a CH₂Cl₂ solution. The solution is cooled to 5-10° C. and 5 eq. of 4M HCl in dioxane is added for Boc deprotection. The reaction is warmed to 20-30° C. and stirred for at least 12 hours. Upon completion, aqueous sodium bicarbonate is used to adjust the pH and the product is then extracted into the organic layer and washed with water. The organic layer is then displaced under vacuum into acetonitrile (ACN), cooled to 5-15° C. and 2.1 eq. of 3.7 M HCl in IPA is added to generate the di-HCl salt. Ethyl acetate is then charged, and the mixture warmed to 45-55° C. before seeding. Additional EtOAc is then added and the slurry warmed to 65-75° C. and stirred 1 hour before cooling to 20-30° C. and granulating an additional 3 hours. Filtration and wash with EtOAc then provides Valbenazine di-HCl in a 79% yield (U.S. Pat. No. 10,160,757, Example 1E).

In step D, as provided herein (see FIG. 5 and FIG. 9), the organic layer of the crude F8 product is displaced in ACN which was then used as the solvent for Boc deprotection and isolation of the Formula I intermediate.

The compound of Formula I is generated in ACN in step D via Boc deprotection using p-toluene sulfonic acid (p-TSA or TsOH) directly, rather than HCl/dioxane as in the previous synthesis. The use of a single reagent (TsOH) serves as both the acid catalyst for the Boc deprotection and the isolation counter ion to procure the compound of Formula I (di-tosylate salt) directly and therefore eliminating the requirement of isolating Valbenazine di-HCl as an intermediate as described in the previous synthesis. The new and improved process reduces process steps, time (i.e., plant and personnel), and waste; and is also extremely atom economical. During deprotection, the di-TsOH salt of Valbenazine (Formula I) crystallizes directly from the reaction mixture to produce an intermediate compound of Formula I of high purity, thus eliminating the additional operations to isolate the HCl salt intermediate, use of the toxic HCl/dioxane reagent, the need to quench the acidic mixture while off-gassing CO₂ and the significant time and energy required to distill under vacuum from ACN into EtOAc employed in the previous synthesis.

The deprotection of Step D, as described herein, is carried out by simply adding 2.1 eq. p-TSA to intermediate of Formula F8 and warming. The resulting intermediate Valbenazine ditosylate (Formula I) is filtered, washed and dried to provide 86% yield in >99% purity. Overall, this step requires only 4 days vs. the original 8 days processing due to the elimination of many operations while concomitantly improving the recovery by 10% and reducing waste.

Step E of the previous synthesis (described in U.S. Pat. No. 10,160,757) entailed first a salt break of Valbenazine di-HCl in CH₂Cl₂ and aqueous sodium bicarbonate, followed by displacement into ACN and polish filtration of the solution. 2.0 equivalents of TsOH were then dissolved in ACN, and the TsOH solution was added to the solution of Valbenazine (as the free base) through a polish filter. The solution was added at elevated temperature at a proscribed rate and held to ensure polymorph and particle size control. Following the hold, the slurry was cooled to 25° C., filtered, washed with ACN, and dried to give the crystalline compound of Formula I in yields of 92.8% and 88% (U.S. Pat. No. 10,160,757, Examples iF and 1F1).

Step E, as provided herein (see FIG. 6 and FIG. 10), is a re-crystallization of the intermediate compound of Formula I produced in Step D. The process begins with dissolution of the intermediate compound of Formula I in MeOH and ACN followed by a polish filtration into a second vessel. The crystallization is then driven by removal of MeOH via constant volume distillation while adding 4 volumes of ACN. The batch is seeded after a portion of the solvent has been removed and after completion of the solvent exchange, 1 volume of ACN is used to rinse the vessel before the suspension is cooled, filtered, washed and dried. The dry product is obtained with a 97% yield and having quality consistent of the previous synthesis. Step E also provides control of attributes such as particle size and crystal morphology, and the implementation of a robust and redundant impurity control strategy.

Due to the presence of TsOH and methanol in the initial dissolution prior to crystallization of the compound of Formula I, formation of a potential genotoxic impurity (i.e. methyl toluenesulfonate) was of concern. It was determined that eliminating excess (free) TsOH in the methanol/ACN solution prevented formation of the ester below 50° C. In addition, when the solution was spiked with 0.1 equivalents excess TsOH to challenge the system, the ester (i.e. methyl toluenesulfonate) did form at levels up to 2700 ppm in solution. However even at this level, the crystallization proved effective at purging the ester to <5 ppm in the isolated Valbenazine ditosylate (Formula I). Because it is preferential to avoid formation of the methyl toluenesulfonate altogether, a simple and sensitive HPLC test was established upstream on the crude Valbenazine di-TsOH (obtained from step D) isolated solid to detect any excess TsOH to allow reprocessing (if necessary) prior to dissolution in methanol at Step E. See Example 2.

After adoption of the processes provided herein, metrics associated with the processes have demonstrated the positive impact compared to the previous synthesis, from an environmental, economic, and strategic business standpoint. In addition to waste and cost reductions, the processes provided herein allow for quick production of batches of Valbenazine ditosylate (i.e., the compound of Formula F1) to rapidly adapt to patient needs.

Accordingly, the present application provides processes for preparing a compound of Formula I:

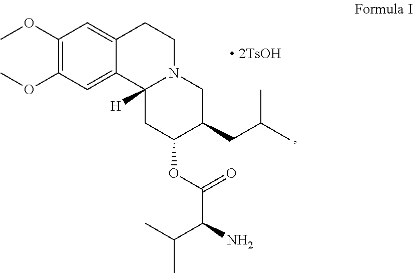 [SMILES: COc1cc2c(cc1OC)[C@H]1C[C@@H](OC(=O)[C@@H](N)C(C)C)[C@H](CC(C)C)CN1CC2]

comprising reacting a compound of Formula F8:

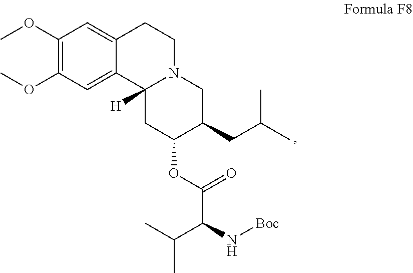 [SMILES: COc1cc2c(cc1OC)[C@H]1C[C@@H](OC(=O)[C@@H](NC(=O)OC(C)(C)C)C(C)C)[C@H](CC(C)C)CN1CC2]

with p-toluenesulfonic acid in a solvent comprising acetonitrile or isopropyl acetate, to afford a material comprising the compound of Formula I.

In some embodiments, the present application also provides processes of preparing a compound of Formula I:

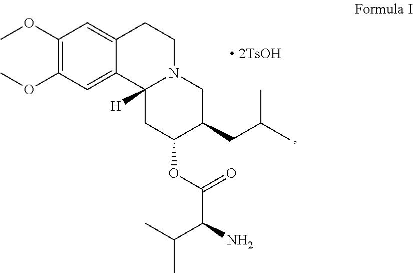 [SMILES: COc1cc2c(cc1OC)[C@H]1C[C@@H](OC(=O)[C@@H](N)C(C)C)[C@H](CC(C)C)CN1CC2]

comprising reacting a compound of Formula F8:

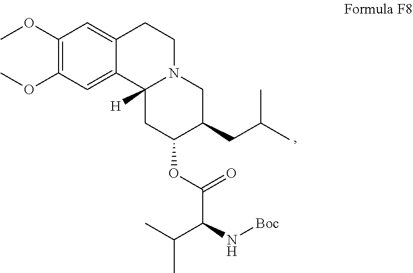 [SMILES: COc1cc2c(cc1OC)[C@H]1C[C@@H](OC(=O)[C@@H](NC(=O)OC(C)(C)C)C(C)C)[C@H](CC(C)C)CN1CC2]

with p-toluenesulfonic acid in a solvent comprising acetonitrile or isopropyl acetate, to afford the compound of Formula I.

In some embodiments, the solvent does not comprise acetonitrile. In some embodiments, the solvent does not comprise isopropyl acetate. In some embodiments, the solvent does not comprise acetonitrile or isopropyl acetate.

In some embodiments, the solvent is petroleum ether, pentane, hexane(s), heptane, octane, isooctane, cyclopentane, cyclohexane, methylcyclohexane, benzene, toluene, xylene, tetralin, cumene, dichloromethane (DCM), 1,2-dichloroethane, 1,1-dichloroethene, 1,2-dichloroethene, chloroform, trichloroethane, trichloroethene, carbon tetrachloride, chlorobenzene, trifluoromethylbenzene, methanol, ethanol, isopropanol (IPA), 1-propanol, 1-butanol, 2-butanol, t-butanol, 3-methyl-1-butanol, 1-pentanol, 2-methoxyethanol, 2-ethoxyethanol, ethyleneglycol, diethyl ether, diisopropyl ether, methyl t-butyl ether (MTBE), di phenyl ether, 1,2-dimethoxyethane, bi(2-methoxyethyl)ether, 1,1-dimethoxymethane, 2,2-dimethoxypropane, anisole, acetone, butanone, methyl ethyl ketone (MEK), methyl isopropyl ketone, methyl butyl ketone, methyl isobutyl ketone (MIBK), methyl acetate, ethyl formate, ethyl acetate, propyl acetate, isopropyl acetate, isobutyl acetate, butyl acetate, ethylene carbonate, propylene carbonate, formamide, N,N-dimethylformamide (DMF), N,N-dimethylacetamide, acetonitrile (ACN), dimethyl sulfoxide (DMSO), sulfolane, nitromethane, nitrobenzene, N-methyl pyrrolidone, 2-methyl tetrahydrofuran, tetrahydrofuran (THF), dioxane, pyridine, formic acid, acetic acid, trichloroacetic acid, trifluoroacetic acid, hexamethylphosphoramide, carbon sulfide, water; or a mixture thereof. In some embodiments, the solvent is isopropanol. In some embodiments, the solvent is a mixture of dichloromethane and acetonitrile. In some embodiments, the solvent is acetonitrile. In some embodiments, the solvent is isopropyl acetate.

In some embodiments, the reacting of the compound of Formula F8 with p-toluenesulfonic acid is carried out at an elevated temperature. In some embodiments, the reacting of the compound of Formula F8 with p-toluenesulfonic acid is carried out at a temperature of about 35° C. to about 80° C. In some embodiments, the reacting of the compound of Formula F8 with p-toluenesulfonic acid is carried out at a temperature of about 35° C. to about 75° C. In some embodiments, the reacting of the compound of Formula F8 with p-toluenesulfonic acid is carried out at a temperature of about 40° C. to about 75° C.

In some embodiments, the reacting of the compound of Formula F8 with p-toluenesulfonic acid is carried out at a temperature of about 45° C. to about 75° C. In some embodiments, the reacting of the compound of Formula F8 with p-toluenesulfonic acid is carried out at a temperature of about 50° C. to about 75° C. In some embodiments, the reacting of the compound of Formula F8 with p-toluenesulfonic acid is carried out at a temperature of about 55° C. to about 75° C. In some embodiments, the reacting of the compound of Formula F8 with p-toluenesulfonic acid is carried out at a temperature of about 60° C. to about 70° C. In some embodiments, wherein the reacting of the compound of Formula F8 with p-toluenesulfonic acid is carried out at a temperature of about 62° C. to about 68° C. In some embodiments, wherein the reacting of the compound of Formula F8 with p-toluenesulfonic acid is carried out at a temperature of about 63° C. to about 67° C. In some embodiments, wherein the reacting of the compound of Formula F8 with p-toluenesulfonic acid is carried out at a temperature of about 64° C. to about 66° C. In some embodiments, the reacting of the compound of Formula F8 with p-toluenesulfonic acid is carried out at a temperature of about 65° C.

In some embodiments, the reacting of the compound of Formula F8 with p-toluenesulfonic acid is carried out over a period sufficient to reduce the presence of the compound of Formula F8 to at least 10% as determined by HPLC. In some embodiments, the reacting of the compound of Formula F8 with p-toluenesulfonic acid is carried out over a period sufficient to reduce the presence of the compound of Formula F8 to at least 5% as determined by HPLC. In some embodiments, the reacting of the compound of Formula F8 with p-toluenesulfonic acid is carried out over a period sufficient to reduce the presence of the compound of Formula F8 to at least 4% as determined by HPLC. In some embodiments, the reacting of the compound of Formula F8 with p-toluenesulfonic acid is carried out over a period sufficient to reduce the presence of the compound of Formula F8 to at least 3% as determined by HPLC. In some embodiments, the reacting of the compound of Formula F8 with p-toluenesulfonic acid is carried out over a period sufficient to reduce the presence of the compound of Formula F8 to at least 2% as determined by HPLC. In some embodiments, the reacting of the compound of Formula F8 with p-toluenesulfonic acid is carried out over a period of about 6 hours to about 18 hours. In some embodiments, the reacting of the compound of Formula F8 with p-toluenesulfonic acid is carried out over a period of about 8 hours to about 16 hours. In some embodiments, the reacting of the compound of Formula F8 with p-toluenesulfonic acid is carried out over a period of about 10 hours to about 14 hours. In some embodiments, the reacting of the compound of Formula F8 with p-toluenesulfonic acid is carried out over a period of about 12 hours. In some embodiments, after the reacting of the compound of Formula F8 with p-toluenesulfonic acid the process further comprises cooling to a temperature of about 10° C. to about 30° C. In some embodiments, after the reacting of the compound of Formula F8 with p-toluenesulfonic acid the process further comprises cooling to a temperature of about 15° C. to about 25° C.

In some embodiments, after the reacting of the compound of Formula F8 with p-toluenesulfonic acid the process further comprises cooling to a temperature of about 18° C. to about 22° C. In some embodiments, the temperature is maintained for about 1 hour to about 3 hours. In some embodiments, the temperature is maintained for about 1.5 hours to about 2.5 hours. In some embodiments, the temperature is maintained for about 1.8 hours to about 2.2 hours. In some embodiments, the temperature is maintained with stirring. In some embodiments, the reacting of the compound of Formula F8 with p-toluenesulfonic acid in a solvent affords a reaction mixture. In some embodiments, the reaction mixture is further cooled to about 10° C. to about 30° C. In some embodiments, the reaction mixture is further cooled to about 15° C. to about 25° C. In some embodiments, the reaction mixture is further cooled to about 18° C. to about 22°±C. In some embodiments, the reaction mixture is further cooled and stirred for about 1 hours to about 3 hours. In some embodiments, the reaction mixture is further cooled and stirred for about 1.5 hours to about 2.5 hours. In some embodiments, the reaction mixture is further cooled and stirred for about 2 hours. In some embodiments, the reaction mixture is further cooled to about 18° C. to about 22° C. and stirred for about 1.5 hours to about 2.5 hours. In some embodiments, the reaction mixture is further cooled to about 20° C. and stirred for about 2 hours.

In some embodiments, the ratio of p-toluenesulfonic acid to the compound of Formula F8 ranges from about 1.9:1 to about 2.3:1 molar equivalents. In some embodiments, the ratio of p-toluenesulfonic acid to the compound of Formula F8 ranges from about 2.0:1 to about 2.2:1 molar equivalents. In some embodiments, the ratio of p-toluenesulfonic acid to the compound of Formula F8 is about 2.1:1 molar equivalents. In some embodiments, excess p-toluenesulfonic acid is undetectable in the material comprising the compound of Formula I. In some embodiments, excess p-toluenesulfonic acid is undetectable in the material comprising the compound of Formula I as determined by HPLC.

In some embodiments, the compound of Formula I is isolated by washing with acetonitrile and drying at an elevated temperature under vacuum. In some embodiments, the compound of Formula I is dried at about 50° C. under vacuum for no less than about 12 hours.

In some embodiments, the reacting a compound of Formula F8 with p-toluenesulfonic acid, further comprises the step of formulating the compound of Formula I to form a pharmaceutical composition. In some embodiments, the step of formulating comprises admixing the compound of Formula I with a pharmaceutical excipient, a pharmaceutically acceptable carrier, and/or diluent.

In some embodiments, any unacceptable excess p-toluenesulfonic acid detected in the material comprising the compound of Formula I is removed. In some embodiments, any unacceptable excess p-toluenesulfonic acid detected in the material comprising the compound of Formula I is removed by recrystallizing the material containing the unacceptable excess p-toluenesulfonic acid in the presence of a recrystallizing solvent. In some embodiments, the recrystallizing solvent comprises acetonitrile. In some embodiments, the recrystallizing solvent is acetonitrile.

In some embodiments, the compound of Formula F8 is prepared by a process comprising reacting a compound of Formula F6:

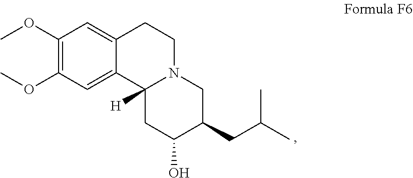 [SMILES: COc1cc2c(cc1OC)[C@H]1C[C@@H](O)[C@H](CC(C)C)CN1CC2]

with a carboxylic acid of Formula F7:

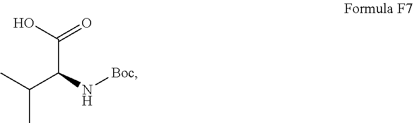

in a solvent.

In some embodiments, the reacting of the compound of Formula F6 with a carboxylic acid of Formula F7 is performed in a solvent which is a hydrocarbon, chlorinated hydrocarbon, alcohol, ether, ester, carbonate, amide, nitrile, sulfoxide, sulfone, nitro compound, heteroarene, heterocycle, water, or a mixture thereof. In some embodiments, the solvent is a chlorinated hydrocarbon solvent. In some embodiments, the solvent is dichloromethane. In some embodiments, the solvent is an ether. In some embodiments, the solvent is a cycloalkyl ether. In some embodiments, the solvent is 2-methyltetrahydroforan (MeTHF).

In some embodiments, the reacting of the compound of Formula F6 with a carboxylic acid of Formula F7 is performed in a solvent comprising a halogenated hydrocarbon solvent. In some embodiments, said halogenated hydrocarbon solvent is dichloromethane.

In some embodiments, the reacting of the compound of Formula F6 with the carboxylic acid of Formula F7 is carried out in the presence of a coupling reagent. In some embodiments, the reacting of the compound of Formula F6 with the carboxylic acid of Formula F7 is carried out in the presence of a base. In some embodiments, the reacting of the compound of Formula F6 with the carboxylic acid of Formula F7 is carried out in the presence of a catalytic base. In some embodiments, the reacting of the compound of Formula F6 with the carboxylic acid of Formula F7 is carried out in the presence of a coupling reagent and a base. In some embodiments, the reacting of the compound of Formula F6 with the carboxylic acid of Formula F7 is carried out in the presence of a coupling reagent and a catalytic base.

In some embodiments, the coupling reagent is a carbodiimide, 1,1′-carbonyldiimidazole (CDI), bis(2-oxo-3-oxazolidinyl)phosphinic chloride (BOP—Cl), hexafluorophosphate (BOP reagent), PCh, PCls, or 1-propanephosphonic acid cyclic anhydride. In some embodiments, the coupling reagent is N-(3-dimethylaminopropyl)-N-ethylcarbodiimide (EDC or EDCI), N-(3-dimethylaminopropyl)-N-ethylcarbodiimide hydrochloride (EDC hydrochloride), 1-[3-(dimethylamino)propyl]-3-ethylcarbodiimide methiodide (EDC methiodide), 1-cyclohexyl-3-(2-morpholinoethyl)carbodiimide metho-p-toluenesulfonate, or 1,3-dicyclohexylcarbodiimide (DCC). In some embodiments, the coupling reagent is N-(3-dimethylaminopropyl)-N-ethylcarbodiimide (EDC or EDCI), N-(3-dimethylaminopropyl)-N-ethylcarbodiimide hydrochloride (EDC hydrochloride), 1-[3-(dimethylamino)propyl]-3-ethylcarbodiimide methiodide (EDC methiodide), 1-cyclohexyl-3-(2-morpholinoethyl)carbodiimide metho-p-toluenesulfonate, or 1,3-dicyclohexylcarbodiimide (DCC). In some embodiments, the coupling reagent present in the reacting of the compound of Formula F6 with the carboxylic acid of Formula F7 is N-(3-dimethylaminopropyl)-N-ethylcarbodiimide (EDC or EDCI). In some embodiments, the coupling reagent present in the reacting of the compound of Formula F6 with the carboxylic acid of Formula F7 is N-(3-dimethylaminopropyl)-N-ethylcarbodiimide hydrochloride (EDC hydrochloride).

In some embodiments, the base is a catalytic base. In some embodiments, the molar ratio of catalytic base to the compound of Formula F6-CSA is about 0.6:1.0, about 0.5:1.0, about 0.4:1.0, about 0.3:1.0, about 0.27:1.0, or about 0.25:1.0. In some embodiments, the catalytic base present in the reacting of the compound of Formula F6 with the carboxylic acid of Formula F7 is an organic base. In some embodiments, the catalytic base is an inorganic base. In some embodiments, the catalytic base is an organic base. In some embodiments, the catalytic base is sodium hydrogen carbonate, sodium carbonate, sodium citrate, sodium hydroxide, potassium hydroxide, or 4-dimethylaminopyridine. In some embodiments, the catalytic base is sodium hydroxide. In some embodiments, the catalytic base is potassium hydroxide. In some embodiments, the catalytic base present in the reacting of the compound of Formula F6 with the carboxylic acid of Formula F7 is dimethylaminopyridine (DMAP).

In some embodiments, the reacting of the compound of Formula F6 with the carboxylic acid of Formula F7 is conducted at a temperature below about 25° C. In some embodiments, the reacting of the compound of Formula F6 with the carboxylic acid of Formula F7 is conducted at a temperature ranging from about −10° C. to about 25° C. In some embodiments, the reacting of the compound of Formula F6 with the carboxylic acid of Formula F7 is conducted at a temperature ranging from about −5° C. to about 20° C. In some embodiments, the reacting of the compound of Formula F6 with the carboxylic acid of Formula F7 is conducted at a temperature ranging from about −5° C. to about 15° C. In some embodiments, the reacting of the compound of Formula F6 with the carboxylic acid of Formula F7 is conducted at a temperature ranging from about −5° C. to about 10° C. In some embodiments, the reacting of the compound of Formula F6 with the carboxylic acid of Formula F7 is conducted at a temperature ranging from about −1° C. to about 25° C. In some embodiments, the reacting of the compound of Formula F6 with the carboxylic acid of Formula F7 is conducted at a temperature ranging from −1° C. to 25° C.

In some embodiments, the process further comprises crystallizing the material comprising the compound of Formula I, comprising:

a) dissolving the material comprising the compound of Formula I in a solvent mixture comprising an alcohol and acetonitrile; and

b) crystallizing the compound of Formula I from the solvent mixture to afford a compound of

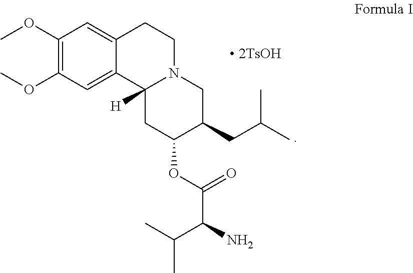 [SMILES: COc1cc2c(cc1OC)[C@H]1C[C@@H](OC(=O)[C@@H](N)C(C)C)[C@H](CC(C)C)CN1CC2]

In some embodiments, the process further comprises the step of crystallizing the compound of Formula I, comprising:

a) dissolving the compound of Formula I in a solvent mixture comprising an alcohol (e.g., methanol), and acetonitrile; and

b) crystallizing the compound of Formula I to afford a crystalline form of the compound of Formula I.

In some embodiments, the volume ratio of alcohol to acetonitrile in the solvent mixture is about 1:1 to about 1:3.5. In some embodiments, the volume ratio of alcohol to acetonitrile in the solvent mixture is about 1:1.5 to about 1:3. In some embodiments, the volume ratio of alcohol to acetonitrile in the solvent mixture is about 1:1.7 to about 1:2.7. In some embodiments, the volume ratio of alcohol to acetonitrile in the solvent mixture is about 1:1.8 to about 1:2.2. In some embodiments, the volume ratio of alcohol to acetonitrile in the solvent mixture is about 1:1.9 to about 1:2.1. In some embodiments, the volume ratio of alcohol to acetonitrile in the solvent mixture is about 1:2.

In some embodiments, the ratio of alcohol to acetonitrile in the solvent mixture is approximately 1:2 v/v. In some embodiments, the alcohol is ethanol or methanol. In some embodiments, the alcohol is methanol.

In some embodiments, the crystallizing in step b) comprises seeding the resulting solvent and compound mixture with a crystal of the compound of Formula I to form a seed mixture. In some embodiments, the crystallizing in step b) comprises seeding the resulting solvent and compound mixture with a crystal of the compound of Formula I to form a seed mixture and cooling the seeded mixture. In some embodiments, the crystallizing in step b) comprises removing from about 10% to about 99% of the alcohol (e.g., methanol) by weight or volume of the alcohol (e.g., methanol), based on an initial amount of alcohol (e.g., methanol). In some embodiments, the seed mixture is heated to a temperature of about 30° C. to about 50° C. prior to and/or during seeding. In some embodiments, the seed mixture is heated to a temperature of about 37° C. to about 47° C. prior to and/or during seeding. In some embodiments, the seed mixture is heated to a temperature of about 39° C. to about 45° C. prior to and/or during seeding. In some embodiments, the seed mixture is heated to a temperature of about 41° C. to about 43° C. prior to and/or during seeding. In some embodiments, the seed mixture is heated to a temperature of about 42° C. prior to and/or during seeding. In some embodiments, after heating the seed mixture, the resulting seed mixture is cooled to a temperature of about 15° C. to about 25° C. In some embodiments, after heating the seed mixture, the resulting seed mixture is cooled to a temperature of about 16° C. to about 24° C. In some embodiments, after heating the seed mixture, the resulting seed mixture is cooled to a temperature of about 17° C. to about 23° C. In some embodiments, after heating the seed mixture, the resulting seed mixture is cooled to a temperature of about 18° C. to about 22° C. In some embodiments, after heating the seed mixture, the resulting seed mixture is cooled to a temperature of about 19° C. to about 21° C. In some embodiments, after heating the seed mixture, the resulting seed mixture is cooled to a temperature of about 20° C.

In some embodiments, the crystalline form of the compound of Formula I is isolated and drying at elevated temperature under vacuum. In some embodiments, the crystalline form of the compound of Formula I is Form I, as described herein.

In some embodiments, the crystallizing in Step b) of crystallizing the material comprising the compound of Formula I comprises seeding the resulting solvent and compound mixture with a crystal of a compound of Formula I and cooling the seeded mixture. In some embodiments, the solvent and compound mixture are heated to between about 30° C. and about 50° C. prior to or during seeding. In some embodiments, the solvent and compound mixture is cooled to between approximately 15° C. and approximately 25° C. immediately after heating.

In some embodiments, the crystallizing in step b) of crystallizing the material comprising the compound of Formula I comprises removing from 5%, 10%, 12%, 14%, 16%, 18%, 20%, 25%, 30%, 35%, 40%, 45%, 50%, 55%, 60%, 65%, 70%, 75%, 80%, 85%, 90%, 95%, 99%, 99.5% by weight or volume of the alcohol, based on an initial amount, or an amount within a range defined by any of the preceding amounts. In some embodiments, the crystallizing in step b) of crystallizing the material comprising the compound of Formula I comprises removing from about 10% to about 99% by weight or volume of the alcohol, based on an initial amount of alcohol. In some embodiments, the alcohol is a CrC₆ alcohol. In some embodiments, the alcohol is ethanol or methanol. In some embodiments, the alcohol is methanol.

In some embodiments, the compound of Formula I is dried under vacuum at elevated temperature. In some embodiments, the compound of Formula I is dried under vacuum at about 50° C. for no less than 12 hours.

In some embodiments the compound of Formula I has a purity of no less than about 95% by weight, no less than about 96% by weight, no less than about 97% by weight, no less than about 97.5% by weight, no less than about 98% by weight, no less than about 98.5% by weight, no less than about 99% by weight, no less than about 99.10% by weight, no less than about 99.2% by weight, no less than about 99.3% by weight, no less than about 99.4% by weight, no less than about 99.5% by weight, no less than about 99.6% by weight, no less than about 99.7% by weight, no less than about 99.8% by weight, or no less than about 99.9% by weight. In some embodiments, the compound of Formula F6-CSA has a purity of at least 99.5%.

In some embodiments, the compound of Formula F6 is prepared by a process comprising reacting a compound of Formula F6-CSA:

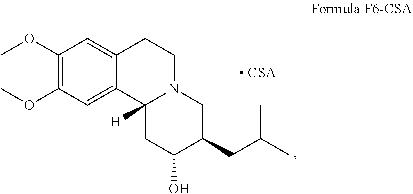 [SMILES: COc1cc2c(cc1OC)[C@H]1C[C@@H](O)[C@H](CC(C)C)CN1CC2]

with a base to afford the compound of Formula F6.

In some embodiments, the base which is reacted with the compound of Formula F6-CSA is an inorganic base. In some embodiments, the base is sodium hydrogen carbonate, sodium carbonate, sodium citrate, sodium hydroxide, or potassium hydroxide. In some embodiments, the base is sodium hydroxide.

In some embodiments, the base is potassium hydroxide. In some embodiments, the reacting of the compound of Formula 6-CSA with a base is performed in a solvent comprising a hydrocarbon, chlorinated hydrocarbon, alcohol, ether, ester, carbonate, amide, nitrile, sulfoxide, sulfone, nitro compound, heteroarene, heterocycle, water, or a mixture thereof. In some embodiments, the solvent is a chlorinated hydrocarbon solvent. In some embodiments, the solvent is dichloromethane. In some embodiments, the solvent is an ether. In some embodiments the solvent is a cycloalkyl ether. In some embodiments, the solvent is 2-methyltetrahydroforan (MeTHF). In some embodiments, the solvent comprises water and a halogenated hydrocarbon solvent. In some embodiments, the halogenated hydrocarbon solvent is dichloromethane.

In some embodiments, the reacting of the compound of Formula 6-CSA with a base is performed at a temperature of about 20° C. to about 30° C. In some embodiments, the reacting of the compound of Formula 6-CSA with a base is performed at a temperature of about 21° C. to about 29° C. In some embodiments, the reacting of the compound of Formula 6-CSA with a base is performed at a temperature of about 22° C. to about 28° C. In some embodiments, the reacting of the compound of Formula 6-CSA with a base is performed at a temperature of about 23° C. to about 27° C. In some embodiments, the reacting of the compound of Formula 6-CSA with a base is performed at a temperature of about 24° C. to about 26° C. In some embodiments, the reacting of the compound of Formula 6-CSA with a base is performed at a temperature of about 25° C.

In some embodiments, the compound of Formula F6-CSA is prepared by the process comprising reacting a compound of Formula F5:

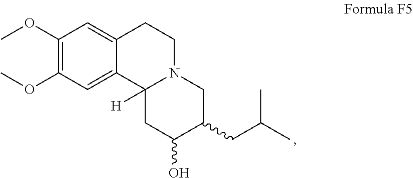 [SMILES: COc1cc2c(cc1OC)C1CC(O)C(CC(C)C)CN1CC2]

with (S)-(+)-camphorsulfonic acid (CSA) to afford the compound of Formula F6-CSA.

In some embodiments, the molar ratio of CSA to the compound of Formula F5 is about 0.7:1 to about 1:1. In some embodiments, the molar ratio of CSA to the compound of Formula F5 is about 0.75:1 to about 0.95:1. In some embodiments, the molar ratio of CSA to the compound of Formula F5 is about 0.7:1 to about 0.9:1. In some embodiments, the molar ratio of CSA to the compound of Formula F5 is about 0.8:1 to about 0.9:1. In some embodiments, the molar ratio of CSA to the compound of Formula F5 is about 0.8:1 to about 0.85:1. In some embodiments, the molar ratio of CSA to the compound of Formula F5 is about 0.8:1. In some embodiments, the molar ratio of CSA to the compound of Formula F5 is 0.825:1.

In some embodiments, the molar ratio of CSA to the compound of Formula F5 is about 0.66:1 to about 0.99:1. In some embodiments, the molar ratio of CSA to the compound of Formula F5 is about 0.70:1 to about 0.95:1. In some embodiments, the molar ratio of CSA to the compound of Formula F5 is about 0.74:1 to about 0.91:1. In some embodiments, the molar ratio of CSA to the compound of Formula F5 is about 0.76:1 to about 0.89:1. In some embodiments, the molar ratio of CSA to the compound of Formula F5 is about 0.78:1 to about 0.87:1. In some embodiments, the molar ratio of CSA to the compound of Formula F5 is about 0.80:1 to about 0.85:1. In some embodiments, the molar ratio of CSA to the compound of Formula F5 is about 0.81:1 to about 0.84:1.

In some embodiments, the reacting of the compound of Formula F5 is performed in a solvent comprising water and an alcohol. In some embodiments, the alcohol is a C₁-C₆ alcohol. In some embodiments, the solvent is a solvent mixture. In some embodiments, the solvent mixture comprises water and ethanol. In some embodiments, the solvent mixture comprises water and ethanol in a volume ratio of water to ethanol of about 1:5 to about 1:25. In some embodiments, the solvent mixture comprises water and ethanol in a volume ratio of water to ethanol of about 1:10 to about 1:20. In some embodiments, the solvent mixture comprises water and ethanol in a volume ratio of water to ethanol of about 1:14 to about 1:18. In some embodiments, the solvent mixture comprises water and ethanol in a volume ratio of water to ethanol of about 1:15 to about 1:17. In some embodiments, the solvent mixture comprises water and ethanol in a volume ratio of water to ethanol of about 1:15.5 to about 1:16.5. In some embodiments, the solvent mixture comprises water and ethanol in a volume ratio of water to ethanol of about 1:16. In some embodiments, the solvent mixture comprises water and ethanol in a volume ratio ranging from about 0.1 to about 100, from about 0.2 to about 50, from about 0.5 to about 25, from about 1 to about 20, from about 1 to about 16, from about 1 to about 10, from about 1 to about 5, or from about 1 to about 2. In some embodiments, the solvent comprising ethanol and water comprises about 10-14 volumes of ethanol and about 0.5-1.0 volumes of water. In some embodiments, the solvent comprising ethanol and water comprises about 12 volumes of ethanol and about 0.75 volumes of water.

In some embodiments, the reacting of the compound of Formula F5 takes place at a temperature ranging from about 20 to about 80° C., from about 20 to about 70° C., from about 20 to about 60° C., from about 20 to about 70° C. In other embodiments, the reaction is conducted at temperature ranging from about 20 to about 65° C., or from about 20 to about 75° C. In some embodiments, the reacting of the compound of Formula F5 takes place at a temperature of about 70° C. In some embodiments, the reacting of the compound is cooled from about 70° C. to about 55° C. and allowed to crystallize. In some embodiments, the reaction mixture of Formula F5 and CSA is cooled to about 22° C. In some embodiments, the reaction mixture is seeded with a crystal of a compound of Formula F6-CSA. In some embodiments, the compound of Formula F6-CSA is dried under vacuum at elevated temperature. In some embodiments, the compound of Formula F6-CSA is dried under vacuum at about 45° C. for no less than 12 hours.

In some embodiments, the compound of Formula F6-CSA has an optical purity of no less than about 95%, no less than about 96%, no less than about 97%, no less than about 97.5%, no less than about 98%, no less than about 98.5%, no less than about 99%, no less than about 99.1%, no less than about 99.2%, no less than about 99.3%, no less than about 99.4′%, no less than about 99.5%, no less than about 99.6%, no less than about 99.7%, no less than about 99.8%, or no less than about 99.9%. In some embodiments, the compound of Formula F6-CSA has an optical purity greater than 99%.

In some embodiments, the compound of Formula F5 is prepared by the process comprising reacting a compound of Formula F4:

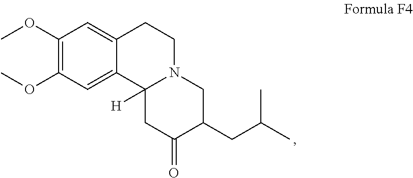 [SMILES: COc1cc2c(cc1OC)C1CC(=O)C(CC(C)C)CN1CC2]

with a reducing agent in a solvent comprising methyl tert-butyl ether (MTBE) and methanol to afford the compound of Formula F5.

In some embodiments, the solvent comprises MTBE and an alcohol that is not methanol. In some embodiments, the alcohol is a C₂-C₆ alcohol (containing 2 to 6 carbon atoms). In yet other embodiments, the solvent mixture comprises MTBE and ethanol. In some embodiments, the reacting of a compound of Formula F4 is conducted in the presence of an organic acid. In some embodiments, the organic acid is a carboxylic acid. In some embodiments, the organic acid is a C₁₋₄ carboxylic acid (containing 1 to 14 carbon atoms) optionally substituted with one or more substituents Q. In some embodiments, the acid is a 2-hydroxy-C₁₋₁₄ carboxylic acid (containing 1 to 14 carbon atoms), optionally substituted with one or more substituents Q. The one or more substituents Q, are each independently selected from, e.g., (a) oxo (O═O), halo, cyano (—CN), and nitro (—NO₂); (b) C₁₋₆alkyl, C₂₋₆ alkenyl, C₂₋₆alkynyl, C₃₋₁₀cycloalkyl, C₆₋₁₄ aryl, C₁₋₅ aralkyl, heteroaryl, and heterocyclyl, each of which is further optionally substituted with one or more, in one embodiment, one, two, three, or four, substituents Q^{a}; and (c)—C(O)R^{a}, —C(O)OR^{a}, —C(O)NR^{b}R^{b}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)R^{d}, —NR^{a}(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂N^{b}R, —P(O)R^{a}R^{d}, —P(O)(OR^{a})R^{d},-P(O)(OR^{a})(OR^{d}), —SR^{a}—S(O)R^{a}, —S(O)₂R^{a}, —S(O)NR^{b}R^{c}, and—S(O)₂NR^{b}R^{c}, wherein each R^{a}, R^{b}, R^{c} and R^{d} is independently (i) hydrogen; (ii) C₁₋₆ alkyl, C₂₋₆ alkenyl, C₂₋₆ alkynyl, C₃₋₁₀ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R and R together with the N atom to which they are attached form heteroaryl or heterocyclyl, each of which optionally substituted with one or more, in one embodiment, one, two, three, or four, substituents Q^{a}.

In some embodiments, the acid is acetic acid, formic acid, oxalic acid, maleic acid, lactic acid, ascorbic acid, mandelic acid, or a mixture thereof. In some embodiments, the organic acid is acetic acid.

In some embodiments, the volume ratio of MTBE and methanol is ranging from about 1:1 to about 10:1. In some embodiments, the volume ratio of MTBE and methanol is ranging from about 1:1 to about 5:1. In some embodiments, the volume ratio of MTBE and methanol is ranging from about 3:1 to about 7:1. In some embodiments, the volume ratio of MTBE and methanol is ranging from about 3:1 to about 5:1. In some embodiments, the volume ratio of MTBE and methanol is about 4.4:1.

In some embodiments, the solvent comprising methyl tert-butyl ether (MTBE) and methanol further comprises an acid. In some embodiments, the acid comprises acetic acid. In some embodiments, the acid is acetic acid.

In some embodiments, the acid is present in excess compared to the compound of Formula F4. In some embodiments, the acid comprises acetic acid. In some embodiments, the acetic acid is present in about 0.5 to about 1.5 equivalents to the compound of Formula F4. In some embodiments, the acid comprises acetic acid. In some embodiments, the acetic acid is present in about 0.8 to about 1.3 equivalents to the compound of Formula F4. In some embodiments, the acid comprises acetic acid. In some embodiments, the acetic acid is present in about 0.9 to about 1.2 equivalents to the compound of Formula F4. In some embodiments, the acid comprises acetic acid. In some embodiments, the acetic acid is present in about 1.0 to about 1.2 equivalents to the compound of Formula F4. In some embodiments, the acetic acid is present in about 0.7 to about 1.0 equivalents to the compound of Formula F4. In some embodiments, the acetic acid is present in about 0.9 equivalents to the compound of Formula F4. In some embodiments, the acetic acid is present in about 0.8 equivalents to the compound of Formula F4.

In some embodiments, the reducing agent is initially added to the compound of Formula F4 as a slurry in MTBE. In some embodiments, the reducing agent is initially added to the compound of Formula F4 as a solid. In some embodiments, the reducing agent is a borohydride reducing agent. In some embodiments, the reducing agent is a borohydride. In some embodiments, the reducing agent is sodium borohydride, lithium borohydride, calcium borohydride, magnesium borohydride, potassium borohydride, 9-BBN, cyano borohydride, bis-triphenylphosphine borohydride, sodium triethyl borohydride, tetrabutylammonium borohydride, tetramethylammonium borohydride, tetraethylammonium borohydride, or lithium triethyl borohydride. In some embodiments, the borohydride reducing agent is sodium borohydride. In some embodiments, the reducing agent is sodium borohydride and is initially added as a solid. In some embodiments, the molar ratio of sodium borohydride to the compound of Formula F4 is ranging from about 1.0 to about 10.0. In some embodiments, the molar ratio of sodium borohydride to the compound of Formula F4 is ranging from about 1.0 to about 5.0. In some embodiments, the molar ratio of sodium borohydride to the compound of Formula F4 is ranging from about 1.0 to about 3.0. In some embodiments, the molar ratio of sodium borohydride to the compound of Formula F4 is ranging from about 1.5 to about 2.5. In some embodiments, the molar ratio of sodium borohydride to the compound of Formula F4 is ranging from about 1.8 to about 2.2. In some embodiments, the molar ratio of sodium borohydride to the compound of Formula F4 is ranging from about 1.9 to about 2.1. In some embodiments, the molar ratio of sodium borohydride to the compound of Formula F4 is about 2.0.

In some embodiments, lithium chloride is not present in the reacting of a compound of Formula F4.

In some embodiments, the reacting of the compound of Formula F4 with a reducing agent is conducted at a temperature ranging from about minus 5° C. to about minus 15° C., from about minus 5° C. to about minus 10° C., from about minus 5° C. to about 0° C., from about 0° C. to about 5° C., from about 0 to about 10° C., from about 0° C. to about 15° C., from about 0° C. to about 25° C., from about 0° C. to about 30° C., from about 5° C. to about 30° C., from about 10° C. to about 30° C., from about 20° C. to about 30° C., from about 20° C. to about 25° C., from about 20° C. to about 24° C., and from about 21° C. to about 23°±C.

In some embodiments, the reacting of the compound of Formula F4 with a reducing agent is conducted at a temperature of about 15° C. to about 30° C. In some embodiments, the reacting of the compound of Formula F4 with a reducing agent is conducted at a temperature of about 20° C. to about 27°±C. In some embodiments, the reacting of the compound of Formula F4 with a reducing agent is conducted at a temperature of about 21° C. to about 26° C. In some embodiments, the reacting of the compound of Formula F4 with a reducing agent is conducted at a temperature of about 22° C. to about 25°±C.

In some embodiments, the reacting of the compound of Formula F4 with a reducing agent is conducted at a temperature of about 25° C. In some embodiments, the reacting of the compound of Formula F8 with a reducing agent is carried out over a period of about 2 hours. In some embodiments, the reacting of the compound of Formula F8 with a reducing agent is carried at a temperature ranging from about 15° C. to about 30° C. and over a period of at least 1.5 hours. In some embodiments, the reacting of the compound of Formula F8 with a reducing agent is carried at a temperature ranging from about 15° C. to about 30° C. and over a period of about 1 hours to about 3 hours. In some embodiments, the reacting of the compound of Formula F8 with a reducing agent is carried at a temperature ranging from about 18° C. to about 28° C. and over a period of about 1.5 hours to about 2.5 hours. In some embodiments, the reacting of the compound of Formula F8 with a reducing agent is carried at a temperature ranging from about 20° C. to about 28° C. and over a period of about 1.8 hours to about 2.2 hours.

In some embodiments, the compound of Formula F4 is prepared by the process comprising reacting a compound of Formula F3:

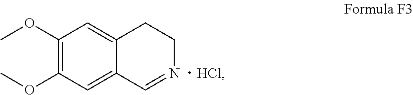

with a compound of Formula F2:

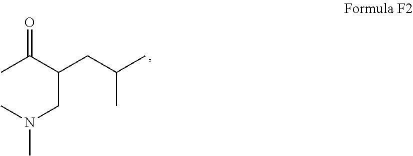

in a solvent comprising isopropanol (IPA) and water to afford the compound of Formula F4.

In some embodiments, the volume ratio of IPA and water is ranging from about 1:1 to about 10:1. In some embodiments, the volume ratio of IPA and water is ranging from about 1:1 to about 5:1. In some embodiments, the volume ratio of IPA and water is ranging from about 1:1 to about 3:1. In some embodiments, the volume ratio of IPA and water is ranging from about 2:1 to about 3:1. In some embodiments, the volume ratio of IPA and water is about 2:1 to about 2.6:1. In some embodiments, the volume ratio of IPA and water is about 2.1:1 to about 2.5:1. In some embodiments, the volume ratio of IPA and water is about 2.2:1 to about 2.4:1. In some embodiments, the volume ratio of IPA and water is about 2.25:1 to about 2.35:1. In some embodiments, the volume ratio of IPA and water is about 2.3:1.

In some embodiments, the reacting of the compound of Formula F3 with the compound of Formula F2 is in a solvent that is not IPA and water. In some embodiments, the solvent is a hydrocarbon, chlorinated hydrocarbon, alcohol, ether, ketone, ester, carbonate, amide, nitrile, sulfoxide, sulfone, nitro compound, heteroarene, heterocycle, carboxylic acid, phosphoramide, carbon sulfide, water, or a mixture thereof. In some embodiments, the solvent is petroleum ether, pentane, hexane(s), heptane, octane, isooctane, cyclopentane, cyclohexane, methylcyclohexane, benzene, toluene, xylene, tetralin, cumene, dichloromethane (DCM), 1,2-dichloroethane, 1,1-dichloroethene, 1,2-dichloroethene, chloroform, trichloroethane, trichloroethene, carbon tetrachloride, chlorobenzene, trifluoromethylbenzene, methanol, ethanol, isopropanol (IPA), 1-propanol, 1-butanol, 2-butanol, t-butanol, 3-methyl-1-butanol, 1-pentanol, 2-methoxyethanol, 2-ethoxyethanol, ethyleneglycol, diethyl ether, diisopropyl ether, methyl t-butyl ether (MTBE), diphenyl ether, 1,2-dimethoxyethane, bi(2-methoxyethyl)ether, 1,1-dimethoxymethane, 2,2-dimethoxypropane, anisole, acetone, butanone, methyl ethyl ketone (MEK), methyl isopropyl ketone, methyl butyl ketone, methyl isobutyl ketone (MIBK), methyl acetate, ethyl formate, ethyl acetate, propyl acetate, isopropyl acetate, isobutyl acetate, butyl acetate, ethylene carbonate, propylene carbonate, formamide, N,N-dimethylformamide (DMF), N,N-dimethylacetamide, acetonitrile (ACN), dimethyl sulfoxide (DMSO), sulfolane, nitromethane, nitrobenzene, N-methyl pyrrolidone, 2-methyl tetrahydrofuran, tetrahydrofuran (THF), dioxane, pyridine, formic acid, acetic acid, trichloroacetic acid, trifluoroacetic acid, hexamethylphosphoramide, carbon sulfide, water; or a mixture thereof.

In some embodiments, the reacting of the compound of Formula F3 with the compound of Formula F2 takes place in the presence of sodium iodide. In some embodiments, the molar ratio of sodium iodide to the compound of Formula F3 is ranging from about 0.1:1 to 1:1. In some embodiments, the molar ratio of sodium iodide to the compound of Formula F3 is ranging from about 0.1:1 to 0.5:1. In some embodiments, the molar ratio of sodium iodide to the compound of Formula F3 is about 0.2:1 to 0.8:1. In some embodiments, the molar ratio of sodium iodide to the compound of Formula F3 is about 0.2:1 to 0.6:1. In some embodiments, the molar ratio of sodium iodide to the compound of Formula F3 is about 0.25:1 to 0.55:1. In some embodiments, the molar ratio of sodium iodide to the compound of Formula F3 is about 0.3:1 to 0.5:1. In some embodiments, the molar ratio of sodium iodide to the compound of Formula F3 is about 0.35:1 to 0.45:1. In some embodiments, the molar ratio of sodium iodide to the compound of Formula F3 is about 0.4:1.

In some embodiments, the reacting of the compound of Formula F3 with the compound of Formula F2 is carried out at an elevated temperature. In some embodiments, the reacting of the compound of Formula F3 with the compound of Formula F2 is carried out at a temperature of about 20° C. to about 60° C. In some embodiments, the reacting of the compound of Formula F3 with the compound of Formula F2 is carried out at a temperature of about 25° C. to about 50° C. In some embodiments, the reacting of the compound of Formula F3 with the compound of Formula F2 is carried out at a temperature of about 30° C. to about 45° C. In some embodiments, the reacting of the compound of Formula F3 with the compound of Formula F2 is carried out at a temperature of about 35° C. to about 45° C. In some embodiments, the reacting of the compound of Formula F3 with the compound of Formula F2 is carried out at a temperature of about 36° C. to about 48° C. In some embodiments, the reacting of the compound of Formula F3 with the compound of Formula F2 is carried out at a temperature of about 39° C. to about 45° C. In some embodiments, the reacting of the compound of Formula F3 with the compound of Formula F2 is carried out at a temperature of about 41° C. to about 43°±C. In some embodiments, the reacting of the compound of Formula F3 with the compound of Formula F2 is carried out at a temperature of about 42° C.

In some embodiments, the reacting of the compound of Formula F3 with the compound of Formula F2 takes place for no less than about 24 hours. In some embodiments, the reacting of the compound of Formula F3 with the compound of Formula F2 takes place for about 24 hours.

In some embodiments, the compound of Formula F2 is prepared by the process comprising reacting a compound of Formula F1:

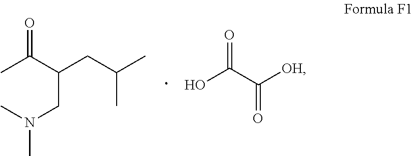

with a base to afford the compound of Formula F2.

In some embodiments, the base which is reacted with the compound of Formula I comprises an inorganic base. In some embodiments, the base is a carbonate, hydrogen carbonate or hydroxide base. In other embodiments, the base is sodium carbonate. In some embodiments, the base which is reacted with the compound of Formula F1 is potassium hydroxide.

In some embodiments, said reacting of the compound of Formula F1 and base is performed in a suitable solvent. In some embodiments, the suitable solvent is a mixture of solvents. In some embodiments, the mixture of solvents comprises water and an organic solvent. In some embodiments, the mixture of solvents comprises water and an ether solvent. In some embodiments, the organic solvent used in the reacting of the compound of Formula F1 is MTBE (i.e., methyl tert-butyl ether). In some embodiments, the mixture of solvents comprises water and MTBE. In some embodiments, said reacting of the compound of Formula F1 and base is performed in a solvent comprising water and an organic solvent. In some embodiments, the mixture of solvents comprises water and MTBE. In some embodiments, the volume ratio of water to MTBE is from about 1:1 to about 4:1. In some embodiments, the volume ratio of water to MTBE is from about 1.3:1 to about 3.5:1. In some embodiments, the volume ratio of water to MTBE is from about 1.8:1 to about 3:1. In some embodiments, the volume ratio of water to MTBE is from about 2.0:1 to about 2.8:1. In some embodiments, the volume ratio of water to MTBE is from about 2.3:1 to about 2.5:1. In some embodiments, the volume ratio of water to MTBE is from about 2.35:1 to about 2.45:1. In some embodiments, the volume ratio of water to MTBE is 2.4:1. In some embodiments, the solvent used in the reacting of the compound of Formula F1 is removed after completion of the reaction and replaced with isopropanol.

In some embodiments, the present application provides processes of preparing a compound of Formula I:

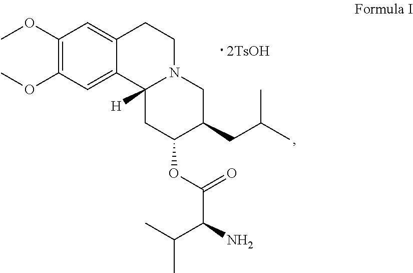 [SMILES: COc1cc2c(cc1OC)[C@H]1C[C@@H](OC(=O)[C@@H](N)C(C)C)[C@H](CC(C)C)CN1CC2]

comprising:

a) reacting a compound of Formula F1:

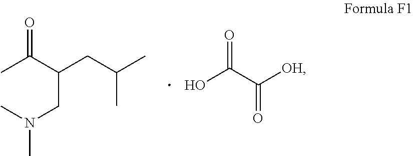

with a base, to afford a compound of Formula F2:

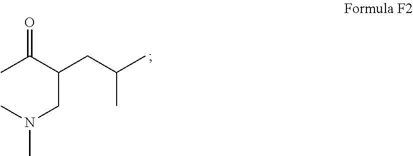

b) reacting a compound of Formula F3:

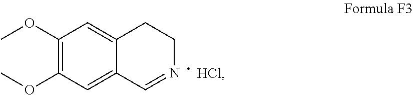

with a compound of Formula F2 in a solvent comprising isopropanol (IPA) and water to afford a compound of Formula F4:

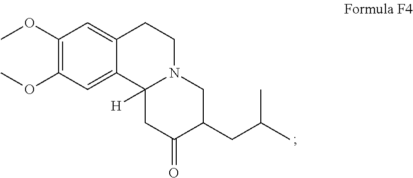 [SMILES: COc1cc2c(cc1OC)C1CC(=O)C(CC(C)C)CN1CC2]

c) reacting a compound of Formula F4 with a reducing agent in a solvent comprising methyl tert-butyl ether (MTBE) and methanol to afford a compound of Formula F5:

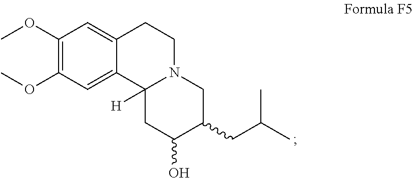 [SMILES: COc1cc2c(cc1OC)C1CC(O)C(CC(C)C)CN1CC2]

d) reacting a compound of Formula F5 with (S)-(+)-camphorsulfonic acid (CSA) to afford a compound of Formula F6-CSA:

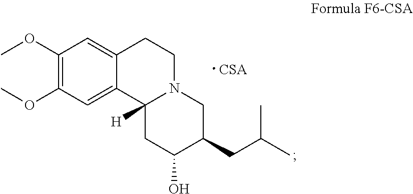

e) reacting a compound of Formula F6-CSA with a base to afford a compound of Formula F6:

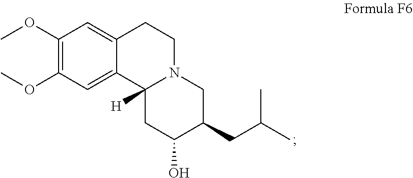 [SMILES: COc1cc2c(cc1OC)[C@H]1C[C@@H](O)[C@H](CC(C)C)CN1CC2]

f) reacting a compound of Formula F6 with a carboxylic acid of Formula T7:

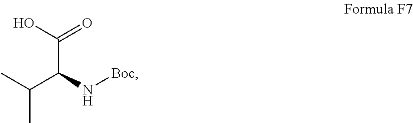

in a solvent to afford a product of Formula F8:

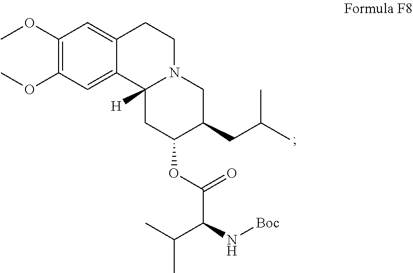 [SMILES: COc1cc2c(cc1OC)[C@H]1C[C@@H](OC(=O)[C@@H](NC(=O)OC(C)(C)C)C(C)C)[C@H](CC(C)C)CN1CC2]

g) reacting the product of Formula F8 with p-toluenesulfonic acid in acetonitrile or isopropyl acetate, to afford a material comprising the compound of Formula I; and

h) crystallizing the material comprising the compound of Formula I, comprising:
i) dissolving the material comprising the compound of Formula I in a solvent mixture comprising methanol and acetonitrile; and
ii) crystallizing the compound of Formula I from the solvent mixture to afford a compound of Formula I:

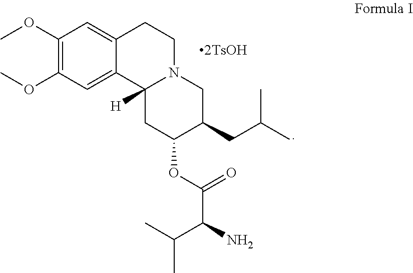 [SMILES: COc1cc2c(cc1OC)[C@H]1C[C@@H](OC(=O)[C@@H](N)C(C)C)[C@H](CC(C)C)CN1CC2]

In some embodiments, the present application provides a process of preparing a crystalline compound of Formula I, comprising:

a) dissolving a material comprising a compound of Formula I in a solvent mixture comprising methanol and acetonitrile; and

b) crystallizing the compound of Formula I from the solvent mixture to afford a crystalline compound of Formula I:

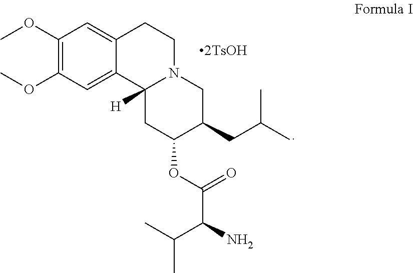 [SMILES: COc1cc2c(cc1OC)[C@H]1C[C@@H](OC(=O)[C@@H](N)C(C)C)[C@H](CC(C)C)CN1CC2]

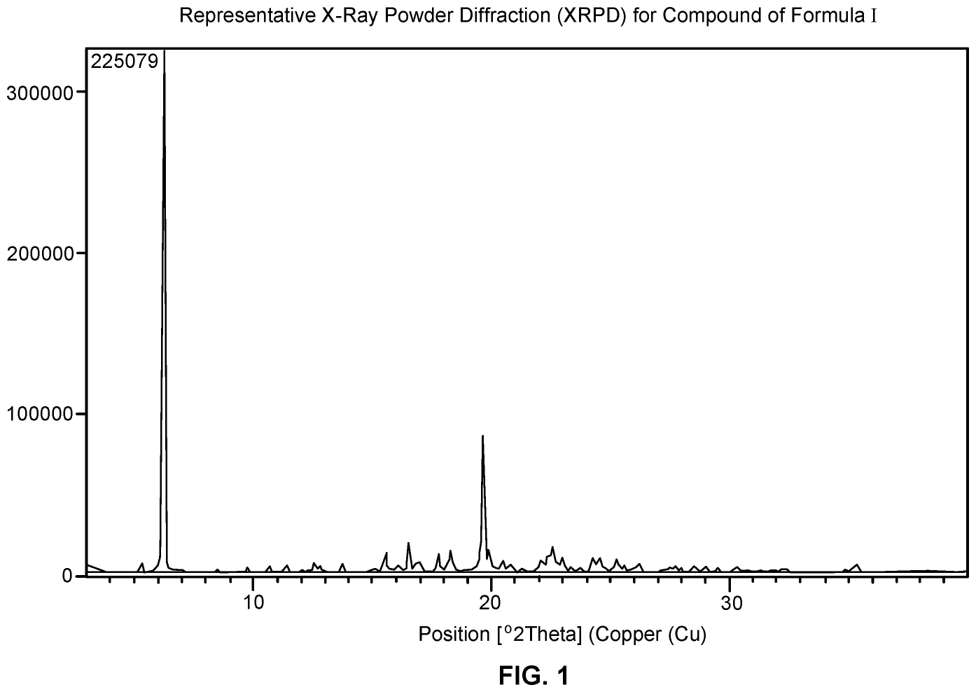

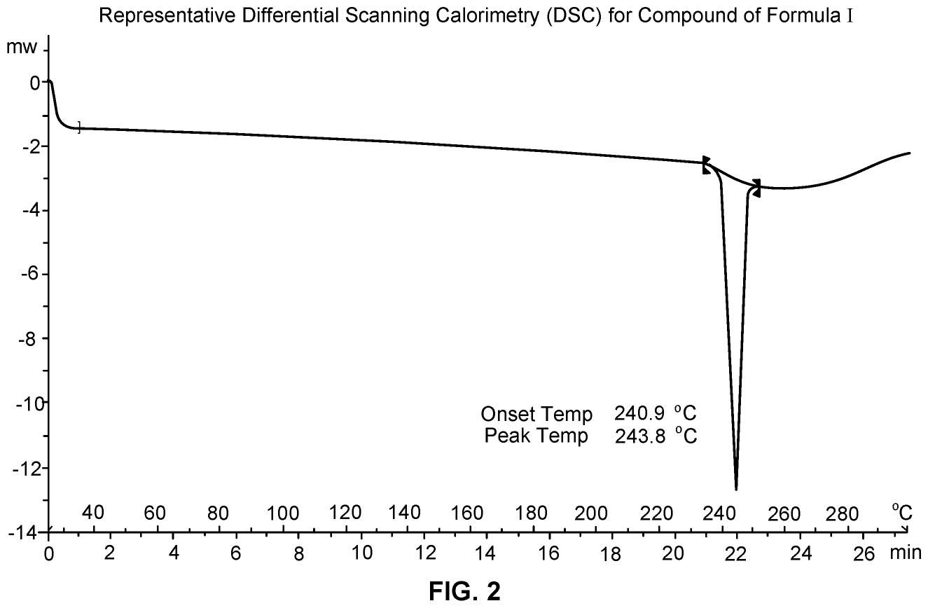

In some embodiments, the crystalline compound of Formula I is Form I as described herein, for example see Table 1, Table 2, Table 3, FIG. 1, and FIG. 2.

In some embodiments, the present application provides processes of preparing a compound of Formula I:

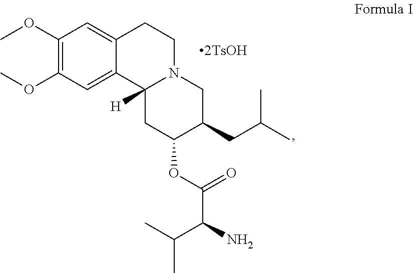 [SMILES: COc1cc2c(cc1OC)[C@H]1C[C@@H](OC(=O)[C@@H](N)C(C)C)[C@H](CC(C)C)CN1CC2]

comprising:

a) reacting a compound of Formula F6-CSA:

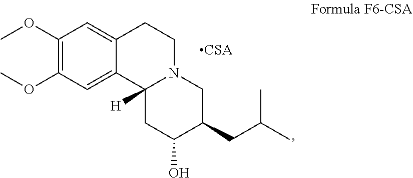 [SMILES: COc1cc2c(cc1OC)[C@H]1C[C@@H](O)[C@H](CC(C)C)CN1CC2]

with a base to afford a compound of Formula F6:

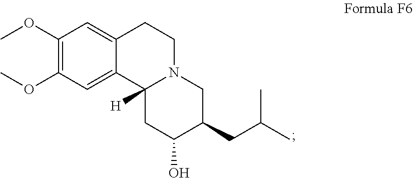 [SMILES: COc1cc2c(cc1OC)[C@H]1C[C@@H](O)[C@H](CC(C)C)CN1CC2]

b) reacting the compound of Formula F6 with a carboxylic acid of Formula F7:

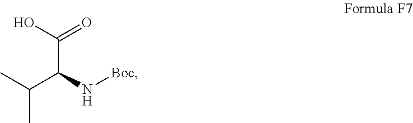

in a solvent to form a compound of Formula F8; and

c) reacting the compound of Formula F8:

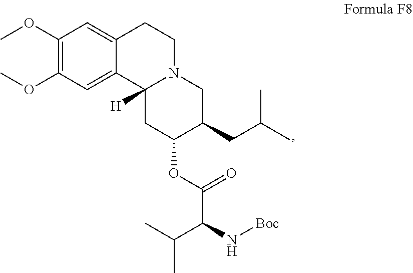 [SMILES: COc1cc2c(cc1OC)[C@H]1C[C@@H](OC(=O)[C@@H](NC(=O)OC(C)(C)C)C(C)C)[C@H](CC(C)C)CN1CC2]

with p-toluenesulfonic acid in a solvent comprising acetonitrile or isopropyl acetate, to afford a material comprising the compound of Formula I.

In some embodiments, the present application provides processes of preparing a compound of Formula F6-CSA:

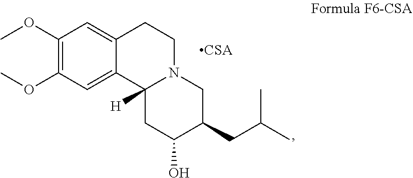 [SMILES: COc1cc2c(cc1OC)[C@H]1C[C@@H](O)[C@H](CC(C)C)CN1CC2]

comprising reacting a compound of Formula F5:

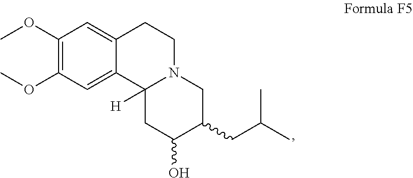 [SMILES: COc1cc2c(cc1OC)C1CC(O)C(CC(C)C)CN1CC2]

with (S)-(+)-camphorsulfonic acid (CSA), wherein the molar ratio of CSA to the compound of Formula F5 is between 0.7:1 and 0.9:1 to afford the compound of Formula F6-CSA.

In some embodiments, the present application provides processes of preparing a compound of Formula F5:

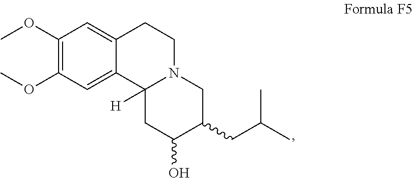 [SMILES: COc1cc2c(cc1OC)C1CC(O)C(CC(C)C)CN1CC2]

comprising reacting a compound of Formula F4:

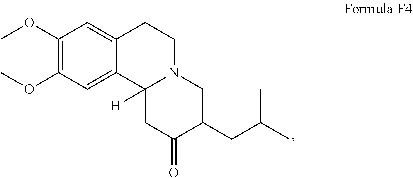 [SMILES: COc1cc2c(cc1OC)C1CC(=O)C(CC(C)C)CN1CC2]

with a reducing agent in a solvent comprising methyl tert-butyl ether (MTBE) and methanol to afford the compound of Formula F5.

In some embodiments, the present application provides processes of preparing a compound of Formula F4:

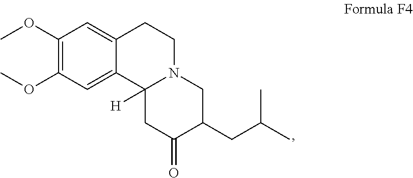 [SMILES: COc1cc2c(cc1OC)C1CC(=O)C(CC(C)C)CN1CC2]

comprising:

a) reacting a compound of Formula F1:

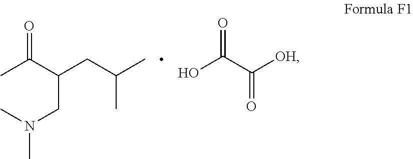

with a base to afford a compound of Formula F2:

and

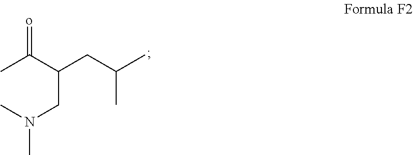

b) reacting the compound of Formula F2 with a compound of Formula F3:

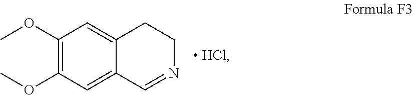

in the presence of sodium iodide in a solvent comprising isopropanol (IPA) and water to afford the compound of Formula F4.

In another embodiment, pharmaceutical compositions containing a compound of Formula I are disclosed. For the purposes of administration, the compound of Formula I may be formulated as a pharmaceutical composition. Pharmaceutical compositions disclosed herein comprise a compound of Formula I and a pharmaceutically acceptable carrier and/or diluent. The compound of Formula I is present in the composition in an amount which is effective to treat a particular disorder—that is, in an amount sufficient to reduce the supply of monoamines in the central nervous system, and preferably with acceptable toxicity to the patient. Appropriate concentrations and dosages can be readily determined by one skilled in the art.

Pharmaceutically acceptable carriers and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets which contain, in addition to a compound of Formula I, diluents, dispersing and surface-active agents, binders, and lubricants. One skilled in this art may further formulate the compound of Formula I in an appropriate manner, and in accordance with accepted practices, such as those disclosed in Remington's Pharmaceutical Sciences, Gennaro, Ed., Mack Publishing Co., Easton, Pa. 1990.

The pharmaceutical compositions may be formulated for systemic administration, which includes oral and parenteral methods of administration. For oral administration, suitable pharmaceutical compositions include powders, granules, pills, tablets, and capsules as well as liquids, syrups, suspensions, and emul-sions. These compositions may also include flavorants, preservatives, suspending, thickening and emulsifying agents, and other pharmaceutically acceptable additives. For parental administration, the compound of Formula I can be prepared in aqueous injection solutions which may con-tain, in addition to the compound of Formula I, buffers, antioxidants, bacteriostats, and other additives commonly employed in such solutions.

In some embodiments, the present application provides a process of for preparing a pharmaceutical composition comprising: preparing a compound of Formula I as provided herein and formulating the compound of Formula I with a pharmaceutically acceptable carrier and/or diluent.

In some embodiments, the compound of Formula I in the pharmaceutical composition is prepared by processes comprising:

a) reacting a compound of Formula F1:

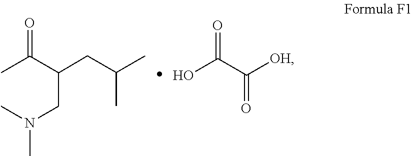

with a base, to afford a compound of Formula F2:

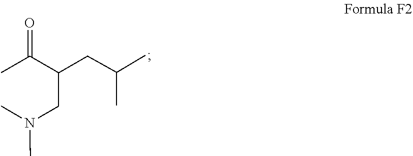

b) reacting a compound of Formula F3:

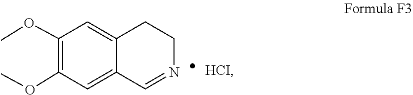

with a compound of Formula F2 in a solvent comprising isopropanol (IPA) and water to afford a compound of Formula F4:

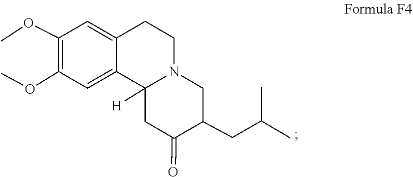 [SMILES: COc1cc2c(cc1OC)C1CC(=O)C(CC(C)C)CN1CC2]

c) reacting a compound of Formula F4 with a reducing agent in a solvent comprising methyl tert-butyl ether (MTBE) and methanol to afford a compound of Formula F5:

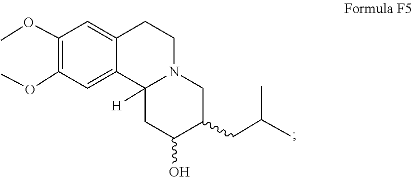 [SMILES: COc1cc2c(cc1OC)C1CC(O)C(CC(C)C)CN1CC2]

d) reacting a compound of Formula F5 with (S)-(+)-camphorsulfonic acid (CSA) to afford a compound of Formula F6-CSA:

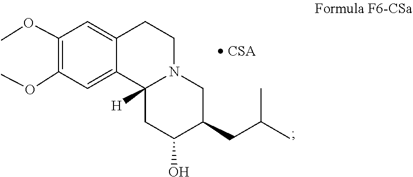 [SMILES: COc1cc2c(cc1OC)[C@H]1C[C@@H](O)[C@H](CC(C)C)CN1CC2]

e) reacting a compound of Formula F6-CSA with a base to afford a compound of Formula F6:

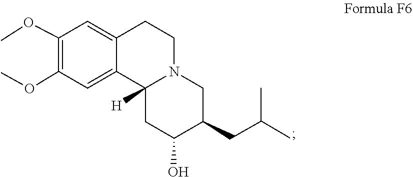 [SMILES: COc1cc2c(cc1OC)[C@H]1C[C@@H](O)[C@H](CC(C)C)CN1CC2]

f) reacting a compound of Formula F6 with a carboxylic acid of Formula F7:

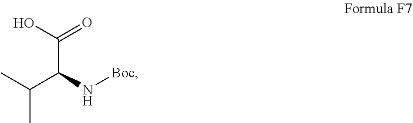

in a solvent to afford a product of Formula F8:

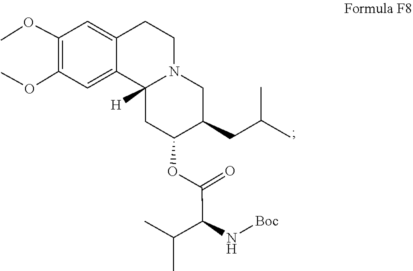 [SMILES: COc1cc2c(cc1OC)[C@H]1C[C@@H](OC(=O)[C@@H](NC(=O)OC(C)(C)C)C(C)C)[C@H](CC(C)C)CN1CC2]

g) reacting the product of Formula F8 with p-toluenesulfonic acid in acetonitrile or isopropyl acetate, to afford a material comprising the compound of Formula I; and

h) crystallizing the material comprising the compound of Formula I, comprising:
i) dissolving the material comprising the compound of Formula I in a solvent mixture comprising methanol and acetonitrile; and
ii) crystallizing the compound of Formula I from the solvent mixture to afford a compound of Formula I:

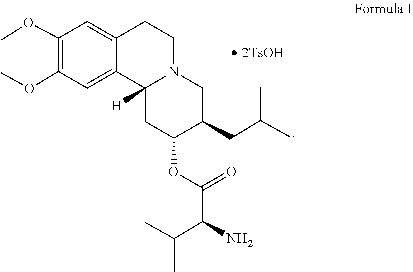 [SMILES: COc1cc2c(cc1OC)[C@H]1C[C@@H](OC(=O)[C@@H](N)C(C)C)[C@H](CC(C)C)CN1CC2]

In some embodiments, the compound of Formula I in the pharmaceutical composition is prepared by a process comprising reacting a compound of Formula F8:

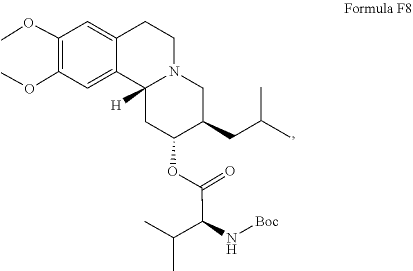 [SMILES: COc1cc2c(cc1OC)[C@H]1C[C@@H](OC(=O)[C@@H](NC(=O)OC(C)(C)C)C(C)C)[C@H](CC(C)C)CN1CC2]

with p-toluenesulfonic acid in a solvent comprising acetonitrile or isopropyl acetate, to afford a material comprising the compound of Formula I.

In some embodiments, the pharmaceutical composition comprises: the compound of Formula I (i.e., valbenazine ditosylate); at least one water insoluble filler; at least one water soluble diluent; at least one binder; at least one disintegrant; and at least one lubricant. In some embodiments, the pharmaceutical composition comprises: the compound of Formula I (i.e., valbenazine ditosylate) having a w/w % of about 40%; at least one water insoluble filler having a w/w % of about 25%; at least one water soluble diluent having a w/w % of about 20%; at least one binder having a w/w % of about 5%; at least one disintegrant having a w/w % of about 7.5%; and at least one lubricant having a w/w % of about 2.5%. In some embodiments, the pharmaceutical composition comprises: the compound of Formula I (i.e., valbenazine ditosylate) having a w/w % of about 40%; silicified microcrystalline cellulose having a w/w % of about 25%; isomalt having a w/w % of about 20%; hydroxypropyl methylcellulose having a w/w % of about 5%; partially pregelatinized maize starch having a w/w % of about 7.5%; and magnesium stearate having a w/w % of about 2.5%.

In some embodiments, the pharmaceutically acceptable carrier and/or diluent of the pharmaceutical composition comprises: silicified microcrystalline cellulose; isomalt; hydroxypropyl methylcellulose; partially pregelatinized maize starch; and magnesium stearate.

In some embodiments, the present application provides a compound of Formula I prepared by any of the processes as described herein, supra and infra. In some embodiments, the present application provides a compound of Formula I:

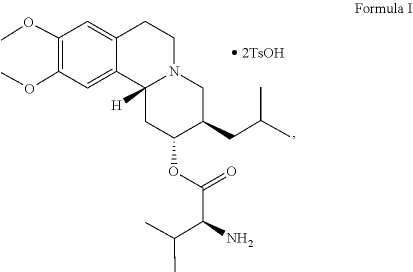 [SMILES: COc1cc2c(cc1OC)[C@H]1C[C@@H](OC(=O)[C@@H](N)C(C)C)[C@H](CC(C)C)CN1CC2]

prepared by a process comprising reacting a compound of Formula F8:

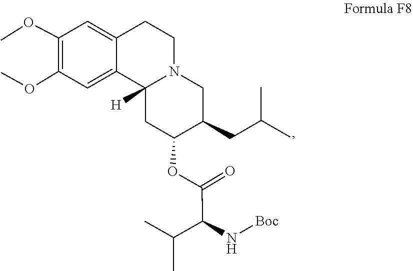 [SMILES: COc1cc2c(cc1OC)[C@H]1C[C@@H](OC(=O)[C@@H](NC(=O)OC(C)(C)C)C(C)C)[C@H](CC(C)C)CN1CC2]

with p-toluenesulfonic acid in a solvent comprising acetonitrile or isopropyl acetate, to afford a material comprising the compound of Formula I.

In some embodiments, the present application provides a compound of Formula I prepared by a process comprising:

a) reacting a product of Formula F8:

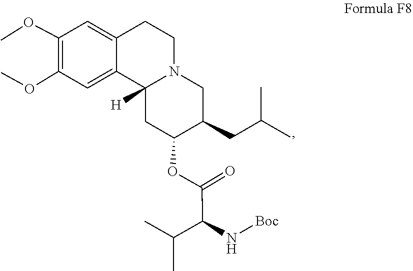 [SMILES: COc1cc2c(cc1OC)[C@H]1C[C@@H](OC(=O)[C@@H](NC(=O)OC(C)(C)C)C(C)C)[C@H](CC(C)C)CN1CC2]

with p-toluenesulfonic acid in acetonitrile or isopropyl acetate, to afford a material comprising the compound of Formula I; and

b) crystallizing the material comprising the compound of Formula I, comprising:
i) dissolving the material comprising the compound of Formula I in a solvent mixture comprising methanol and acetonitrile; and
ii) crystallizing the compound of Formula I from the solvent mixture to afford a compound of Formula I:

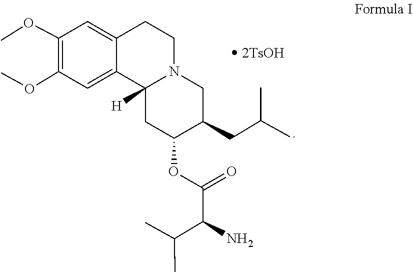 [SMILES: COc1cc2c(cc1OC)[C@H]1C[C@@H](OC(=O)[C@@H](N)C(C)C)[C@H](CC(C)C)CN1CC2]

In some embodiments, the present application provides a compound of Formula I prepared by a process comprising:

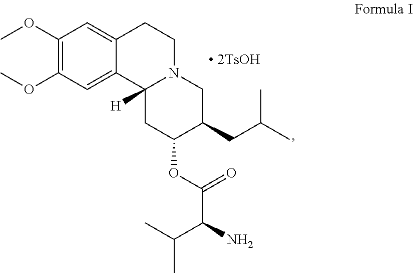 [SMILES: COc1cc2c(cc1OC)[C@H]1C[C@@H](OC(=O)[C@@H](N)C(C)C)[C@H](CC(C)C)CN1CC2]

a) reacting a compound of Formula F1:

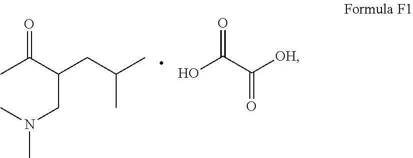

with a base, to afford a compound of Formula F2:

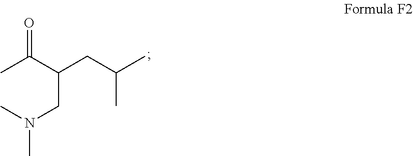

b) reacting a compound of Formula F3:

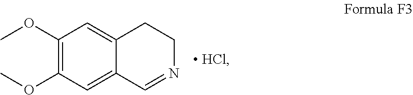

with a compound of Formula F2 in a solvent comprising isopropanol (IPA) and water to afford a compound of Formula F4:

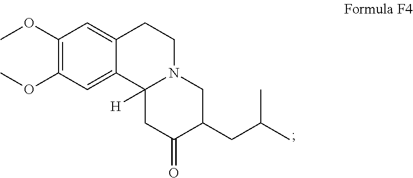 [SMILES: COc1cc2c(cc1OC)C1CC(=O)C(CC(C)C)CN1CC2]

c) reacting a compound of Formula F4 with a reducing agent in a solvent comprising methyl tert-butyl ether (MTBE) and methanol to afford a compound of Formula F5:

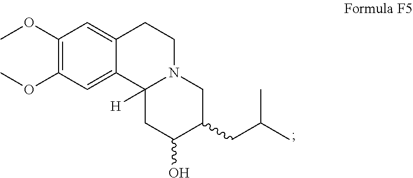 [SMILES: COc1cc2c(cc1OC)C1CC(O)C(CC(C)C)CN1CC2]

d) reacting a compound of Formula F5 with (S)-(+)-camphorsulfonic acid (CSA) to afford a compound of Formula F6-CSA:

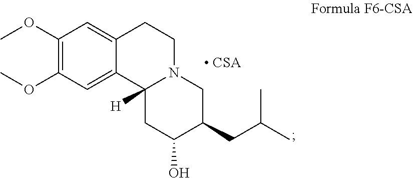 [SMILES: COc1cc2c(cc1OC)[C@H]1C[C@@H](O)[C@H](CC(C)C)CN1CC2]

e) reacting a compound of Formula F6-CSA with a base to afford a compound of Formula F6:

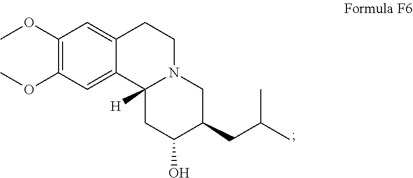 [SMILES: COc1cc2c(cc1OC)[C@H]1C[C@@H](O)[C@H](CC(C)C)CN1CC2]

f) reacting a compound of Formula F6 with a carboxylic acid of Formula F7:

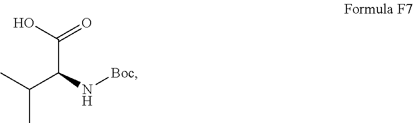

in a solvent to afford a product of Formula F8:

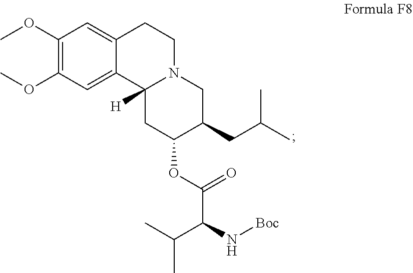 [SMILES: COc1cc2c(cc1OC)[C@H]1C[C@@H](OC(=O)[C@@H](NC(=O)OC(C)(C)C)C(C)C)[C@H](CC(C)C)CN1CC2]

g) reacting the product of Formula F8 with p-toluenesulfonic acid in acetonitrile or isopropyl acetate, to afford a material comprising the compound of Formula I; and

h) crystallizing the material comprising the compound of Formula I, comprising:
i) dissolving the material comprising the compound of Formula I in a solvent mixture comprising methanol and acetonitrile; and
ii) crystallizing the compound of Formula I from the solvent mixture to afford a compound of Formula I:

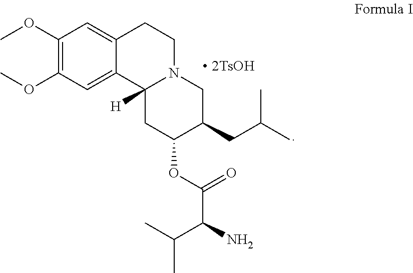 [SMILES: COc1cc2c(cc1OC)[C@H]1C[C@@H](OC(=O)[C@@H](N)C(C)C)[C@H](CC(C)C)CN1CC2]

Crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I).

In some embodiments, the present application provides a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I). The crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I) can be identified by the unique solid state signatures with respect to, for example, Differential Scanning Calorimetry (DSC), X-ray Powder Diffraction (XRPD), and other solid state methods. Further characterization with respect to water or solvent content of the crystalline forms can be gauged by any of the following methods for example, Thermogravimetric Analysis (TGA), DSC and the like.

For DSC, it is known that the temperatures observed for thermal events will depend upon sample purity and may also depend on the rate of temperature change, as well as sample preparation technique, and the instrument employed. Thus, the values reported herein relating to DSC thermograms can vary by plus or minus about 5° C. (i.e., ±about 5° C.). The values reported herein relating to DSC thermograms can also vary by plus or minus about 20 joules per gram (i.e., ±about 20 joules per gram).

For XRPD, the relative intensities of the peaks can vary, depending upon the sample preparation technique, the sample mounting procedure and the instrument employed. Moreover, instrument variation and other factors can often affect the 20 values. Therefore, the peak assignments of diffraction patterns can vary by plus or minus about 0.2°±(i.e., ±about 0.2°). For TGA, the temperature features reported herein can vary by plus or minus about 5° C. (i.e., ±about 5° C.). The TGA % weight changes reported herein over a specified temperature range can vary by plus or minus about 2% weight change (i.e., ±about 2% weight change) due to, for example, variations in sample quality and sample size. All X-ray powder diffraction patterns (diffractograms) were obtained using Cu-Kα radiation.

Further characterization with respect to hygroscopicity of the crystalline form can be gauged by, for example, Gravimetric Vapor Sorption (GVS). The GVS features can vary by plus or minus about 5% relative humidity (i.e., ±about 5% relative humidity). The GVS features can also vary by plus or minus about 2% weight change (i.e., ±about 2% weight change). One aspect of the present invention relates to a novel crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I) and processes related thereto.

A summary of representative physical properties for the crystalline form for the compound of Formula I are provided in Table 1, Table 2, and Table 3.

TABLE 1

| | (2R,3R,11bR)-3-isobutyl-9,10-dimethoxy-2,3,4,6,7,11b- |
|---|---|
| | hexahydro-1H-pyrido[2,l-a]isoquinolin-2-yl (S)-2-amino-3- |
| | methylbutanoate di(4-methylbenzenesulfonate), |
| | Compound of Formula I |
| XRPD | FIG. 1: Peaks at 6.3°, 17.8°, and 19.7° 2θ |
| DSC | FIG. 2: Endotherm extrapolated onset temperature: about |
| | 240.9° C. |
| DSC | FIG. 2: Peak temperature: about 243.8° C. |

Certain other XRPD peaks for (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I) are shown in Table 2 below.

TABLE 2
Selected X-Ray Powder Diffraction (XRPD) Peaks
for Compound of Formula 1 (Form 1)

| | | d- | Rel. | | | d- | Rel. |
|---|---|---|---|---|---|---|---|
| Pos. | Height | spacing | Int. | Pos. | Height | spacing | Int. |
| [°2θ] | [cts] | [Å] | [%] | [°2θ] | [cts] | [Å] | [%] |
| 5.3 | 6683 | 16.6 | 2.1 | 18.9 | 1727 | 4.7 | 0.5 |
| 5.6 | 1123 | 15.7 | 0.4 | 19.7 | 85818 | 4.5 | 26.4 |
| 6.3 | 325169 | 14.1 | 100.0 | 19.9 | 14907 | 4.5 | 4.6 |
| 7.7 | 1094 | 11.4 | 0.3 | 20.3 | 4687 | 4.4 | 1.4 |
| 8.5 | 1603 | 10.5 | 0.5 | 20.5 | 8017 | 4.3 | 2.5 |
| 9.7 | 3783 | 9.1 | 1.2 | 20.8 | 4692 | 4.3 | 1.4 |
| 10.7 | 5342 | 8.3 | 1.6 | 21.3 | 2197 | 4.2 | 0.7 |
| 11.3 | 2782 | 7.8 | 0.9 | 22.0 | 8093 | 4.0 | 2.5 |
| 11.4 | 5641 | 7.7 | 1.7 | 22.4 | 10630 | 4.0 | 3.3 |
| 12.1 | 2086 | 7.3 | 0.6 | 22.6 | 16934 | 3.9 | 5.2 |
| 12.5 | 5739 | 7.1 | 1.8 | 22.9 | 9728 | 3.9 | 3.0 |
| 12.8 | 5158 | 6.9 | 1.6 | 23.3 | 3477 | 3.8 | 1.1 |
| 12.9 | 1466 | 6.8 | 0.5 | 23.7 | 3607 | 3.7 | 1.1 |
| 13.8 | 5752 | 6.4 | 1.8 | 24.3 | 9676 | 3.7 | 3.0 |
| 13.9 | 2166 | 6.4 | 0.7 | 24.5 | 10358 | 3.6 | 3.2 |
| 15.0 | 1422 | 5.9 | 0.4 | 24.8 | 3818 | 3.6 | 1.2 |
| 15.5 | 12925 | 5.7 | 4.0 | 25.2 | 9020 | 3.5 | 2.8 |
| 16.0 | 5673 | 5.5 | 1.7 | 25.3 | 8090 | 3.5 | 2.5 |
| 16.5 | 19351 | 5.4 | 6.0 | 25.6 | 4722 | 3.5 | 1.5 |
| 16.8 | 6291 | 5.3 | 1.9 | 25.9 | 2572 | 3.4 | 0.8 |
| 17.0 | 7678 | 5.2 | 2.4 | 26.2 | 5545 | 3.4 | 1.7 |
| 17.8 | 12074 | 5.0 | 3.7 | 27.1 | 1248 | 3.3 | 0.4 |
| 18.1 | 6605 | 4.9 | 2.0 | 27.3 | 2056 | 3.3 | 0.6 |
| 18.3 | 14527 | 4.8 | 4.5 | 27.5 | 3976 | 3.2 | 1.2 |
| 18.4 | 9560 | 4.8 | 2.9 | 27.7 | 3474 | 3.2 | 1.1 |

One aspect of the present invention relates to a crystalline form of (2R,3R,11 bR)-3-isobutyl-9,10-dimethoxy-2,3,4,6,7,11 b-hexahydro-1H-pyrido[2,1-a]isoquinolin-2-yl (S)-2-amino-3-methylbutanoate di(4-methylbenzenesulfonate) (i.e., Compound of Formula I) that is substantially anhydrous. The anhydrous crystalline of (2R,3R,11 bR)-3-isobutyl-9,10-dimethoxy-2,3,4,6,7,11 b-hexahydro-1H-pyrido[2,1-a]isoquinolin-2-yl (S)-2-amino-3-methylbutanoate di(4-methylbenzenesulfonate) (i.e., Compound of Formula I) refers to a crystalline form that contains 2% or less of water. In some embodiments, the anhydrous crystalline form contains 100 or less water. In some embodiments, the water content is determined by Karl Fischer (KF) analysis.

One aspect of the present invention relates to a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I), wherein the crystalline form has an X-ray powder diffraction pattern comprising at least one peak, in terms of 2θ, selected from the group consisting of: 6.3°±0.2°, 15.5°±0.2°, 16.5°±0.2°, 17.8°±0.2°, 18.3°±0.2°, 19.7°±0.2°, 19.9°±0.2°, and 22.6°±0.2°; or selected from the group consisting of the peaks in Table 2. In some embodiments, the crystalline form of the compound 1 of Formula I has an X-ray powder diffraction pattern comprising at least two peaks, in terms of 2θ, selected from the group consisting of: 6.3°±0.2°, 15.5°±0.2°, 16.5°±0.2°, 17.8°±0.2°, 18.3°±0.2°, 19.7°±0.2°, 19.9°±0.2°, and 22.6°±0.2°; or selected from the group consisting of the peaks in Table 2. In some embodiments, the crystalline form of the compound 1 of Formula I has an X-ray powder diffraction pattern comprising at least three peaks, in terms of 2θ, selected from the group consisting of: 6.3°±0.2°, 15.5°±0.2°, 16.5°±0.2°, 17.8°±0.2°, 18.3°±0.2°, 19.7°±0.2°, 19.9°±0.2°, and 22.6°±0.2°; or selected from the group consisting of the peaks in Table 2.

In some embodiments, the crystalline form of the compound 1 of Formula I has an X-ray powder diffraction pattern comprising at least four peaks, in terms of 2θ, selected from the group consisting of: 6.3°±0.2°, 15.5°±0.2°, 16.5°±0.2°, 17.8°±0.2°, 18.3°±0.2°, 19.7°±0.2°, 19.9°±0.2°, and 22.6°±0.2°; or selected from the group consisting of the peaks in Table 2. In some embodiments, the crystalline form of the compound 1 of Formula I has an X-ray powder diffraction pattern comprising at least five peaks, in terms of 2θ, selected from the group consisting of: 6.3°±0.2°, 15.5°±0.2°, 16.5°±0.2°, 17.8°±0.2°, 18.3°±0.2°, 19.7°±0.2°, 19.9°±0.2°, and 22.6°±0.2°; or selected from the group consisting of the peaks in Table 2. In some embodiments, the crystalline form of the compound 1 of Formula I has an X-ray powder diffraction pattern comprising at least six peaks, in terms of 2θ, selected from the group consisting of: 6.3°±0.2°, 15.5°±0.2°, 16.5°±0.2°, 17.8°±0.2°, 18.3°±0.2°, 19.7°±0.2°, 19.9°±0.2°, and 22.6°±0.2°; or selected from the group consisting of the peaks in Table 2. In some embodiments, the crystalline form of the compound 1 of Formula I has an X-ray powder diffraction pattern comprising at least seven peaks, in terms of 2θ, selected from the group consisting of: 6.3°±0.2°, 15.5°±0.2°, 16.5°±0.2°, 17.8°±0.2°, 18.3°±0.2°, 19.7°±0.2°, 19.9°±0.2°, and 22.6°±0.2°; or selected from the group consisting of the peaks in Table 2.

One aspect of the present invention relates to a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., the compound of Formula I), wherein the crystalline form has an X-ray powder diffraction pattern comprising peaks, in terms of 2θ, at 6.3°±0.2°. In some embodiments, the crystalline form of the compound of Formula I has an X-ray powder diffraction pattern comprising peaks, in terms of 2θ, at 19.7°±0.2°. In some embodiments, the crystalline form of the compound of Formula I has an X-ray powder diffraction pattern comprising peaks, in terms of 2θ, at 17.8°±0.2°. In some embodiments, the crystalline form of the compound of Formula I has an X-ray powder diffraction pattern comprising peaks, in terms of 2θ, at 6.3°±0.2°, and 19.7°±0.2°. In some embodiments, the crystalline form of the compound of Formula I has an X-ray powder diffraction pattern comprising peaks, in terms of 2θ, at 6.3°±0.2°, and 17.8°±0.2°. In some embodiments, the crystalline form of the compound of Formula I has an X-ray powder diffraction pattern comprising peaks, in terms of 2θ, at 6.3°±0.2°, 17.8°±0.2°, and 19.7°±0.2°. In some embodiments, the crystalline form of the compound of Formula I has an X-ray powder diffraction pattern comprising peaks, in terms of 2θ, at 6.3°±0.2°, 16.5°±0.2°, 17.8°±0.2°, and 19.7°±0.2°. In some embodiments, the crystalline form of the compound of Formula I has an X-ray powder diffraction pattern comprising peaks, in terms of 2θ, at 6.3°±0.2°, 17.8°±0.2°, 19.7°±0.2°, and 22.6°±0.2°. In some embodiments, the crystalline form of the compound of Formula I has an X-ray powder diffraction pattern comprising peaks, in terms of 2θ, at 6.3°±0.2°, 17.8°±0.2°, 19.7°±0.2°, and 19.9°±0.2°. In some embodiments, the crystalline form of the compound of Formula I has an X-ray powder diffraction pattern comprising peaks, in terms of 2θ, at 6.3°±0.2°, 17.8°±0.2°, 18.3°±0.2°, and 19.7°±0.2°. In some embodiments, the crystalline form of the compound of Formula I has an X-ray powder diffraction pattern comprising peaks, in terms of 2θ, at 6.3°±0.2°, 15.5°±0.2°, 17.8°±0.2°, and 19.7°±0.2°. In some embodiments, the crystalline form of the compound of Formula I has an X-ray powder diffraction pattern comprising peaks, in terms of 2θ, at 6.3°±0.2°, 16.5°±0.2°, 17.8°±0.2°, 19.7°±0.2°, and 22.6°±0.2°. In some embodiments, the crystalline form of the compound of Formula I has an X-ray powder diffraction pattern comprising peaks, in terms of 2θ, at 6.3°±0.2°, 16.5°±0.2°, 17.8°±0.2°, 19.7°±0.2°, and 19.9°±0.2°. In some embodiments, the crystalline form of the compound of Formula I has an X-ray powder diffraction pattern comprising peaks, in terms of 20, at 6.3°±0.2°, 16.5°±0.2°, 17.8°±0.2°, 18.3°±0.2°, and 19.7°±0.2°. In some embodiments, the crystalline form of the compound of Formula I has an X-ray powder diffraction pattern comprising peaks, in terms of 2θ, at 6.3°±0.2°, 15.5°±0.2°, 16.5°±0.2°, 17.8°±0.2°, and 19.7°±0.2°. In some embodiments, the crystalline form of the compound of Formula I has an X-ray powder diffraction pattern comprising peaks, in terms of 2θ, at 6.3°±0.2°, 16.5°±0.2°, 17.8°±0.2°, 19.7°±0.2°, 19.9°±0.2°, and 22.6°±0.2°. In some embodiments, the crystalline form of the compound of Formula I has an X-ray powder diffraction pattern comprising peaks, in terms of 2θ, at 6.3°±0.2°, 16.5°±0.2°, 17.8°±0.2°, 18.3°±0.2°, 19.7°±0.2°, and 22.6°±0.2°. In some embodiments, the crystalline form of the compound of Formula I has an X-ray powder diffraction pattern comprising peaks, in terms of 2θ, at 6.3°±0.2°, 15.5°±0.2°, 16.5°±0.2°, 17.8°±0.2°, 19.7°±0.2°, and 22.6°±0.2°. In some embodiments, the crystalline form of the compound of Formula I has an X-ray powder diffraction pattern comprising peaks, in terms of 2θ, at 6.3°±0.2°, 16.5°±0.2°, 17.8°±0.2°, 18.3°±0.2°, 19.7°±0.2°, 19.9°±0.2°, and 22.6°±0.2°. In some embodiments, the crystalline form of the compound of Formula I has an X-ray powder diffraction pattern comprising peaks, in terms of 2θ, at 6.3°±0.2°, 15.5°±0.2°, 16.5°±0.2°, 17.8°±0.2°, 19.7°±0.2°, 19.9°±0.2°, and 22.6°±0.2°. In some embodiments, the crystalline form of the compound of Formula I has an X-ray powder diffraction pattern comprising peaks, in terms of 2θ, at 6.3°±0.2°, 15.5°±0.2°, 16.5°±0.2°, 17.8°±0.2°, 18.3°±0.2°, 19.7°±0.2°, 19.9°±0.2°, and 22.6°±0.2°. In some embodiments, the crystalline form of the compound of Formula I has an X-ray powder diffraction pattern substantially as shown in FIG. 1, wherein the word “substantially” is meant that the reported peaks can vary by about +0.2°20.

It is understood that peak intensities can vary from one diffractogram to another for the same crystalline form based on any number of factors that are known to those skilled in the art, such as, preferred orientation effects, preparation technique, the sample mounting procedure, the instrument employed, etc. In some instances, peak intensities can be rather dramatical. Accordingly, the diffraction peak intensities shown herein are illustrative and identical diffraction peak intensities are not necessarily required. Further, it is understood that those skilled in the art would readily be capable of comparing the diffractogram provided herein with a diffractogram generated for an unknown crystal form and confirm whether the diffractogram is characterizing the same crystal form as provided herein or a different form.

One aspect of the present invention relates to a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I), wherein the crystalline form has a differential scanning calorimetry (DSC) thermogram comprising an endotherm with an extrapolated onset temperature of about 237.9° C. to about 243.9° C. In some embodiments, the crystalline form of the compound of Formula I has a differential scanning calorimetry thermogram comprising an endotherm with an extrapolated onset temperature of about 238.4° C. to about 243.4° C. In some embodiments, the crystalline form of the compound of Formula I has a differential scanning calorimetry thermogram comprising an endotherm with an extrapolated onset temperature of about 238.9° C. to about 242.9° C. In some embodiments, the crystalline form of the compound of Formula I has a differential scanning calorimetry thermogram comprising an endotherm with an extrapolated onset temperature of about 239.4° C. to about 242.4° C. In some embodiments, the crystalline form of the compound of Formula I has a differential scanning calorimetry thermogram comprising an endotherm with an extrapolated onset temperature of about 239.9° C. to about 241.9° C. In some embodiments, the crystalline form of the compound of Formula I has a differential scanning calorimetry thermogram comprising an endotherm with an extrapolated onset temperature of about 240.4° C. to about 241.4° C.

One aspect of the present invention relates to a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I), wherein the crystalline form has a differential scanning calorimetry (DSC) thermogram comprising an endotherm with a peak temperature of about 240.8° C. to about 246.8° C. In some embodiments, the crystalline form of the compound of Formula I has a differential scanning calorimetry thermogram comprising an endotherm with a peak temperature of about 241.3° C. to about 246.3° C. In some embodiments, the crystalline form of the compound of Formula I has a differential scanning calorimetry thermogram comprising an endotherm with a peak temperature of about 241.8° C. to about 245.8° C. In some embodiments, the crystalline form of the compound of Formula I has a differential scanning calorimetry thermogram comprising an endotherm with a peak temperature of about 242.3° C. to about 245.3° C. In some embodiments, the crystalline form of the compound of Formula I has a differential scanning calorimetry thermogram comprising an endotherm with a peak temperature of about 242.8° C. to about 244.8° C. In some embodiments, the crystalline form of the compound of Formula I has a differential scanning calorimetry thermogram comprising an endotherm with a peak temperature of about 243.3° C. to about 244.3° C.

One aspect of the present invention relates to a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I), wherein the crystalline form has a differential scanning calorimetry (DSC) thermogram comprising an endotherm with an extrapolated onset temperature of about 237.9° C. to about 243.9° C. and a peak temperature of about 240.8° C. to about 246.8° C. In some embodiments, the crystalline form of the compound of Formula I has a differential scanning calorimetry thermogram comprising an endotherm with an extrapolated onset temperature of about 238.4° C. to about 243.4° C. and a peak temperature of about 241.3° C. to about 246.3° C. In some embodiments, the crystalline form of the compound of Formula I has a differential scanning calorimetry thermogram comprising an endotherm with an extrapolated onset temperature of about 238.9° C. to about 242.9° C. and a peak temperature of about 241.8° C. to about 245.8° C. In some embodiments, the crystalline form of the compound of Formula I has a differential scanning calorimetry thermogram comprising an endotherm with an extrapolated onset temperature of about 239.4° C. to about 242.4° C. and a peak temperature of about 242.3° C. to about 245.3° C. In some embodiments, the crystalline form of the compound of Formula I has a differential scanning calorimetry thermogram comprising an endotherm with an extrapolated onset temperature of about 239.9° C. to about 241.9° C. and a peak temperature of about 242.8° C. to about 244.8° C. In some embodiments, the crystalline form of the compound of Formula I has a differential scanning calorimetry thermogram comprising an endotherm with an extrapolated onset temperature of about 240.4° C. to about 241.4° C. and a peak temperature of about 243.3° C. to about 244.3° C. In some embodiments, the crystalline form of the compound of Formula I has a differential scanning calorimetry thermogram substantially as shown in FIG. 2, wherein the word “substantially” is meant that the reported DSC features can vary by about ±5° C.

One aspect of the present invention relates to a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I), wherein the crystalline form has:

an X-ray powder diffraction pattern comprising at least one peak, in terms of 2θ, selected from the group consisting of: 6.3°±0.2°, 15.5°±0.2°, 16.5°±0.2°, 17.8°±0.2°, 18.3°±0.2°, 19.7°±0.2°, 19.90 0.20, and 22.60 0.20;

a differential scanning calorimetry (DSC) thermogram comprising an endotherm with an extrapolated onset temperature of about 237.9° C. to about 243.9° C.; and/or

a differential scanning calorimetry (DSC) thermogram comprising an endotherm with a peak temperature of about 240.8° C. to about 246.8° C.

One aspect of the present invention relates to a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I), wherein the crystalline form has: an X-ray powder diffraction pattern comprising at least two peaks, in terms of 2θ, selected from the group consisting of: 6.3°±0.2°, 15.5°±0.2°, 16.5°±0.2°, 17.8°±0.2°, 18.3°±0.2°, 19.7°±0.2°, 19.90 0.20, and 22.60 0.20;

a differential scanning calorimetry thermogram comprising an endotherm with an extrapolated onset temperature of about 238.4° C. to about 243.4° C.; and/or

a differential scanning calorimetry thermogram comprising an endotherm with a peak temperature of about 241.3° C. to about 246.3° C.

One aspect of the present invention relates to a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I), wherein the crystalline form has:

an X-ray powder diffraction pattern comprising at least three peaks, in terms of 2θ, selected from the group consisting of: 6.3°±0.2°, 15.5°±0.2°, 16.5°±0.2°, 17.8°±0.2°, 18.3°±0.2°, 19.7°±0.2°, 19.90 0.20, and 22.60 0.20;

a differential scanning calorimetry thermogram comprising an endotherm with an extrapolated onset temperature of about 238.9° C. to about 242.9° C.; and/or

a differential scanning calorimetry thermogram comprising an endotherm with a peak temperature of about 241.8° C. to about 245.8° C.

One aspect of the present invention relates to a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I), wherein the crystalline form has:

an X-ray powder diffraction pattern comprising peaks, in terms of 2θ, at 6.3°±0.2°;

a differential scanning calorimetry (DSC) thermogram comprising an endotherm with an extrapolated onset temperature of about 237.9° C. to about 243.9° C.; and/or

a differential scanning calorimetry (DSC) thermogram comprising an endotherm with a peak temperature of about 240.8° C. to about 246.8° C.

One aspect of the present invention relates to a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I), wherein the crystalline form has:

an X-ray powder diffraction pattern comprising peaks, in terms of 2θ, at 6.3°±0.2°, and 19.7°±0.20;

a differential scanning calorimetry thermogram comprising an endotherm with an extrapolated onset temperature of about 238.4° C. to about 243.4° C.; and/or

a differential scanning calorimetry thermogram comprising an endotherm with a peak temperature of about 241.3° C. to about 246.3° C.

One aspect of the present invention relates to a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I), wherein the crystalline form has:

an X-ray powder diffraction pattern comprising peaks, in terms of 2θ, at 6.3°±0.2°, 17.8°±0.2°, and 19.7°±0.2°;

a differential scanning calorimetry thermogram comprising an endotherm with an extrapolated onset temperature of about 238.9° C. to about 242.9° C.; and/or

a differential scanning calorimetry thermogram comprising an endotherm with a peak temperature of about 241.8° C. to about 245.8° C.

One aspect of the present invention relates to a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I), wherein the crystalline form has:

an X-ray powder diffraction pattern comprising peaks, in terms of 2θ, at 6.3°±0.2°, 16.5°±0.2°, 17.80 0.20, 19.70 0.20, and 22.60 0.20;

a differential scanning calorimetry thermogram comprising an endotherm with an extrapolated onset temperature of about 239.4° C. to about 242.4° C.; and/or

a differential scanning calorimetry thermogram comprising an endotherm with a peak temperature of about 242.3° C. to about 245.3° C.

One aspect of the present invention relates to a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I), wherein the crystalline form has:

an X-ray powder diffraction pattern comprising peaks, in terms of 2θ, at 6.3°±0.2°, 16.5°±0.2°, 17.80 0.20, 19.70 0.20, 19.90 0.20, and 22.60°±0.20;

a differential scanning calorimetry thermogram comprising an endotherm with an extrapolated onset temperature of about 239.9° C. to about 241.9° C.; and/or

a differential scanning calorimetry thermogram comprising an endotherm with a peak temperature of about 242.8° C. to about 244.8° C.

One aspect of the present invention relates to a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I), wherein the crystalline form has:

an X-ray powder diffraction pattern comprising peaks, in terms of 2θ, at 6.3°±0.2°, 16.5°±0.2°, 17.80 0.20, 18.30 0.20, 19.70 0.20, 19.90 0.20, and 22.60°±0.20;

a differential scanning calorimetry thermogram comprising an endotherm with an extrapolated onset temperature of about 240.4° C. to about 241.4° C.; and/or

a differential scanning calorimetry thermogram comprising an endotherm with a peak temperature of about 243.3° C. to about 244.3° C.

One aspect of the present invention relates to a crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., the compound of Formula I), wherein the crystalline form has:

an X-ray powder diffraction pattern substantially as shown in FIG. 1; and/or

a differential scanning calorimetry thermogram substantially as shown in FIG. 2.

In some embodiments, the crystalline form of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., the compound of Formula I) can be isolated as the crystalline form described herein, with a crystalline purity of at least about 75% by weight. In some embodiments, about 80% by weight. In some embodiments, about 85% by weight. In some embodiments, about 90% by weight. In some embodiments, about 95% by weight. In some embodiments, about 96% by weight. In some embodiments, about 97% by weight. In some embodiments, about 98% by weight. In some embodiments, about 99% by weight.

Manufacturing batches of(2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., the compound of Formula I) have been prepared in a similar manner as described herein and have the following particle size distribution characterizations, as shown in Table 3.

TABLE 3

| Particle size | Batch | Batch | Batch | Batch | Batch |
|---|---|---|---|---|---|
| distribution | 073 ¹ | 084 ² | 085 ² | 086 ² | 087 ² |
| D10 | 3 μM | 3 μM | 3 μM | 3 μM | 3 μM |
| D50 | 14 μM | 13 μM | 13 μM | 14 μM | 13 μM |
| D90 | 45 μM | 43 μM | 44 μM | 48 μM | 45 μM |

¹ Batch 073 was prepared as described in Example 1, Step E.
² Batch prepared in a similar a manner as described in Example 1, Step E.

In some embodiments, the crystalline form of 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine (Compound 1, free base) has a particle size D10 of about 1 μM to about 8 μM. In some embodiments, the crystalline form of the compound of Formula I has a particle size D10 of about 1 μM to about 7 μM. In some embodiments, the crystalline form of the compound of Formula I has a particle size D10 of about 2 μM to about 6 μM. In some embodiments, the crystalline form of the compound of Formula I has a particle size D10 of about 2 μM to about 5 μM. In some embodiments, the crystalline form of the compound of Formula I has a particle size D10 of about 2 μM to about 4 μM.

In some embodiments, the crystalline form of 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine (Compound 1, free base) has a particle size D50 of about 4 μM to about 27 μM. In some embodiments, the crystalline form of the compound of Formula I has a particle size D50 of about 6 μM to about 20 μM. In some embodiments, the crystalline form of the compound of Formula I has a particle size D50 of about 8 μM to about 18 μM. In some embodiments, the crystalline form of the compound of Formula I has a particle size D50 of about 10 μM to about 16 μM. In some embodiments, the crystalline form of the compound of Formula I has a particle size D50 of about 12 μM to about 15 μM.

In some embodiments, the crystalline form of 4-(2-chloro-4-methoxy-5-methylphenyl)-N-[(1S)-2-cyclopropyl-1-(3-fluoro-4-methylphenyl)ethyl]-5-methyl-N-prop-2-ynyl-1,3-thiazol-2-amine (Compound 1, free base) has a particle size D90 of about 19 μM to about 62 μM. In some embodiments, the crystalline form of the compound of Formula I has a particle size D90 of about 28 μM to about 58 μM. In some embodiments, the crystalline form of the compound of Formula I has a particle size D90 of about 35 μM to about 55 μM. In some embodiments, the crystalline form of the compound of Formula I has a particle size D90 of about 40 μM to about 51 μM. In some embodiments, the crystalline form of the compound of Formula I has a particle size D90 of about 41 μM to about 50 μM.

Pharmaceutical Compositions and Pharmaceutical Products comprising the Compound of Formula I.

One aspect of the present invention relates to pharmaceutical compositions comprising a (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (i.e., Compound of Formula I) and a pharmaceutically acceptable carrier. In some embodiments, the pharmaceutical composition is adapted for oral administration. In some embodiments, the pharmaceutical composition is in the form of a tablet or capsule. In some embodiments, the pharmaceutical composition is in the form of a tablet. In some embodiments, the pharmaceutical composition is in the form of a capsule.

One aspect of the present invention relates to pharmaceutical products selected from: a pharmaceutical composition, a formulation, a unit dosage form, and a kit; each comprising a (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (Compound of Formula I) composition as described herein.

One aspect of the present invention relates to processes for preparing a pharmaceutical compositions comprising admixing a (2R,3R,11 bR)-3-isobutyl-9,10-dimethoxy-2,3,4,6,7,11b-hexahydro-1H-pyrido[2,1-a]isoquinolin-2-yl (5)-2-amino-3-methylbutanoate di(4-methylbenzenesulfonate) (Compound of Formula I) composition as described herein, and a pharmaceutically acceptable carrier.

One aspect of the present invention relates to processes for preparing pharmaceutical compositions comprising admixing the crystal form of (2R,3R,11 bR)-3-isobutyl-9,10-dimethoxy-2,3,4,6,7,11b-hexahydro-1H-pyrido[2,1-a]isoquinolin-2-yl (5)-2-amino-3-methylbutanoate di(4-methylbenzenesulfonate) (Compound of Formula I) with a pharmaceutically acceptable carrier, wherein the anhydrous crystalline form is prepared by any of the processes described herein.

Compositions Comprising the Compound of Formula I.

One aspect of the present invention relates to compositions comprising:

a. (2R,3R,11bR)-3-isobutyl-9,10-dimethoxy-2,3,4,6,7,11b-hexahydro-1H-pyrido[2,1-a]isoquinolin-2-yl (5)-2-amino-3-methylbutanoate di(4-methylbenzenesulfonate) (Compound of Formula I); and

b. at least one compound selected from:

### (2R,3R,11bR)-9,10-dimethoxy-3-(2-methylpropyl)-1H,2H,3H,4H,6H,7H,11bH-pyrido[2,1-a]isoquinolin-2-yl (2S)-2-aminopropanoate (Compound 2A)

(2R,3R,11bR)-9,10-dimethoxy-3-(2-methylpropyl)-1H,2H,3H,4H,6H,7H,11bH-pyrido[2,1-a]isoquinolin-2-ol (Compound 2B);

### 3-isobutyl-9,10-dimethoxy-6,7-dihydropyrido[2,1-a]isoquinolin-5-ium salt (Compound 2C)

(2R,3R,11bR)-9,10-dimethoxy-3-(2-methylpropyl)-7-oxo-1H,2H,3H,4H,6H,7H,11bH-pyrido[2,1-a]isoquinolin-2-yl (2S)-2-amino-3-methylbutanoate (Compound 2D);

### (2R,3R,11bR)-9,10-dimethoxy-3-(2-methylpropyl)-1H,2H,3H,4H,6H,7H,11bH-pyrido[2,1-a]isoquinolin-2-yl (2R)-2-amino-3-methylbutanoate (Compound 2E)

acetonitrile;

ethanol;

dichloromethane; and

methanol.

In some embodiments, the composition comprising the compound of Formula I has at least two compounds selected from: (2R,3R,11 bR)-9,10-dimethoxy-3-(2-methylpropyl)-1H,2H,3H,4H,6H,7H,11 bH-pyrido[2,1-a]isoquinolin-2-yl (2S)-2-aminopropanoate (Compound 2A); (2R,3R,11bR)-9,10-dimethoxy-3-(2-methylpropyl)-1H,2H,3H,4H,6H,7H,11bH-pyrido[2,1-a]isoquinolin-2-ol (Compound 2B); 3-isobutyl-9,10-dimethoxy-6,7-dihydropyrido[2,1-a]isoquinolin-5-ium salt (Compound 2C); (2R,3R,11bR)-9,10-dimethoxy-3-(2-methylpropyl)-7-oxo-1H,2H,3H,4H,6H,7H,11bH-pyrido[2,1-a]isoquinolin-2-yl (2S)-2-amino-3-methylbutanoate (Compound 2D); (2R,3R,11bR)-9,10-dimethoxy-3-(2-methylpropyl)-1H,2H,3H,4H,6H,7H,11bH-pyrido[2,1-a]isoquinolin-2-yl (2R)-2-amino-3-methylbutanoate (Compound 2E); acetonitrile; ethanol; dichloromethane; and methanol (for brevity, the above group of compounds is referred to as the “list” in this paragraph). In some embodiments, the composition comprises the compound of Formula I and at least three compounds selected from the “list”.

In some embodiments, the composition comprises the compound of Formula I and has at least four compounds selected from the “list”. In some embodiments, the composition comprises the compound of Formula I and has at least five compounds selected from the “list”. In some embodiments, the composition comprises the compound of Formula I and has at least six compounds selected from the “list”. In some embodiments, the composition comprises the compound of Formula I and has at least seven compounds selected from the “list”. In some embodiments, the composition comprises the compound of Formula I and has at least eight compounds selected from the “list”.

In some embodiments, the composition contains at least 97% of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (Compound of Formula I) as determined by HPLC. In some embodiments, the composition contains at least 98% of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (Compound of Formula I) as determined by HPLC. In some embodiments, the composition contains at least 99% of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (Compound of Formula I) as determined by HPLC.

In some embodiments, the composition contains no more than 0.3% of (2R,3R,11bR)-9,10-dimethoxy-3-(2-methylpropyl)-1H,2H,3H,4H,6H,7H,11bH-pyrido[2,1-a]isoquinolin-2-yl (2S)-2-aminopropanoate (Compound 2A) as determined by HPLC. In some embodiments, the composition contains no more than 0.2% of (2R,3R,11bR)-9,10-dimethoxy-3-(2-methylpropyl)-1H,2H,3H,4H,6H,7H,11bH-pyrido[2,1-a]isoquinolin-2-yl (2S)-2-aminopropanoate (Compound 2A) as determined by HPLC. In some embodiments, the composition contains no more than 0.1% of (2R,3R,11bR)-9,10-dimethoxy-3-(2-methylpropyl)-1H,2H,3H,4H,6H,7H,11bH-pyrido[2,1-a]isoquinolin-2-yl (2S)-2-aminopropanoate (Compound 2A) as determined by HPLC.

In some embodiments, the composition contains no more than 0.3% of (2R,3R,11bR)-9,10-dimethoxy-3-(2-methylpropyl)-1H,2H,3H,4H,6H,7H,11bH-pyrido[2,1-a]isoquinolin-2-ol (Compound 2B) as determined by HPLC. In some embodiments, the composition contains no more than 0.2% of (2R,3R,11bR)-9,10-dimethoxy-3-(2-methylpropyl)-1H,2H,3H,4H,6H,7H,11bH-pyrido[2,1-a]isoquinolin-2-ol (Compound 2B) as determined by HPLC. In some embodiments, the composition contains no more than 0.1% of(2R,3R,11bR)-9,10-dimethoxy-3-(2-methylpropyl)-1H,2H,3H,4H,6H,7H,11 bH-pyrido[2,1-a]isoquinolin-2-ol (Compound 2B) as determined by HPLC.

In some embodiments, the composition contains no more than 0.2% of 3-isobutyl-9,10-dimethoxy-6,7-dihydropyrido[2,1-a]isoquinolin-5-ium salt (Compound 2C) as determined by HPLC. In some embodiments, the composition contains no more than 0.1% of 3-isobutyl-9,10-dimethoxy-6,7-dihydropyrido[2,1-a]isoquinolin-5-ium salt (Compound 2C) as determined by HPLC. In some embodiments, the composition contains no more than 0.05% of 3-isobutyl-9,10-dimethoxy-6,7-dihydropyrido[2,1-a]isoquinolin-5-ium salt (Compound 2C) as determined by HPLC.

In some embodiments, the composition contains no more than 0.3% of (2R,3R,11 bR)-9,10-dimethoxy-3-(2-methylpropyl)-7-oxo-1H,2H,3H,4H,6H,7H,11bH-pyrido[2,1-a]isoquinolin-2-yl (2S)-2-amino-3-methylbutanoate (Compound 2D) as determined by HPLC. In some embodiments, the composition contains no more than 0.2% of (2R,3R,11bR)-9,10-dimethoxy-3-(2-methylpropyl)-7-oxo-1H,2H,3H,4H,6H,7H,11bH-pyrido[2,1-a]isoquinolin-2-yl (2S)-2-amino-3-methylbutanoate (Compound 2D) as determined by HPLC. In some embodiments, the composition contains no more than 0.1% of (2R,3R,11bR)-9,10-dimethoxy-3-(2-methylpropyl)-7-oxo-1H,2H,3H,4H,6H,7H,11bH-pyrido[2,1-a]isoquinolin-2-yl (2S)-2-amino-3-methylbutanoate (Compound 2D) as determined by HPLC.

In some embodiments, the composition contains no more than 0.5% of (2R,3R,11bR)-9,10-dimethoxy-3-(2-methylpropyl)-1H,2H,3H,4H,6H,7H,11bH-pyrido[2,1-a]isoquinolin-2-yl (2R)-2-amino-3-methylbutanoate (Compound 2E) as determined by HPLC. In some embodiments, the composition contains no more than 0.4% of (2R,3R,11bR)-9,10-dimethoxy-3-(2-methylpropyl)-1H,2H,3H,4H,6H,7H,11bH-pyrido[2,1-a]isoquinolin-2-yl (2R)-2-amino-3-methylbutanoate (Compound 2E) as determined by HPLC. In some embodiments, the composition contains no more than 0.3% of (2R,3R,11bR)-9,10-dimethoxy-3-(2-methylpropyl)-1H,2H,3H,4H,6H,7H,11bH-pyrido[2,1-a]isoquinolin-2-yl (2R)-2-amino-3-methylbutanoate (Compound 2E) as determined by HPLC. In some embodiments, the composition contains no more than 0.2% of (2R,3R,11bR)-9,10-dimethoxy-3-(2-methylpropyl)-1H,2H,3H,4H,6H,7H,11bH-pyrido[2,1-a]isoquinolin-2-yl (2R)-2-amino-3-methylbutanoate (Compound 2E) as determined by HPLC. In some embodiments, the composition contains no more than 0.1% of (2R,3R,11bR)-9,10-dimethoxy-3-(2-methylpropyl)-1H,2H,3H,4H,6H,7H,11bH-pyrido[2,1-a]isoquinolin-2-yl (2R)-2-amino-3-methylbutanoate (Compound 2E) as determined by HPLC.

In some embodiments, the composition contains no more than 410 ppm of acetonitrile as determined by gas chromatography. In some embodiments, the composition contains no more than 300 ppm of acetonitrile as determined by gas chromatography. In some embodiments, the composition contains no more than 100 ppm of acetonitrile as determined by gas chromatography. In some embodiments, the composition contains no more than 50 ppm of acetonitrile as determined by gas chromatography.

In some embodiments, the composition contains no more than 5000 ppm of ethanol as determined by gas chromatography. In some embodiments, the composition contains no more than 3000 ppm of ethanol as determined by gas chromatography. In some embodiments, the composition contains no more than 1000 ppm of ethanol as determined by gas chromatography. In some embodiments, the composition contains no more than 100 ppm of ethanol as determined by gas chromatography.

In some embodiments, the composition contains no more than 600 ppm of dichloromethane as determined by gas chromatography. In some embodiments, the composition contains no more than 400 ppm of dichloromethane as determined by gas chromatography. In some embodiments, the composition contains no more than 100 ppm of dichloromethane as determined by gas chromatography. In some embodiments, the composition contains no more than 30 ppm of dichloromethane as determined by gas chromatography.

In some embodiments, the composition contains no more than 3000 ppm of methanol as determined by gas chromatography. In some embodiments, the composition contains no more than 1000 ppm of methanol as determined by gas chromatography. In some embodiments, the composition contains no more than 500 ppm of methanol as determined by gas chromatography. In some embodiments, the composition contains no more than 60 ppm of methanol as determined by gas chromatography.

In some embodiments, (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (Compound of Formula I) is crystalline. In some embodiments, (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (Compound of Formula I) is Form I as described herein.

Abbreviations/Definitions

As used herein “stable” refers to a compound that is sufficiently robust to survive isolation to a useful degree of purity from a reaction mixture, and preferably capable of formulation into an efficacious therapeutic agent.

As used in the specification and the accompanying claims, the indefinite articles “a” and “an” and the definite article “the” include plural as well as singular referents, unless the context clearly dictates otherwise.

The term “about” or “approximately” means an acceptable error for a particular value as determined by one of ordinary skill in the art, which depends in part on how the value is measured or determined. In some embodiments, the term “about” or “approximately” means within 1, 2, 3, or 4 standard deviations. In some embodiments, the term “about” or “approximately” means within 30%, 25%, 20%, 15%, 10%, 9%, 8%, 7%, 6%, 5%, 4%, 3%, 2%, 1%, 0.5% or 0.05% of a given value or range.

The term “crystalline form” of a compound can refer to any crystalline form of the compound as a free acid, the compound as a free base, as an acid addition salt of the compound, an base addition salt of the compound, a complex of the compound, a solvate (including hydrate) of the compound, or a co-crystal of the compound. The term “solid form” of a compound can refer to any crystalline form of the compound or any amorphous form of the compound as a free acid, the compound as a free base, as an acid addition salt of the compound, an base addition salt of the compound, a complex of the compound, or a solvate (including hydrate) of the compound, or a co-precipitation of the compound. In many instances, the terms “crystalline form” and “solid form” can refer to those that are pharmaceutically acceptable, including, for example, those of pharmaceutically acceptable addition salts, pharmaceutically acceptable complexes, pharmaceutically acceptable solvates, pharmaceutically acceptable co-crystals, and pharmaceutically acceptable co-precipitations.

The terms “process” and “method” are used interchangeably to refer to a method disclosed herein for a compound preparation. Modifications to the processes and methods disclosed herein (e.g., starting materials, reagents, protecting groups, solvents, temperatures, reaction times, and/or purification) that are well known to those of ordinary skill in the art are also encompassed by the disclosure.

The terms “adding”, “reacting” and “mixing” are used interchangeably to refer to contacting one reactant, reagent, solvent, catalyst, or a reactive group with another reactant, reagent, solvent, catalyst, or reactive group. Unless otherwise specified, reactants, reagents, solvents, catalysts, and reactive groups can be added individually, simultaneously, or separately, and/or can be added in any order They can be added in the presence or absence of heat, and can optionally be added under an inert atmosphere (e.g., N₂ or Ar).

In some embodiments, the term “reacting” can also refer to in situ formation or intra-molecular reaction where the reactive groups are in the same molecule.

It is further appreciated that certain features, which are, for clarity, described in the context of separate embodiments, can also be provided in combination in a single embodiment. Conversely, various features which are, for brevity, described in the context of a single embodiment, can also be provided separately or in any suitable subcombination.

Compounds disclosed herein can also include all isotopes of atoms occurring in the intermediates or final compounds. Isotopes include those atoms having the same atomic number but different mass numbers. For example, isotopes of hydrogen include tritium and deuterium.

In some embodiments, the compounds disclosed herein, and salts thereof, are substantially isolated. By “substantially isolated” is meant that the compound is at least partially or substantially separated from the environment in which it was formed or detected. Partial separation can include, for example, a composition enriched in a compound disclosed herein. Substantial separation can include compositions containing at least about 50%, at least about 60%, at least about 70%, at least about 80%, at least about 90%, at least about 95%, at least about 97%, or at least about 99% by weight of a compound disclosed herein, or salt thereof. Methods for isolating compounds and their salts are routine in the art.

The present application also includes salts of the compounds described herein. As used herein, “salts” refers to derivatives of the disclosed compounds wherein the parent compound is modified by converting an existing acid or base moiety to its salt form. Examples of salts include, but are not limited to, mineral acid (such as HCl, HBr, H₂SO₄) or organic acid (such as acetic acid, benzoic acid, trifluoroacetic acid) salts of basic residues such as amines; alkali (such as Li, Na, K, Mg, Ca) or organic (such as trialkylammonium) salts of acidic residues such as carboxylic acids; and the like. The salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile (ACN) are preferred.

The present application also includes pharmaceutically acceptable salts of the compounds described herein. The “pharmaceutically acceptable salts” include a subset of the “salts” described above which are, conventional non-toxic salts of the parent compound formed, for example, from non-toxic inorganic or organic acids. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The processes described herein can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry; or by chromatography such as high performance liquid chromatography (HPLC) or thin layer chromatography. The compounds obtained by the reactions can be purified by any suitable method known in the art. For example, chromatography (medium pressure) on a suitable adsorbent (e.g., silica gel, alumina and the like), HPLC, or preparative thin layer chromatography; distillation; sublimation, trituration, or recrystallization. The purity of the compounds, in general, are determined by physical methods such as measuring the melting point (in case of a solid), obtaining an NMR spectrum, or performing a HPLC separation. If the melting point decreases, if unwanted signals in the NMR spectrum are decreased, or if extraneous peaks in an HPLC trace are removed, the compound can be said to have been purified. In some embodiments, the compounds are substantially purified.

Preparation of compounds can involve the protection and deprotection of various chemical groups. The need for protection and deprotection, and the selection of appropriate protecting groups can be readily determined by one skilled in the art. The chemistry of protecting groups can be found, for example, in Wuts and Greene, Greene's Protective Groups in Organic Synthesis, 4^{th} Ed., John Wiley & Sons: New York, 2006, which is incorporated herein by reference in its entirety.

The reactions of the processes described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the reaction step, suitable solvent(s) for that particular reaction step can be selected. Appropriate solvents include water, alkanes (such as pentanes, hexanes, heptanes, cyclohexane, etc., or a mixture thereof), aromatic solvents (such as benzene, toluene, xylene, etc.), alcohols (such as methanol, ethanol, isopropanol, etc.), ethers (such as dialkylethers, methyl tert-butyl ether (MTBE), tetrahydrofuran (THF), dioxane, etc.), esters (such as ethyl acetate, butyl acetate, etc.), halogenated hydrocarbon solvents (such as dichloromethane (DCM), chloroform, dichloroethane, tetrachloroethane), dimethylformamide (DMF), dimethylsulfoxide (DMSO), acetone, acetonitrile (ACN), hexamethylphosphoramide (HMPA) and N-methyl pyrrolidone (NMP). Such solvents can be used in either their wet or anhydrous forms.

Resolution of racemic mixtures of compounds can be carried out by any of numerous methods known in the art. An example method includes fractional recrystallization using a “chiral resolving acid” which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids. Resolution of racemic mixtures can also be carried out by elution on a column packed with an optically active resolving agent (e.g., dinitrobenzoylphenylglycine). Suitable elution solvent composition can be determined by one skilled in the art.

Crystals used for seeding can be obtained, e.g., from the previous synthesis as described in U.S. Pat. No. 10,160,757 B2.

### EXAMPLES

The disclosure will be described in greater detail by way of specific examples. The following examples are offered for illustrative purposes and are not intended to limit the disclosure in any manner. Those of skill in the art will readily recognize a variety of non-critical parameters which can be changed or modified to yield essentially the same results.

In the examples below, unless otherwise indicated, all temperatures are set forth in degrees Celsius and all parts and percentages are by weight. Reagents may be purchased from commercial suppliers, such as, e.g., Sigma-Aldrich® Chemical Co., and may be used without further purification unless otherwise indicated. Reagents may also be prepared following standard literature procedures known to those skilled in the art. Solvents may be purchased, for example, from Sigma-Aldrich®, and may be used as received or may be purified using standard methods known to those skilled in the art, unless otherwise indicated.

Unless otherwise specified, the reactions set forth below were done generally at ambient temperature or room temperature. Reactions were assayed by HPLC and terminated as judged by the consumption of starting material.

The compound structures and purities in the examples below were confirmed by one or more of the following methods: proton nuclear magnetic resonance (¹H NMR) spectroscopy, ¹³C NMR spectroscopy, mass spectroscopy, infrared spectroscopy, melting point, X-ray crystallography, and/or HPLC. ¹H NMR spectra were determined using an NMR spectrometer operating at a certain field strength. Chemical shifts are reported in parts per million (ppm, 8) downfield from a standard, e.g., an internal standard, such as TMS. Alternatively, ¹H NMR spectra were referenced to signals from residual protons in deuterated solvents as follows: CDCl₃=7.26 ppm; DMSOd₆=2.50 ppm; C₆D₆=7 0.16 ppm; CD₃OD=3 0.31 ppm (J Org. Chem. 1997, 62, 7513). Peak multiplicities are designated as follows: s, singlet; d, doublet; dd, doublet of doublets; t, triplet; dt, doublet of triplets; q, quartet; br, broadened; and m, multiplet Coupling constants are given in Hertz (Hz). Mass spectra (MS) data were obtained using a mass spectrometer with APCI or ESI ionization.

### Example 1. Synthesis of (2R,3R,11bR)-3-Isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (Formula I)

Step A: Synthesis of 3-Isobutyl-9,10-dimethoxy-3,4,6,7-tetrahydro-1H-pyrido[2,1-a]isoquinolin-2(11bH)-one (Formula F4, see FIG. 3 and FIG. 7).

Charge demineralized water (231 L, 6.30 V), 3-((dimethylamino)methyl)-5-methylhexan-2-one oxalate (Formula F1,52.6 kg, 202 mol; 1.25 equiv) and tert-butyl methylether (95 L, 2.60 V) into reactor A. Heat to about 22° C. and adjust pH to 11 with a 10 wt % potassium hydroxide solution (210.8 kg, 376 mol, 2.33 equiv). Stir for no less than (“NLT”) 15 min and split the layers. Wash the organic layer with demineralized water (39 L, 1.05 V). Swap the solvent to isopropanol by put and take distillation at 1.50 V with isopropanol (129 L, 3.50 V). Cool to about 22° C. (19 to 25° C.) and charge demineralized water (55 L, 1.50 V), sodium iodide (9.7 kg, 65 mol, 0.40 equiv.), 6,7-dimethoxy-3,4-dihydroisoquinoline hydrochloride (Formula F3,36.7 kg, 161 mmol, 1.00 equiv). Heat to about 42° C. and stir for NLT 24h. Cool to about 22° C. and stir for NLT 1 h. Isolate the solid by filtration. Wash the cake with isopropanol (91.8 L, 2.50 V). Dry the wet 3-isobutyl-9,10-dimethoxy-3,4,6,7-tetrahydro-1H-pyrido[2,1-a]isoquinolin-2(11bH)-one (Formula F4) product at about 40° C. under vacuum for NLT 12 h. Yield: 45.3 kg, 143 mol, 88.5%, with 99.2% purity.

Step B: Synthesis of 3-Isobutyl-9,10-dimethoxy-2,3,4,6,7,11b-hexahydro-1H-pyrido[2,1-a]isoquinolin-2-ol (Formula F5, see FIG. 4 and FIG. 8).

Charge 3-isobutyl-9,10-dimethoxy-3,4,6,7-tetrahydro-1H-pyrido[2,1-a]isoquinolin-2(11bH)-one (Formula F4,44.3 kg, 139 mol, 1.00 equiv), tert-butyl methylether (195 L, 4.40 V), acetic acid (9.3 kg, 155 mol, 1.11 equiv) and methanol (44 L, 1.00 V) into reactor A. Charge a suspension of sodium borohydride (10.5 kg, 279 mol, 2.00 equiv) in tert-butyl methylether (44 L, 1.00 V) keeping the temperature at about 22° C. Rinse the preparation vessel and the transfer line with tert-butyl methylether (2×13 L, 2×0.30 V). Stir at about 25° C. for 2 h. Add a 1 N sodium hydroxide solution (230 kg, 222 mol, 1.59 equiv) at about 25° C. Heat to about 47° C. and stir for about 3 h. Cool to about 15° C. and stir for about 30 min. Isolate the solid by filtration. Wash the cake with water (4×44 L, 4×1.00 V) and tert-butyl methylether (44 L, 1.00 V). Dry the wet 3-isobutyl-9,10-dimethoxy-2,3,4,6,7,11b-hexahydro-1H-pyrido[2,1-a]isoquinolin-2-ol (Formula F5) product at about 40° C. under vacuum for NLT 12 h. Yield: 35.6 kg, 111 mol, 80.1% with 99.0% purity.

Step C: Synthesis of (2R,3R,11bR)-3-Isobutyl-9,10-dimethoxy-2,3,4,6,7,11b-hexahydro-1H-pyrido[2,1-a]isoquinolin-2-ol (S)-(+)-camphorsulfonate (Formula F6-CSA, see FIG. 4 and FIG. 8).

Charge absolute ethanol (428 L, 12.00 V), camphor D-(+)-sulfonic acid (21.4 kg, 92 mol, 0.825 equiv), 3-isobutyl-9,10-dimethoxy-2,3,4,6,7,11b-hexahydro-1H-pyrido[2,1-a]isoquinolin-2-ol (Formula F5,35.7 kg, 112 mol, 1.00 equiv), demineralized water (0.75 V) into reactor A. Heat to about 70° C. and stir for about 30 min. Cool to about 22° C. at about 3° C./h. Ensure that the product has crystallized. If not, seed with F6 CSA (0.2 kg, 0.5 wt %). Cool to about 22° C. at about 3° C./h and stir for about 2 h. Isolate the solid by filtration. Wash the cake with absolute ethanol (36 L, 1.00 V). Dry the wet (2R,3R,11bR)-3-isobutyl-9,10-dimethoxy-2,3,4,6,7,11b-hexahydro-1H-pyrido[2,1-a]isoquinolin-2-ol (S)-(+)-camphorsulfonate (Formula F6-CSA) product at about 45° C. under vacuum for NLT 12 h. Yield: 23.0 kg, 42 mol, 37.3% with 99.6% purity.

Step D: Synthesis of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (Formula I Intermediate, see FIG. 5 and FIG. 9).

Charge methylene chloride (120 L, 5.50 V) and (2R,3R,11bR)-3-isobutyl-9,10-dimethoxy-2,3,4,6,7,11b-hexahydro-1H-pyrido[2,1-a]isoquinolin-2-ol (S)-(+)-camphorsulfonate (Formula F6-CSA, 21.8 kg, 40 mol, 1.00 equiv) into reactor A. Add 1 N sodium hydroxide (14.8 kg, 111 mol, 2.80 equiv) at about 25° C. Stir for NLT 15 min and split the layers. Wash the organic layer with demineralized water (33 L, 1.50 V). Charge Boc-L-valine (Formula F7,10.2 kg, 47 mol, 1.19 equiv) and 4-dimethylaminopyridine (1.3 kg, 11 mol, 0.27 equiv), cool to about 2° C. and inertise by 4 nitrogen pressure/decompression cycles and sparging with nitrogen. Charge EDC.HCl (13.3 kg, 69 mol, 1.75 equiv) in portions keeping the temperature at about 2° C. Heat to about 25° C. and stir for about 2 h. Add a 0.15N citric acid solution (112.5 kg, 17 mol, 0.42 equiv), stir for NLT 15 min and split the layers. Wash the organic layer with demineralized water (65 L, 3.00 V). Swap the solvent to acetonitrile by 2 put and take distillations at 3.00 V with acetonitrile (109 L, 5.00 V×2). Charge a solution of para-toluenesulfonic acid (15.8 kg, 83 mol, 2.10 equiv) in acetonitrile (76 L, 3.50 V). Heat to about 65° C., stir for about 12 h then cool to about 20° C. and stir for about 2 h. Isolate the solid by filtration. Wash the cake with acetonitrile (65 L, 3.00 V). Dry the wet (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (Formula I) material at about 50° C. under vacuum for NLT 12 h. Yield: 26.2 kg, 34 mol, 85.8% with 99.1% purity.

Step E: Synthesis of (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (Formula I, see FIG. 6 and FIG. 10).

Charge methanol (25 L, 1.00 V), (2R,3R,11bR)-3-isobutyl-9,10-dimethoxy-2,3,4,6,7,11 b-hexahydro-1H-pyrido[2,1-a]isoquinolin-2-yl (S)-2-amino-3-methylbutanoate di(4-methylbenzenesulfonate) (Formula I) material from Step D (24.5 kg, 32 mol, 1.00 equiv) and acetonitrile (49 L, 2.00 V) into reactor A. Heat to about 25° C., polish filter the solution into reactor B. Rinse reactor A, the filter and the transfer line with a mixture of methanol (5 L, 0.20 V) and acetonitrile (10 L, 0.40 V). Charge acetonitrile (39 L, 1.60 V) into reactor B. Distill at about 42° C. Pᵢ=400 to 200 mbar keeping the volume constant at 135 L (5.50 V) while adding acetonitrile (37 L, 1.50 V). Seed with the compound of Formula I (0.02 kg, 0.1 wt %). Distill at about 42° C. Pᵢ=400 to 200 mbar keeping the volume constant at 135 L (5.50 V) while adding acetonitrile (86 L, 3.50 V). Charge acetonitrile (25 L, 1.00 V) at about 42° C., cool to about 20° C. over NLT 4 h and stir for NLT 2 h. Isolate the solid by filtration. Wash the cake with acetonitrile (74 L, 3.00 V). Dry the wet (2R,3R,11bR)-3-isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) (Formula I) compound (DS) at about 50° C. under vacuum for NLT 12 h. Yield: 23.7 kg, 31 mol, 97.1% with 99.5% purity, X-ray powder diffraction (XRPD, see FIG. 1), and differential scanning calorimetry (DSC, see FIG. 2).

Four additional batches (i.e., 084, 085, 086, and 087) were prepared in a similar as described above. The peak temperatures for each are shown below in the Table 4.

TABLE 4

| | Example 1 | Batch | Batch | Batch | Batch |
|---|---|---|---|---|---|
| DSC | Step E | 084 | 085 | 086 | 087 |
| Peak | 243.8° C. | 243.3° C. | 243.7° C. | 243.8° C. | 244.2° C. |
| Temperature | | | | | |

### Example 2: p-Toluenesulfonic Acid Determination

The determination of % area of p-toluenesulfonic acid in the Formula I compound prepared by Step D is performed by HPLC. Separation is based on a gradient, reverse-phase HPLC method with ultraviolet (UV) detection.

Equipment and Equipment Conditions:

The following equipment is needed to perform the HPLC method for the determination of assay in the compound of Formula I.

HPLC system equipped with UV variable wavelength or a photodiode array detector, gradient capabilities, and electronic data collection and processing, or equivalent.
Column: Phenomenex Kinetex XB-C18, 4.6 mm×150 mm, 2.6 m
Column Heater capable of controlling the temperature at 50° C.±2° C.
Autosampler capable of injecting 3 μL
Balance capable of accurately weighing to 0.1 mg
Analytical balance capable of accurately weighing to 0.01 mg
Water purification system, Milli-Q or equivalent
Sonicator
0.45 m membrane filter
pH Meter
Class A volumetric glassware

The parameters and conditions used for the HPLC method are described in Table 5. The gradient conditions are described in Table 6.

TABLE 5
HPLC Chromatographic Conditions

| Parameter | Condition/Setting |
|---|---|
| Column | Phenomenex Kinetex XB-C18, 4.6 mm × |
| | 150 mm, 2.6 μm |
| Column Temperature | 50° C. |
| Detection | UV at 261 nm |
| Flow Rate | 0.8 mL/minute |
| Injection Volume | 3 μL |
| Conc. of Formula I | 4 mg/mL |
| (Ditosylate) | |
| Autosampler Temperature | Ambient |
| Mobile Phase A | 50 mM Ammonium Formate, 0.1% Formic Acid |
| | in Deionized (DI) Water, pH 3.95 |
| Mobile Phase B | Acetonitrile |
| Gradient | See Table 6 for gradient conditions |
| Run Time | 26 minutes (inhibit integration after 20 minutes) |

TABLE 6
HPLC Gradient Conditions

| Time | Mobile | Mobile |
|---|---|---|
| (minutes) | Phase A (%) | Phase B (%) |
| 0 | 90 | 10 |
| 1 | 90 | 10 |
| 9 | 65 | 35 |
| 18.5 | 10 | 90 |
| 19.5 | 10 | 90 |
| 20 | 90 | 10 |
| 26 | 90 | 10 |

Reagents and Reference Standards:

The reagents and reference standards used in this method are listed in Table 7.

TABLE 7
Reagents and Reference Standards used in the HPLC Method

| | |
|---|---|
| Material | Grade ^{a} |

Reagents

| | |
|---|---|
| DI Water | Milli-Q 18 MΩ or equivalent |
| Formic Acid, Ampules, 99% | High Purity-Thermo Scientific or |
| | equivalent |
| Ammonium Formate | HPLC |
| Acetonitrile | HPLC |

Reference Standards (RS)

| | |
|---|---|
| Compound of Formula 1 | RS |

^{a} Reagents of comparable or higher purity may be used

### Preparation of Solutions

Note: Preparations may be scaled as needed.

Mobile Phase A (50 mM Ammonium Formate, 0.1% Formic Acid in DI Water).

Accurately weigh approximately 3.15 grams of ammonium formate into a suitable container and add 500 mL DI water. With a volumetric pipette, add 1.0 mL of formic acid into the container and swirl to mix until all solids are dissolved. Add an additional 500 mL DI water and record the pH of the solution. If the pH of the solution is not between 3.90 and 4.00, re-prepare or adjust the pH with either formic acid or ammonium formate. Filter the solution through a 0.45 m membrane filter and degas.

Diluent (DI Water:Acetonitrile, 50:50, v/v).

Add 500 mL DI water and 500 mL acetonitrile to a suitable container and mix well.

### Preparation of Standard Solutions

Note: Standard solutions are stable for 4 days at ambient laboratory conditions.

Working Standard Solution (4 mg/mL of compound of Formula I).

Accurately weigh approximately 100 mg of the compound of Formula I RS into a 25-mL volumetric flask. Add approximately 20 mL diluent to the flask and mix well by swirling. Sonicate if necessary, to dissolve solids. Dilute to volume with diluent and mix well by inversion.

### Preparation of Sample Solutions

Note: Sample solutions are stable for 4 days at ambient laboratory conditions.

Sample Solution (4 mg/mL of compound of Formula I).

Accurately weigh approximately 100 mg of the compound of Formula I sample into a 25-mL volumetric flask. Add approximately 20 mL diluent to the flask and mix well by swirling.

Sonicate if necessary, to dissolve solids. Dilute to volume with diluent and mix well by inversion.

Procedure:

Equilibrate the HPLC column at the gradient starting conditions (see Table 6) for at least one hour or until a stable baseline is achieved. Perform the sample and standard injections using the sequence in Table 8.

TABLE 8
Injection Sequence of the HPLC Method

| Sample | Number of Injections |
|---|---|
| Blank (Diluent) | 2 or more |
| Working Standard Solution | 5 |
| Sample Solution | 1 |
| Blank (Diluent) | 1 or more |

System Suitability:

Blank—There must be no interfering peaks (>0.05%) at the retention time of the peaks of interest.

Working Standard Solution—The % relative standard deviation (RSD) of the response factor and retention times of the compound of Formula I and PTSA in the first five injections must be not more than (NMT) 1.0%. The tailing factor of the compound of Formula I peak in the first five injections must be NMT 2.0.

Calculations:

PTSA % area (p-Toluene sulfonic acid) in sample solution:

PTSA % area=(A_{PTSA})+(A_{API}+A_{PTSA})×100
where:
A_{PTSA}=Area response for PTSA (area counts)
A_{API}=Area response for the compound of Formula I (area counts)

Reporting of Results:

PTSA % area

PTSA % area from 40.35% to 41.21% area indicates that PTSA/Formula I Stoichiometry is 2.0.

PTSA % area >41.21% area indicates that excess PTSA exists. A PTSA % area of 42.35% area indicates that a 0.1 equivalent excess PTSA exists.

PTSA % area <40.35% area indicates that insufficient PTSA reacts with the compound of Formula F8 and will result in low yield.

PTSA determination results are summarized in Table 9, where the PTSA/DS ratio refers to the PTSA/Formula I stoichiometry.

TABLE 9
Summary of PTSA determination by HPLC

| | PTSA/ | PTSA % |
|---|---|---|
| Sample # | DS ratio | area vs DS |
| 1 (500 g scale) | 2 | 41.03 |
| 2 (spike 0.1 eq | 2.1 | 42.35 |
| PTSA) | | |
| 3 (RS) | 2 | 41.06 |
| 4 (before | 2 | 41.11 |
| recrystallization) | | |
| 5 | 2 | 41.21 |
| 6 | 2 | 41.07 |
| 7 | 2 | 41.21 |
| 8 | 2 | 40.35 |
| 9 | 2 | 40.79 |
| 10 | 2 | 40.96 |
| 11 | 2 | 41.01 |

### Example 3: Analytical Characterization of (2R,3R,11bR)-3-Isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate), Compound of Formula I

Four separate batches of (2R,3R,11bR)-3-Isobutyl-9,10-dimethoxy-2,3,4,6,7,11b-hexahydro-1H-pyrido[2,1-a]isoquinolin-2-yl (S)-2-amino-3-methylbutanoate di(4-methylbenzenesulfonate) were manufactured in a similar manner as described herein (i.e., Example 1) in compliance with applicable cGMPs for GLP use. Certain data are shown below in Table 10 for Example 1, Step E (i.e., Batch 073) and the four batches (i.e., Batches 084, 085, 086, and 087).

TABLE 10

| Test | Batch 073 | Batch 084 | Batch 085 | Batch 086 | Batch 087 |
|---|---|---|---|---|---|
| HPLC Assay (Purity) | 100 % | 100 % | 100 % | 100 % | 100 % |
| Compound of Formula 1 | | | | | |
| Compound 2A | <0.05 % | <0.05 % | <0.05 % | < 0.05 % | <0.05 % |
| Compound 2B | <0.05 % | <0.05 % | <0.05 % | <0.05 % | <0.05 % |
| Compound 2C | <0.05 % | <0.05 % | <0.05 % | <0.05 % | <0.05 % |
| Compound 2D | <0.05 % | <0.05 % | <0.05 % | <0.05 % | <0.05 % |
| Compound 2E | <0.05 % | <0.2 % | <0.2 % | <0.2 % | <0.2 % |
| (R,R,R,R-enantiomcr) | | | | | |
| Acetonitrile | 83 ppm | <10 ppm | 15 ppm | 35 ppm | 24 ppm |
| (residual solvent) | | | | | |
| Ethanol | <100 ppm | <100 ppm | <100 ppm | <100 ppm | <100 ppm |
| (residual solvent) | | | | | |
| Dichloromethane | <30 ppm | <30 ppm | <30 ppm | <30 ppm | <30 ppm |
| (residual solvent) | | | | | |
| Methanol | <60 ppm | <60 ppm | <60 ppm | < 60 ppm | <60 ppm |
| (residual solvent) | | | | | |
| Particle size distribution, | 3 μm | 3 μm | 3 μm | 3 μm | 3 μm |
| D10 | | | | | |
| Particle size distribution, | 14 μm | 13 μm | 13 μm | 14 μm | 13 μm |
| D50 | | | | | |
| Particle size distribution, | 45 μm | 43 μm | 44 μm | 48 μm | 45 μm |
| D90 | | | | | |
| Water (KF) | 0.4 % | 0.5 % | 0.6 % | 0.5 % | 0.6 % |
| DSC (Peak temperature) | 243.8° C. | 243.3° C. | 243.7° C. | 243.8° C. | 244.2° C. |
| XRPD | Form I | Form I | Form I | Form I | Form I |

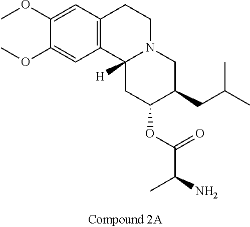 [SMILES: COc1cc2c(cc1OC)[C@H]1C[C@@H](OC(=O)[C@H](C)N)[C@H](CC(C)C)CN1CC2]

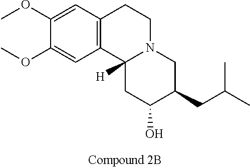 [SMILES: COc1cc2c(cc1OC)[C@H]1C[C@@H](O)[C@H](CC(C)C)CN1CC2]

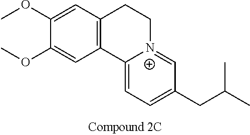 [SMILES: COc1cc2c(cc1OC)-c1ccc(CC(C)C)c[n+]1CC2]

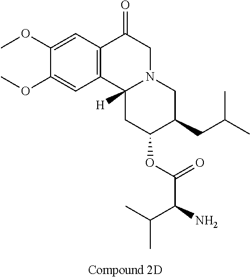 [SMILES: COc1cc2c(cc1OC)[C@H]1C[C@@H](OC(=O)[C@@H](N)C(C)C)[C@H](CC(C)C)CN1CC2=O]

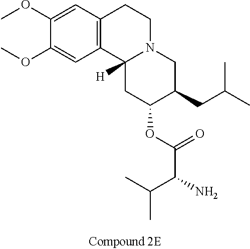 [SMILES: COc1cc2c(cc1OC)[C@H]1C[C@@H](OC(=O)[C@H](N)C(C)C)[C@H](CC(C)C)CN1CC2]

A representative list of for specification for each of the tests used to analyze the four batches are provided below in Table 11.

TABLE 11

| Test | Specifications | Test | Specifications |
|---|---|---|---|
| HPLC Assay | Min 99.5% | Dichloromethane | Max. 600 |
| (Purity) | | (residual solvent) | ppm |
| Compound 2A | Max. 0.3% | Methanol | Max. 3000 |
| | | (residual solvent) | ppm |
| Compound 2B | Max. 0.3% | Particle size | 1-8 μm |
| | | distribution, D10 | |
| Compound 2C | Max. 0.2% | Particle size | 4-27 μm |
| | | distribution, D50 | |
| Compound 2D | Max. 0.3% | Particle size | 19-62 μm |
| | | distribution, D90 | |
| Compound 2E | Max. 0.5% | Water (KF) | Max. 2% |
| (R,R,R,R-enantiomer) | | | |
| Acetonitrile | Max. 410 ppm | DSC (Peak | 237-247° C. |
| (residual solvent) | | temperature) | |
| Ethanol | Max. 5000 ppm | XRPD | Form I |
| (residual solvent) | | | |

Various modifications of the disclosure, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in their entirety.

What is claimed is:

1. A process of preparing a compound of Formula I: 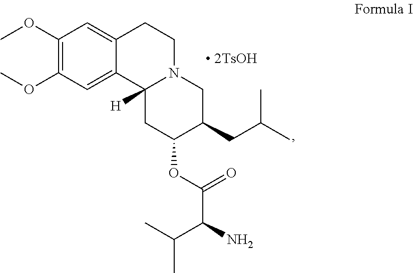 [SMILES: COc1cc2c(cc1OC)[C@H]1C[C@@H](OC(=O)[C@@H](N)C(C)C)[C@H](CC(C)C)CN1CC2]
comprising reacting a compound of Formula F8: 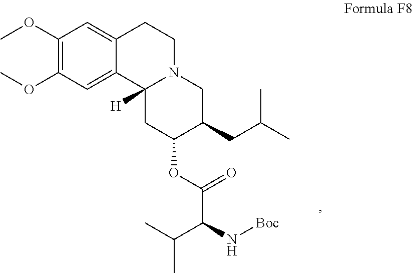 [SMILES: COc1cc2c(cc1OC)[C@H]1C[C@@H](OC(=O)[C@@H](NC(=O)OC(C)(C)C)C(C)C)[C@H](CC(C)C)CN1CC2]
with p-toluenesulfonic acid in a solvent comprising acetonitrile or isopropyl acetate, to afford the compound of Formula I.

2. The process of claim 1, wherein the ratio of p-toluenesulfonic acid to the compound of Formula F8 is about 2.0:1 to about 2.2:1 molar equivalents.

3. The process of claim 1, wherein the ratio of p-toluenesulfonic acid to the compound of Formula F8 is about 2.1:1 molar equivalents.

4. The process of any one of claims 1 to 3, wherein the reacting of the compound of Formula F8 with p-toluenesulfonic acid is carried out at a temperature of about 50° C. to about 75° C.

5. The process of any one of claims 1 to 3, wherein the reacting of the compound of Formula F8 with p-toluenesulfonic acid is carried out at a temperature of about 64° C. to about 66° C.

6. The process of any one of claims 1 to 5, wherein the reacting of the compound of Formula F8 with p-toluenesulfonic acid is carried out over a period sufficient to reduce the presence of the compound of Formula F8 to at least 3% as determined by HPLC.

7. The process of any one of claims 1 to 5, wherein the reacting of the compound of Formula F8 with p-toluenesulfonic acid is carried out over a period of about 10 hours to about 14 hours.

8. The process of any one of claims 1 to 7, wherein after the reacting of the compound of Formula F8 with p-toluenesulfonic acid the process further comprises cooling to a temperature of about 18° C. to about 22° C.

9. The process of claim 8, wherein the temperature is maintained for about 1.8 hours to about 2.2 hours.

10. The process of claim 9, wherein the temperature is maintained with stirring.

11. The process of any one of claims 1 to 10, wherein the compound of Formula I is isolated by washing with acetonitrile and drying at elevated temperature under vacuum.

12. The process of any one of claims 1 to 11, wherein the compound of Formula F8 is prepared by a process comprising reacting a compound of Formula F6: 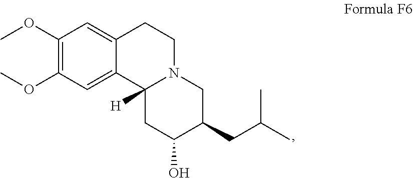 [SMILES: COc1cc2c(cc1OC)[C@H]1C[C@@H](O)[C@H](CC(C)C)CN1CC2]
with a carboxylic acid of Formula F7: 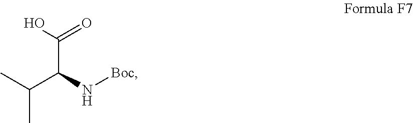
in a solvent, to afford the compound of Formula F8.

13. The process of claim 12, wherein the reacting of the compound of Formula F6 with a carboxylic acid of Formula F7 is performed in a solvent comprising a halogenated hydrocarbon solvent.

14. The process of claim 12, wherein said halogenated hydrocarbon solvent is dichloromethane.

15. The process of any one of claims 12 to 14, wherein the reacting of the compound of Formula F6 with the carboxylic acid of Formula F7 is carried out in the presence of a coupling reagent and a catalytic base.

16. The process of claim 15, wherein the coupling reagent present in the reacting of the compound of Formula F6 with the carboxylic acid of Formula F7 is EDC-HCl.

17. The process of claim 15 or 16, wherein the catalytic base present in the reacting of the compound of Formula F6 with the carboxylic acid of Formula F7 is DMAP.

18. The process of any one of claims 12 to 17, wherein the compound of Formula F6 is prepared by a process comprising reacting a compound of Formula F6-CSA: 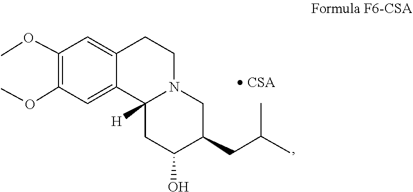 [SMILES: COc1cc2c(cc1OC)[C@H]1C[C@@H](O)[C@H](CC(C)C)CN1CC2]
with a base, to afford the compound of Formula F6.

19. The process of claim 18, wherein the base which is reacted with the compound of Formula F6-CSA is sodium hydroxide.

20. The process of claim 18 or 19, wherein the reacting of the compound of Formula 6-CSA with a base is performed in a solvent comprising water and a halogenated hydrocarbon solvent.

21. The process of claim 20, wherein said halogenated hydrocarbon solvent is dichloromethane.

22. The process of any one of claims 18 to 21, the reacting of the compound of Formula 6-CSA with a base is performed at a temperature of about 22° C. to about 28° C.

23. The process of any one of claims 18 to 22, wherein the compound of Formula F6-CSA is prepared by the process comprising reacting a compound of Formula F5: 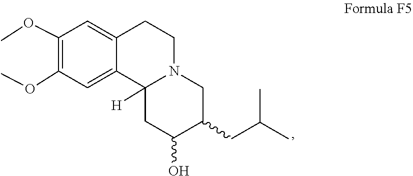 [SMILES: COc1cc2c(cc1OC)C1CC(O)C(CC(C)C)CN1CC2]
with (S)-(+)-camphorsulfonic acid (CSA), to afford the compound of Formula F6-CSA.

24. The process of claim 23, wherein the molar ratio of CSA to the compound of Formula F5 is about 0.7:1 to about 0.9:1.

25. The process of claim 23, wherein the molar ratio of CSA to the compound of Formula F5 is about 0.80:1 to about 0.85:1.

26. The process of any one of claims 23 to 25, wherein said reacting of the compound of Formula F5 is performed in a solvent comprising ethanol and water.

27. The process of claim 26, wherein the solvent is a mixture comprising water and ethanol in a volume ratio of water to ethanol of about 1:14 to about 1:18.

28. The process of claim 26, wherein the solvent is a mixture comprising water and ethanol in a volume ratio of water to ethanol of about 1:15.5 to about 1:16.5.

29. The process of any one of claims 23 to 28, wherein the compound of Formula F6-CSA has an optical purity greater than 99%.

30. The process of any one of claims 23 to 29 wherein the compound of Formula F5 is prepared by the process comprising reacting a compound of Formula F4: 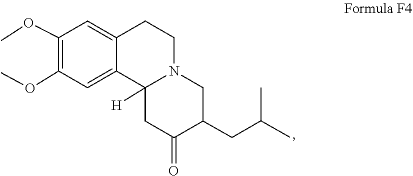 [SMILES: COc1cc2c(cc1OC)C1CC(=O)C(CC(C)C)CN1CC2]
with a reducing agent in a solvent comprising methyl tert-butyl ether (MTBE) and methanol, to afford the compound of Formula F5.

31. The process of claim 30, wherein the solvent comprising methyl tert-butyl ether (MTBE) and methanol further comprises an acid.

32. The process of claim 31, wherein the acid comprises acetic acid.

33. The process of claim 32, wherein the acetic acid is present in about 1.0 to about 1.2 equivalents to the compound of Formula F4.

34. The process of any of claims 30 to 33, wherein the reducing agent is sodium borohydride.

35. The process of any of claims 30 to 34, wherein the reacting of the compound of Formula F4 with a reducing agent is conducted at a temperature of about 20° C. to about 27° C.

36. The process of any one of claims 30 to 35, wherein the compound of Formula F4 is prepared by the process comprising reacting a compound of Formula F3: 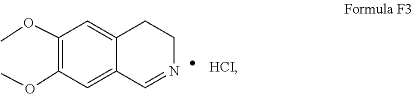
with a compound of Formula F2: 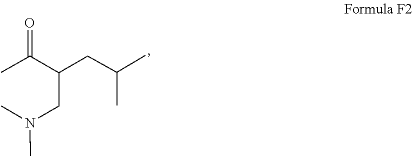
in a solvent comprising isopropanol (IPA) and water, to afford the compound of Formula F4.

37. The process of claim 36, wherein the volume ratio of IPA and water is about 2.2:1 to about 2.4:1.

38. The process of claim 36 or 37, wherein the reacting of the compound of Formula F2 and the compound of Formula F3 takes place in the presence of sodium iodide.

39. The process of claim 38, wherein the molar ratio of sodium iodide to the compound of Formula F3 is about 0.3:1 to 0.5:1.

40. The process of any one of claims 36 to 39, the reacting of the compound of Formula F3 with the compound of Formula F2 is carried out at a temperature of about 36° C. to about 48° C.

41. The process of any one of claims 36 to 40 wherein the compound of Formula F2 is prepared by the process comprising reacting a compound of Formula F1: 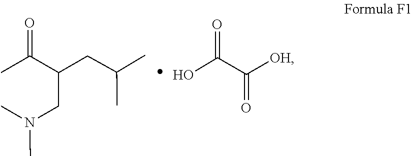
with a base, to afford the compound of Formula F2.

42. The process of claim 41, wherein the base which is reacted with the compound of Formula F1 is potassium hydroxide.

43. The process of claim 41 or 42, wherein said reacting of the compound of Formula F1 is performed in a solvent comprising water and an organic solvent.

44. The process of claim 43, wherein said organic solvent used in the reacting of the compound of Formula F1 is MTBE.

45. The process of any one of claims 41 to 44, wherein the solvent used in the reacting of the compound of Formula F1 is removed after completion of the reaction and replaced with isopropanol.

46. The process of any one of claims 1 to 45, further comprising the step of formulating the compound of Formula I to form a pharmaceutical composition.

47. The process of any one of claims 1 or 45, further comprising the step of formulating the compound of Formula I to form a pharmaceutical composition comprising: silicified microcrystalline cellulose; isomalt; hydroxypropyl methylcellulose; partially pregelatinized maize starch; and magnesium stearate.

48. A process of preparing a crystalline compound of Formula I, 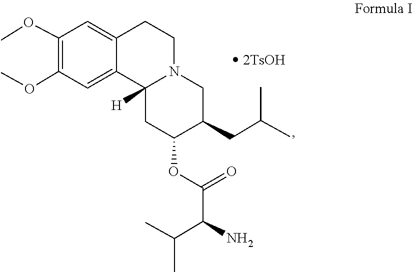 [SMILES: COc1cc2c(cc1OC)[C@H]1C[C@@H](OC(=O)[C@@H](N)C(C)C)[C@H](CC(C)C)CN1CC2]
comprising:
a) dissolving the compound of Formula I in a solvent mixture comprising an alcohol and acetonitrile; and
b) crystallizing the compound of Formula I to afford a crystalline form of the compound of Formula I.

49. The process of claim 48, wherein the volume ratio of alcohol to acetonitrile in the solvent mixture is about 1:1.8 to about 1:2.2.

50. The process of claim 48 or 49, wherein the crystallizing in step b) comprises seeding the resulting solvent and compound mixture with a crystal of the compound of Formula I to form a seed mixture.

51. The process of claim 50, wherein the seed mixture is heated to a temperature of about 39° C. to about 45° C. prior to and/or during seeding.

52. The process of claim 51, wherein after heating the seed mixture, the resulting seed mixture is cooled to a temperature of about 17° C. to about 23° C.

53. The process of any one of claims 48 to 52, wherein the alcohol is methanol.

54. The process of any one of claims 48 to 52, wherein the crystalline form of the compound of Formula I is isolated and drying at elevated temperature under vacuum.

55. The process of any one of claims 48 to 54, wherein the crystalline form of the compound of Formula I is Form I.

56. The process of any one of claims 48 to 55, further comprising the step of formulating the crystalline form of the compound of Formula I to form a pharmaceutical composition.

57. The process of any one of claims 48 to 55, further comprising the step of formulating the compound of Formula I to form a pharmaceutical composition comprising: silicified microcrystalline cellulose; isomalt; hydroxypropyl methylcellulose; partially pregelatinized maize starch; and magnesium stearate.

58. A process of preparing a compound of Formula I: 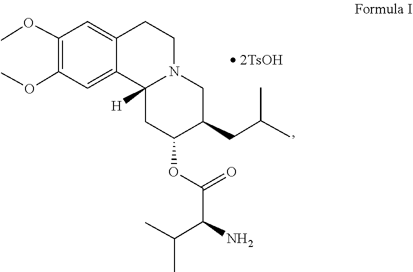 [SMILES: COc1cc2c(cc1OC)[C@H]1C[C@@H](OC(=O)[C@@H](N)C(C)C)[C@H](CC(C)C)CN1CC2]
comprising:
a) reacting a compound of Formula F6-CSA: 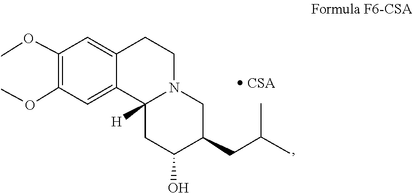 [SMILES: COc1cc2c(cc1OC)[C@H]1C[C@@H](O)[C@H](CC(C)C)CN1CC2]
with a base to afford a compound of Formula F6: 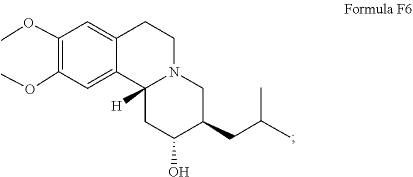 [SMILES: COc1cc2c(cc1OC)[C@H]1C[C@@H](O)[C@H](CC(C)C)CN1CC2]
b) reacting the compound of Formula F6 with a carboxylic acid of Formula F7: 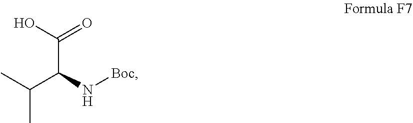
in a solvent to form a compound of Formula F8: 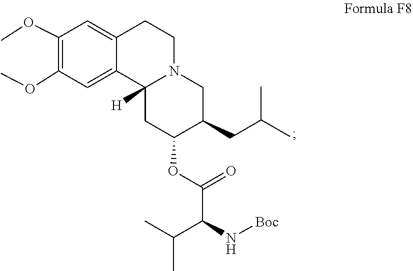 [SMILES: COc1cc2c(cc1OC)[C@H]1C[C@@H](OC(=O)[C@@H](NC(=O)OC(C)(C)C)C(C)C)[C@H](CC(C)C)CN1CC2]
c) reacting the compound of Formula F8 with p-toluenesulfonic acid in a solvent comprising acetonitrile or isopropyl acetate, to afford the compound of Formula I.

59. The process of claim 58, wherein the ratio of p-toluenesulfonic acid to the compound of Formula F8 is about 2.0:1 to about 2.2:1 molar equivalents.

60. The process of claim 58, wherein the ratio of p-toluenesulfonic acid to the compound of Formula F8 is about 2.1:1 molar equivalents.

61. The process of any one of claims 58 to 60, wherein the reacting of the compound of Formula F8 with p-toluenesulfonic acid is carried out at a temperature of about 50° C. to about 75° C.

62. The process of any one of claims 58 to 60, wherein the reacting of the compound of Formula F8 with p-toluenesulfonic acid is carried out at a temperature of about 64° C. to about 66° C.

63. The process of any one of claims 58 to 62 wherein the reacting of the compound of Formula F8 with p-toluenesulfonic acid is carried out over a period sufficient to reduce the presence of the compound of Formula F8 to at least 3% as determined by HPLC.

64. The process of any one of claims 58 to 63, wherein the reacting of the compound of Formula F8 with p-toluenesulfonic acid is carried out over a period of about 10 hours to about 14 hours.

65. The process of any one of claims 58 to 64, wherein after the reacting of the compound of Formula F8 with p-toluenesulfonic acid the process further comprises cooling to a temperature of about 18° C. to about 22° C.

66. The process of claim 65, wherein the temperature is maintained for about 1.8 hours to about 2.2 hours.

67. The process of claim 66, wherein the temperature is maintained with stirring.

68. The process of any one of claims 58 to 67, wherein the compound of Formula I is isolated by washing with acetonitrile and drying at elevated temperature under vacuum.

69. The process of any one of claims 58 to 68, wherein the reacting of the compound of Formula F6 with a carboxylic acid of Formula F7 is performed in a solvent comprising a halogenated hydrocarbon solvent.

70. The process of any one of claims 58 to 68, wherein said halogenated hydrocarbon solvent is dichloromethane.

71. The process of any one of claims 58 to 70, wherein the reacting of the compound of Formula F6 with the carboxylic acid of Formula F7 is carried out in the presence of a coupling reagent and a catalytic base.

72. The process of claim 71, wherein the coupling reagent present in the reacting of the compound of Formula F6 with the carboxylic acid of Formula F7 is EDC-HCl.

73. The process of claim 71 or 72, wherein the catalytic base present in the reacting of the compound of Formula F6 with the carboxylic acid of Formula F7 is DMAP.

74. The process of any one of claims 58 to 73, wherein the base which is reacted with the compound of Formula F6-CSA is sodium hydroxide.

75. The process of claim 74, wherein the reacting of the compound of Formula 6-CSA with a base is performed in a solvent comprising water and a halogenated hydrocarbon solvent.

76. The process of claim 75, wherein said halogenated hydrocarbon solvent is dichloromethane.

77. The process of any one of claims 58 to 76, the reacting of the compound of Formula 6-CSA with a base is performed at a temperature of about 22° C. to about 28° C.

78. A process of preparing a compound of Formula F6-CSA: 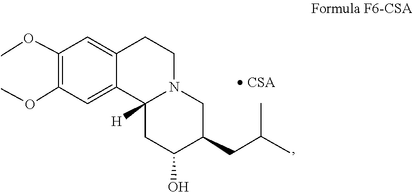 [SMILES: COc1cc2c(cc1OC)[C@H]1C[C@@H](O)[C@H](CC(C)C)CN1CC2]
comprising reacting a compound of Formula F5: 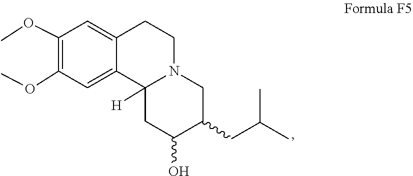 [SMILES: COc1cc2c(cc1OC)C1CC(O)C(CC(C)C)CN1CC2]
with (S)-(+)-camphorsulfonic acid (CSA), wherein the molar ratio of CSA to the compound of Formula F5 is between 0.7:1 and 0.9:1, to afford the compound of Formula F6-CSA.

79. The process of claim 78, wherein the molar ratio of CSA to the compound of Formula F5 is about 0.7:1 to about 0.9:1.

80. The process of claim 78, wherein the molar ratio of CSA to the compound of Formula F5 is about 0.80:1 to about 0.85:1.

81. The process of any one of claims 78 to 80, wherein said reacting of the compound of Formula F5 is performed in a solvent comprising ethanol and water.

82. The process of claim 81, wherein the solvent is a mixture comprising water and ethanol in a volume ratio of water to ethanol of about 1:14 to about 1:18.

83. The process of claim 82, wherein the solvent is a mixture comprising water and ethanol in a volume ratio of water to ethanol of about 1:15.5 to about 1:16.5.

84. The process of any one of claims 78 to 83, wherein the compound of Formula F6-CSA has an optical purity greater than 99%.

85. A process of preparing a compound of Formula F5: 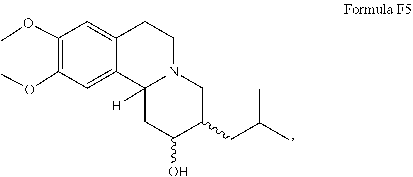 [SMILES: COc1cc2c(cc1OC)C1CC(O)C(CC(C)C)CN1CC2]
comprising reacting a compound of Formula F4: 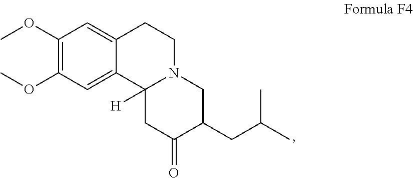 [SMILES: COc1cc2c(cc1OC)C1CC(=O)C(CC(C)C)CN1CC2]
with a reducing agent in a solvent comprising methyl tert-butyl ether (MTBE) and methanol, to afford the compound of Formula F5.

86. The process of claim 85, wherein the solvent comprising methyl tert-butyl ether (MTBE) and methanol further comprises an acid.

87. The process of claim 86, wherein the acid comprises acetic acid.

88. The process of claim 87, wherein the acetic acid is present in about 1.0 to about 1.2 equivalents to the compound of Formula F4.

89. The process of any of claims 85 to 88, wherein the reducing agent is sodium borohydride.

90. The process of any of claims 85 to 89, wherein the reacting of the compound of Formula F4 with a reducing agent is conducted at a temperature of about 20° C. to about 27° C.

91. A process of preparing a compound of Formula F4: 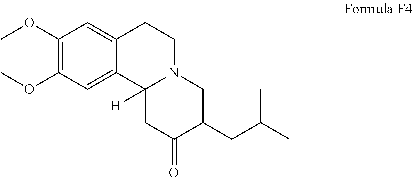 [SMILES: COc1cc2c(cc1OC)C1CC(=O)C(CC(C)C)CN1CC2]
comprising:
a) reacting a compound of Formula F1: 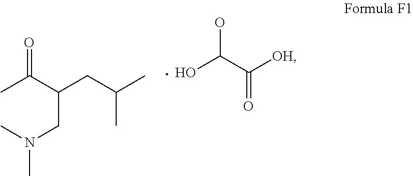
with a base to afford a compound of Formula F2: 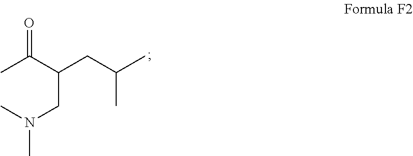
b) reacting the compound of Formula F2 with a compound of Formula F3: 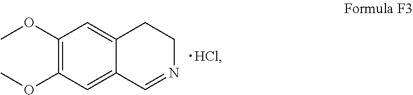
in the presence of sodium iodide in a solvent comprising isopropanol (IPA) and water, to afford the compound of Formula F4.

92. The process of claim 91, wherein the volume ratio of IPA and water is about 2.2:1 to about 2.4:1.

93. The process of claim 91 or 92, wherein the molar ratio of sodium iodide to the compound of Formula F3 is about 0.3:1 to 0.5:1.

94. The process of any one of claims 91 to 93, wherein the reacting of the compound of Formula F3 with the compound of Formula F2 is carried out at a temperature of about 36° C. to about 48° C.

95. The process of any one of claims 91 to 94, wherein the base which is reacted with the compound of Formula F1 is potassium hydroxide.

96. The process of any one of claims 91 to 95, wherein the reacting of the compound of Formula F1 is performed in a solvent comprising water and an organic solvent.

97. The process of claim 96, wherein the organic solvent used in the reacting of the compound of Formula F1 is MTBE.

98. The process of claim 96 or 97, wherein the solvent used in the reacting of the compound of Formula F1 is removed after completion of the reaction and replaced with isopropanol.

99. A process of preparing a compound of Formula I: 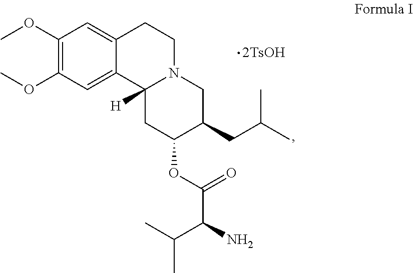 [SMILES: COc1cc2c(cc1OC)[C@H]1C[C@@H](OC(=O)[C@@H](N)C(C)C)[C@H](CC(C)C)CN1CC2]
comprising:
a) reacting a compound of Formula F1: 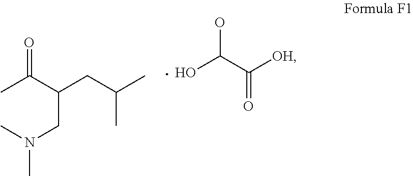
with a base, to afford a compound of Formula F2: 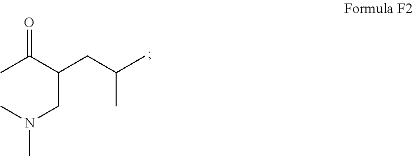
b) reacting a compound of Formula F3: 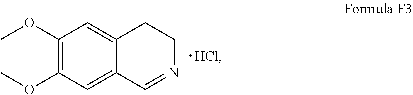
with the compound of Formula F2 in a solvent comprising isopropanol (IPA) and water to afford a compound of Formula F4: 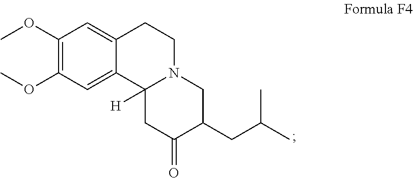 [SMILES: COc1cc2c(cc1OC)C1CC(=O)C(CC(C)C)CN1CC2]
c) reacting the compound of Formula F4 with a reducing agent in a solvent comprising methyl tert-butyl ether (MTBE) and methanol to afford a compound of Formula F5: 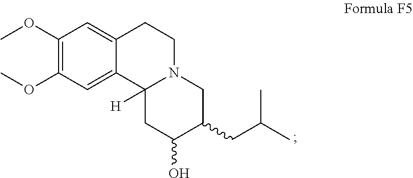 [SMILES: COc1cc2c(cc1OC)C1CC(O)C(CC(C)C)CN1CC2]
d) reacting the compound of Formula F5 with (S)-(+)-camphorsulfonic acid (CSA) to afford a compound of Formula F6-CSA: 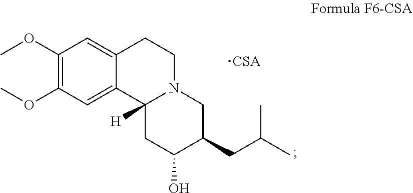
e) reacting the compound of Formula F6-CSA with a base to afford a compound of Formula F6: 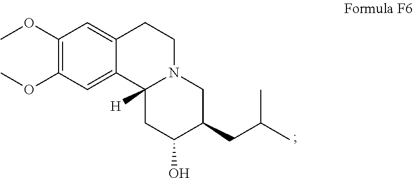 [SMILES: COc1cc2c(cc1OC)[C@H]1C[C@@H](O)[C@H](CC(C)C)CN1CC2]
f) reacting the compound of Formula F6 with a carboxylic acid of Formula F7: 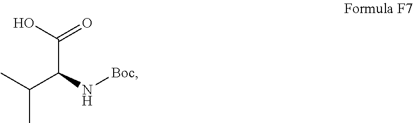
in a solvent to afford a product of Formula F8: 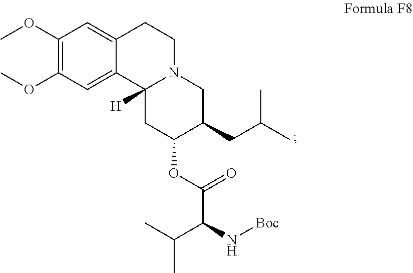 [SMILES: COc1cc2c(cc1OC)[C@H]1C[C@@H](OC(=O)[C@@H](NC(=O)OC(C)(C)C)C(C)C)[C@H](CC(C)C)CN1CC2]
g) reacting the product of Formula F8 with p-toluenesulfonic acid in acetonitrile or isopropyl acetate, to afford a material comprising the compound of Formula I; and
h) crystallizing the material comprising the compound of Formula I, comprising:
i) dissolving the material comprising the compound of Formula I in a solvent mixture comprising methanol and acetonitrile; and
ii) crystallizing the compound of Formula I from the solvent mixture to afford the compound of Formula I.

100. The process of claim 99, wherein the compound of Formula I is crystalline Form I.

101. A process for preparing a pharmaceutical composition comprising: preparing a compound of Formula I according to any one of claims 1 to 45, 48 to 55, 58 to 77, 99, and 100, and formulating the compound of Formula I with a pharmaceutically acceptable carrier and/or diluent.

102. The process of claim 101, wherein the compound of Formula I is prepared by the process of claim 1, 48, 58, or 100.

103. The process of claim 101 or 102, wherein the pharmaceutically acceptable carrier and/or diluent comprises silicified microcrystalline cellulose; isomalt; hydroxypropyl methylcellulose; partially pregelatinized maize starch; and magnesium stearate.
